(19) United States
(12) Patent Application Publication
(10) Pub. No.: US 2025/0302823 A1
(43) Pub. Date: Oct. 2, 2025

(54) COMPOSITIONS AND METHODS FOR TREATING HEPATIC PORPHYRIAS WITH GLYCINE TRANSPORT INHIBITORS

(71) Applicant: Disc Medicine, Inc., Watertown, MA (US)

(72) Inventors: Maria Gabriela Beconi, Watertown, MA (US); Min Wu, Watertown, MA (US); Vu Hong, Watertown, MA (US)

(21) Appl. No.: 18/866,586

(22) PCT Filed: May 30, 2023
(86) PCT No.: PCT/US2023/023886

§ 371 Date: Nov. 18, 2024

Related U.S. Application Data
(60) Provisional application No. 63/347,415, filed on May 31, 2022.

(51) Int. Cl. A61K 31/496; A61P 1/16; A61P 3/00; A61P 7/00

(52) U.S. Cl. CPC A61K 31/496; A61P 1/16; A61P 3/00; A61P 7/00

(57) ABSTRACT

The present embodiments are directed to methods of using glycine transporter inhibitors, such as GlyT1 inhibitors, or pharmaceutically acceptable salts, solvates or prodrugs thereof, or pharmaceutical compositions thereof, for preventing or treating a hepatic porphyria, and related syndromes thereof.

### RELATED APPLICATIONS

This application claims the benefit of priority to U.S. Provisional Patent Application No. 63/347,415, filed May 31, 2022. The specification of the foregoing application is incorporated herein by reference in its entirety.

### FIELD

Embodiments disclosed herein are directed to methods and uses to prevent or treat a hepatic porphyria with glycine transporter inhibitors, such as, but not limited to, GlyT1 inhibitors, or pharmaceutically acceptable salts, solvates, prodrugs thereof, or pharmaceutical compositions thereof.

### BACKGROUND

Porphyrias are a family of disorders resulting from the deficient activity of specific enzymes in the heme biosynthetic pathway, also referred to herein as the porphyrin pathway. Each porphyria is classified as hepatic or erythropoietic based upon the organ system in which the heme precursor is overproduced. They are also classified as acute or non-acute based on their clinical presentation. Deficiency in the enzymes of the porphyrin pathway leads to insufficient heme production and to an accumulation of porphyrin precursors (e.g., ALA and PBG) and porphyrins, which are toxic to tissue in high concentrations.

Acute hepatic porphyrias include acute intermittent porphyria (AIP), variegate porphyria (VP), hereditary coproporphyria (HCP), and aminolevulinic acid dehydratase deficient porphyria (ADP), and often lead to serious abdominal, psychiatric, neurologic, or cardiovascular symptoms. Each acute hepatic porphyria results from a genetic defect leading to deficiency in one of the enzymes of the heme synthesis pathway in the liver. Porphyria cutanea tarda (PCT) is a non-acute hepatic porphyria in which patients often present with blisters, bullae, milia, and hypertrichosis on cheeks, temples, and eyebrows. In addition, there is a rare homozygous recessive form of PCT known as hepatoerythropoietic porphyria (HEP).

In the acute porphyrias (e.g., AIP, VP, HCP and ADP) the respective enzyme deficiencies result in hepatic production and accumulation of one or more substances (e.g., porphyrins and/or porphyrin precursors such as ALA and/or PBG) that can be neurotoxic and can result in the occurrence of acute attacks. If not treated properly, quadriplegia, respiratory impairment, and death may ensue. These genetic disorders are rare and often difficult to diagnose. Approximately 1 in 10,000 Europeans have a mutation in one of the genes that cause AIP, VP, or HCP. However, the majority (80-90%) of confirmed genetic carriers remain asymptomatic, and others experience one or a few acute attacks throughout life.

The current therapy for acute neurological attacks includes the intravenous administration of hemin (Panhematin®, Lundbeck or Normosang®, Orphan Europe), which provides exogenous heme for the negative feedback inhibition of ALAS1, and thereby, decreases production of ALA and PBG. Hemin is used for the treatment during an acute attack and for prevention of attacks, particularly in women having an acute porphyria who experience frequent attacks due to hormonal changes during their menstrual cycles. While patients generally respond well, its effect is slow, typically taking two to four days or longer for urinary ALA and PBG concentrations to trend towards normal levels. As the intravenous hemin is rapidly metabolized, three to four infusions are usually necessary to effectively treat or prevent an acute attack. In addition, repeated infusions may cause iron overload and phlebitis, which may compromise peripheral venous access.

Givosiran (Givlaari®), an aminolevulinate synthase 1-directed small interfering ribonucleic acid (siRNA) is also used to treat patients with acute hepatic porphyrias by targeting and degrading ALAS1 mRNA in hepatocytes using RNA interference. The concerned risks associated with the use of givosiran include anaphylactic reactions, liver toxicity, and renal toxicity. For example, 15% patients in givosiran clinical trials showed transaminase (ALT) elevations 3 times the upper limit of normal. Additionally, 15% of patients receiving givosiran have renal-related adverse reactions including elevated serum creatinine levels and decreased estimated glomerular filtration rate. One final treatment is orthotrophic liver transplantation. While orthotrophic liver transplantation is curative, this procedure has significant morbidity and mortality and the availability of liver donors is limited. Accordingly, there is a need for new methods and compositions for treating and/or preventing hepatic porphyrias. The methods and use of glycine transporter inhibitors, such as, but not limited to, GlyT1 inhibitors, described herein fulfill these needs as well as others.

### SUMMARY OF THE APPLICATION

In certain aspects, the disclosure provides for a method of treating a hepatic porphyria in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt.

In certain aspects, the disclosure provides for a method of preventing, treating, or reducing the progression rate and/or severity of a hepatic porphyria in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt.

In certain aspects, the disclosure provides for a method of preventing, treating, or reducing the progression rate and/or severity of one or more complications of a hepatic porphyria in a subject, the method comprising administering to the subject a pharmaceutical composition comprising one or more GlyT1 inhibitor, or a pharmaceutically acceptable salt thereof, or a prodrug of the one or more GlyT1 inhibitor or its pharmaceutically acceptable salt. In some embodiments, the one or more complications of hepatic porphyria is selected from the group consisting of: acute photosensitivity, cutaneous photosensitivity, severe abdominal pain, neuropsychiatric symptoms, autonomic neuropathy, peripheral motor neuropathy, electrolyte disturbances, nausea, vomiting, constipation, diarrhea, difficulty urinating, ileus, paresthesia, insomnia, restlessness, agitation, anxiety, confusion, hallucinations, psychosis, convulsions, pain associated with neuropathy, muscle paralysis, tetraparesis, decreased breathing, respiratory arrest, hyponatremia, tachycardia, hypertension, increased heart rate, increased blood pressure, red urine, dark urine, hepatocellular carcinoma, hypertensive renal damage, chronic kidney disease, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, and terminal phase liver disease. In some embodiments, the hepatic porphyria is an acute hepatic porphyria. In some embodiments, the acute hepatic porphyria is acute intermittent porphyria (AIP). In some embodiments, the acute hepatic porphyria is ALA dehydratase porphyria (ADP). In some embodiments, the acute hepatic porphyria is variegate porphyria (VP). In some embodiments, the acute hepatic porphyria is hereditary coproporphyria (HCP). In some embodiments, the acute hepatic porphyria is harderoporphyria. In some embodiments, the hepatic porphyria is non-acute hepatic porphyria. In some embodiments, the non-acute hepatic porphyria is familial and sporadic porphyria cutanea tarda (PCT). In some embodiments, the non-acute hepatic porphyria is hepatoerythropoietic porphyria (HEP). In some embodiments, the acute photosensitivity is due to sun exposure. In some embodiments, the method increases pain free light exposure in the subject. In some embodiments, the method decreases light sensitivity in the subject.

In certain aspects, the disclosure provides for a method of inhibiting 5-aminolevulinic acid (5-ALA) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has a hepatic porphyria.

In certain aspects, the disclosure provides for a method of inhibiting coproporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting zinc-protoporphyrin IX (ZPPIX) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has ALA dehydratase porphyria (ADP).

In certain aspects, the disclosure provides for a method of inhibiting porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting 5-aminolevulinic acid (5-ALA) and porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting hydroxymethylbilane (HMB) synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting uroporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting heptacarboxyl-porphyrin synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting isocoproporphyrin synthesis in vivo, comprising administering to a subject a GlyT1 inhibitor, or a pharmaceutically acceptable salt thereof, or a prodrug of the GlyT1 inhibitor or its pharmaceutically acceptable salt.

In certain aspects, the disclosure provides for a method of inhibiting synthesis of a porphyrin or porphyrin precursor in vivo, comprising administering to a subject a GlyT1 inhibitor, or a pharmaceutically acceptable salt thereof, or a prodrug of the GlyT1 inhibitor or its pharmaceutically acceptable salt, wherein the porphyrin or porphyrin precursor is selected from the group consisting of 5-ALA; PBG; Hydroxymethylbilane; ZPPIX; Uroporphyrinogen I; Uroporphyrinogen III; Heptacarboxyporphyrinogen I; Heptacarboxyporphyrinogen III; Hexacarboxyporphyrinogen I; Hexacarboxyporphyrinogen III; Pentacarboxyporphyrinogen I; Pentacarboxyporphyrinogen III; Coproporphyrinogen I; Coproporphyrinogen III; Isocoproporphyrin; Porphobilinogen; and Protoporphyrinogen IX.

In some embodiments, the accumulation of one or more heme intermediates is inhibited, and wherein the one or more heme intermediates are selected from the group consisting of 5-ALA, coproporphyrin III, zinc-protoporphyrin IX (ZPPIX), porphobilinogen, uroporphyrin III, heptacarboxyl-porphyrin, and isocoproporphyrin. In some embodiments, the accumulation of one or more heme intermediates is inhibited, and wherein the one or more heme intermediates are selected from the group consisting of 5-ALA; PBG; Hydroxymethylbilane; ZPPIX; Uroporphyrinogen I; Uroporphyrinogen III; Heptacarboxyporphyrinogen I; Heptacarboxyporphyrinogen III; Hexacarboxyporphyrinogen I; Hexacarboxyporphyrinogen III; Pentacarboxyporphyrinogen I; Pentacarboxyporphyrinogen III; Coproporphyrinogen I; Coproporphyrinogen III; Isocoproporphyrin; Porphobilinogen; and Protoporphyrinogen IX. In some embodiments, the accumulation of the one or more heme intermediates is inhibited in a dose dependent manner. In some embodiments, the GlyT1 inhibitor demonstrates an EC50 of less than 500 nM. In some embodiments, the GlyT1 inhibitor demonstrates an EC50 of less than 100 nM.

In some embodiments, the subject has or is at risk for developing a hepatic porphyria and suffers from pain (e.g., neuropathic pain, e.g., chronic neuropathic pain) or neuropathy (e.g., progressive neuropathy). In some embodiments, the subject has an elevated level of ALA and/or PBG and suffers from chronic pain. In some embodiments, the subject has 5-ALA levels that are at least 10%, 20%, 30%, 40%, or 50% more than 5-ALA levels in a healthy subject prior to administration of the GlyT1 inhibitor. In some embodiments, the subject has HMB levels that are at least 10%, 20%, 30%, 40%, or 50% more than HMB levels in a healthy subject prior to administration of the GlyT1 inhibitor. In some embodiments, the subject has coproporphyrin III levels that are at least 10%, 20%, 30%, 40%, or 50% more than coproporphyrin III levels in a healthy subject prior to administration of the GlyT1 inhibitor. In some embodiments, the subject has ZPPIX levels that are at least 10%, 20%, 30%, 40%, or 50% more than ZPPIX levels in a healthy subject prior to administration of the GlyT1 inhibitor. In some embodiments, the subject has porphobilinogen levels that are at least 10%, 20%, 30%, 40%, or 50% more than porphobilinogen levels in a healthy subject prior to administration of the GlyT1 inhibitor. In some embodiments, the subject has uroporphyrin III levels that are at least 10%, 20%, 30%, 40%, or 50% more than uroporphyrin III levels in a healthy subject prior to administration of the GlyT1 inhibitor. In some embodiments, the subject has heptacarboxyl-porphyrin levels that are at least 10%, 20%, 30%, 40%, or 50% more than heptacarboxyl-porphyrin levels in a healthy subject prior to administration of the GlyT1 inhibitor. In some embodiments, the subject has isocoproporphyrin levels that are at least 10%, 20%, 30%, 40%, or 50% more than isocoproporphyrin levels in a healthy subject prior to administration of the GlyT1 inhibitor. In some embodiments, the subject's heme levels are substantially maintained during treatment. In some embodiments, the treatment decreases subject's heme levels decrease no more than 10% (e.g., 10%, 15%, 20%, 25%, and 30%). In some embodiments, the dosage of the pharmaceutical composition does not cause a substantial reduction in heme levels. In some embodiments, the subject has increased 5-ALA levels. In some embodiments, the subject has increased 5-ALA levels in the urine. In some embodiments, the subject has increased 5-ALA levels in the plasma. In some embodiments, the subject has increased HMB levels. In some embodiments, the subject has increased coproporphyrin III levels. In some embodiments, the subject has increased coproporphyrin III levels in the urine. In some embodiments, the subject has increased coproporphyrin III levels in the stool. In some embodiments, the subject has increased porphobilinogen (PBG) levels. In some embodiments, the subject has increased porphobilinogen (PBG) levels in the urine. In some embodiments, the subject has a plasma level or a urine level of 5-ALA or PBG that is greater than a reference value. In some embodiments, the reference value is two standard deviations above the mean level in a sample of healthy individuals. In some embodiments, the subject has a plasma level or a urine level of 5-ALA or PBG that is greater than or equal to 2 times, 3 times, 4 times, or 5 times that of an upper reference limit. In some embodiments, the subject has a urine level of PBG that is greater than or equal to 4.8 mmol/mol creatinine. In some embodiments, the subject has a plasma PBG level of greater than or equal to 0.12 μmol/L. In some embodiments, the subject has a urine PBG level of greater than or equal to 1.2 mmol/mol creatinine. In some embodiments, the subject has a plasma 5-ALA level of greater than or equal to 0.12 μmol/L. In some embodiments, the subject has a urine 5-ALA level of greater than or equal to 3.1 mmol/mol creatinine. In some embodiments, the method decreases the elevated level of 5-ALA and/or PBG. In some embodiments, the subject has increased uroporphyrin III levels. In some embodiments, the subject has increased uroporphyrin III levels in the urine. In some embodiments, the subject has an increased proportion of protoporphyrin to coproporphyrin in the stool. In some embodiments, the subject has increased heptacarboxyl-porphyrin levels. In some embodiments, the subject has increased heptacarboxyl-porphyrin levels in the urine. In some embodiments, the subject has increased heptacarboxyl-porphyrin levels in the stool. In some embodiments, the subject has increased isocoproporphyrin levels. In some embodiments, the subject has increased isocoproporphyrin levels in the stool. In some embodiments, the subject has increased ZPPIX levels in erythrocytes.

In some embodiments, the method decreases 5-ALA levels in the subject. In some embodiments, the method decreases 5-ALA levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases HMB levels in the subject. In some embodiments, the method decreases HMB levels in the subject by at least 10% (e.g., 10%, 15%, 20%, 25%, 30%, 35%, 40%, 45%, 50%, 55% 60%, 65%, 70%, 75%, 80%, 85%, 90%, 95%, or at least 100%). In some embodiments, the method decreases coproporphyrin III levels in the subject. In some embodiments, the method decreases coproporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases PBG levels in the subject. In some embodiments, the method decreases PBG levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method is effective to decrease the level of 5-ALA and/or PBG. In some embodiments, the level of 5-ALA and/or PBG is decreased such that it falls below a reference value. In some embodiments, the reference value is an upper reference limit. In some embodiments, the method decreases uroporphyrin III levels in the subject. In some embodiments, the method decreases uroporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases the proportion of protoporphyrin to coproporphyrin in the subject. In some embodiments, the method decreases the proportion of protoporphyrin to coproporphyrin in the subject by at least 10% (e.g., 10%, 15%, 20%, 25%, 30%, 35%, 40%, 45%, 50%, 55% 60%, 65%, 70%, 75%, 80%, 85%, 90%, 95%, or at least 100%). In some embodiments, the method decreases heptacarboxyl-porphyrin levels in the subject. In some embodiments, the method decreases heptacarboxyl-porphyrin levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases isocoproporphyrin levels in the subject. In some embodiments, the method decreases isocoproporphyrin levels in the subject by at least 10% (e.g., 10%, 15%, 20%, 25%, 30%, 35%, 40%, 45%, 50%, 55% 60%, 65%, 70%, 75%, 80%, 85%, 90%, 95%, or at least 100%). In some embodiments, the method decreases ZPPIX levels in the subject. In some embodiments, the method decreases ZPPIX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In some embodiments, the subject's plasma porphyrin fluoresces at a peak between 615 nm and 620 nm when illuminated with blue light (e.g., 400-420 nm light). In some embodiments, the subject's plasma porphyrin fluoresces at a peak between 624 nm and 627 nm when illuminated with blue light (e.g., 400-420 nm light). In some embodiments, the subject's skin porphyrin fluoresces at a peak between 615 nm and 620 nm when illuminated with blue light (e.g., 400-420 nm light). In some embodiments, the subject's skin porphyrin fluoresces at a peak between 624 nm and 627 nm when illuminated with blue light (e.g., 400-420 nm light). In some embodiments, the subject has a defect in an enzyme selected from the group consisting of ALA-dehydratase; PBG deaminase; Uroporphyrinogen III synthase; Uroporphyrinogen decarboxylase; Coproporphyrinogen oxidase; and Protoporphyrinogen oxidase. In some embodiments, the subject has mutation in a gene selected from the group consisting of ALAD; HMBS; UROS; UROD; CPOX; and PPOX.

In some embodiments, the GlyT1 inhibitor is administered after an acute attack. In some embodiments, the GlyT1 inhibitor is administered during an acute attack. In some embodiments, the GlyT1 inhibitor is administered during a prodrome. In some embodiments, the prodrome is characterized by pain (e.g., headache and/or abdominal pain), nausea, psychological symptoms (e.g., anxiety), restlessness and/or insomnia. In some embodiments, the GlyT1 inhibitor is administered prophylactically to prevent an acute attack of hepatic porphyria. In some embodiments, the GlyT1 inhibitor is administered during a particular phase of the menstrual cycle, e.g., during the luteal phase. In some embodiments, the GlyT1 inhibitor ameliorates or prevents cyclical attacks of hepatic porphyria. In some embodiments, the cyclical attacks are associated with a precipitating factor. In some embodiments, the precipitating factor is a particular phase of the menstrual cycle, e.g., the luteal phase. In some embodiments, the precipitating factor is the premenstrual phase. In some embodiments, the precipitating factor is exposure to a chemical. In some embodiments, the precipitating factor is exposure to lead. In some embodiments, the precipitating factor is selected from the group consisting of drugs, xenobiotics, steroid hormones, smoking, alcohol, decreased intake of calories or carbohydrates, fasting, metabolic stress, and psychological stress. In some embodiments, the method decreases pain or neuropathy. In some embodiments, the method prevents acute attacks of hepatic porphyria. In some embodiments, the method decreases or prevents nerve damage. In some embodiments, the GlyT1 inhibitor is administered prophylactically beginning at puberty. In some embodiments, the method further comprises administering to the subject an additional active agent and/or supportive therapy. In some embodiments, the additional active agent and/or supportive therapy is selected from the group consisting of: avoiding sunlight, topical sunscreens, skin protection, UVB phototherapy, Afamelanotide (Scenesse®), bortezomib, heme infusions, sufficient caloric support, Givosiran, RNAi mediated silencing of various enzymes (e.g., ALA synthase), avoiding precipitating factors, 4-aminoquinolines, chloroquine, hydroxychloroquine, phlebotomy, intravenous magnesium, LH-RH agonists, enzyme replacement therapy (e.g., recombinant human PBGD), gene therapy (e.g., transfer of PBGD gene in liver cells by viral vectors), hemodialysis, pharmacologic chaperone treatment, proteasome inhibitors, chemical chaperones, cholestyramine, activated charcoal, iron supplementation, liver transplantation, bone marrow transplantation, splenectomy, and blood transfusion.

In certain embodiments, the GlyT1 inhibitor is a compound of Formula I,

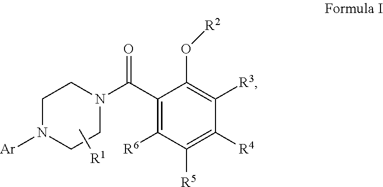

wherein Ar is unsubstituted or substituted aryl or 6-membered heteroaryl containing one, two or three nitrogen atoms, wherein the substituted aryl and the substituted heteroaryl groups are substituted by one or more substituents selected from the group consisting of hydroxy, halogen, NO₂, CN, (C₁-C₆)-alkyl, (C₁-C₆)-alkyl substituted by halogen, (C₁-C₆)-alkyl substituted by hydroxy, (CH₂)n-(C₁-C₆)-alkoxy, (C₁-C₆)-alkoxy substituted by halogen, NR⁷R⁸, C(O)R⁹, SO₂R¹⁰, and —C(CH₃)=NOR⁷, or are substituted by a 5-membered aromatic heterocycle containing 1-4 heteroatoms selected from N and O, which is optionally substituted by (C₁-C₆)-alkyl; R¹ is hydrogen or (C₁-C₆)-alkyl; R² is hydrogen, (C₁-C₆)-alkyl, (C₂-C₆)-alkenyl, (C₁-C₆)-alkyl substituted by halogen, (C₁-C₆)-alkyl substituted by hydroxy, (CH2)n-(C₃-C₇)-cycloalkyl optionally substituted by (C₁-C₆)-alkoxy or by halogen, CH(CH₃)—(C₃-C₇)-cycloalkyl, (CH₂)ₙ₊₁—C(O)—R⁹, (CH₂)ₙ₊₁—CN, bicyclo[2.2.1]heptyl, (CH₂)ₙ₊₁—O—(C₁-C₆)-alkyl, (CH₂)ₙ-heterocycloalkyl, (CH₂)ₙ-aryl or (CH₂)ₙ-5 or 6-membered heteroaryl containing one, two or three heteroatoms selected from the group consisting of oxygen, sulphur or nitrogen wherein aryl, heterocycloalkyl and heteroaryl are unsubstituted or substituted by one or more substituents selected from the group consisting of hydroxy, halogen, (C₁-C₆)-alkyl and (C₁-C₆)-alkoxy; R³, R⁴ and R⁶ are each independently hydrogen, hydroxy, halogen, (C₁-C₆)-alkyl, (C₁-C₆)-alkoxy or O—(C₃-C₆)-cycloalkyl; R⁵ is NO₂, CN, C(O)R⁹ or SO₂R¹⁰; R⁷ and R⁸ are each independently hydrogen or (C₁-C₆)-alkyl; R⁹ is hydrogen, (C₁-C₆)-alkyl, (C₁-C₆)-alkoxy or NR⁷R⁸; R¹⁰ is (C₁-C₆)-alkyl optionally substituted by halogen, (CH₂)ₙ—(C₃-C₆)-cycloalkyl, (CH₂)ₙ—(C₃-C₆)-alkoxy, (CH₂)ₙ-heterocycloalkyl or NR⁷R⁸; n is 0, 1, or 2; or a pharmaceutically acceptable salt thereof, or a prodrug of the compound or its pharmaceutically acceptable salt.

In certain embodiments, GlyT1 inhibitor is a compound having a formula of

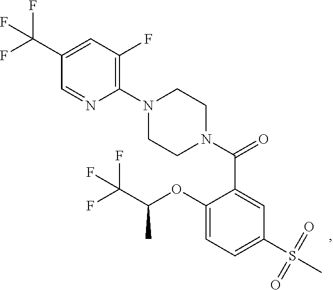 [SMILES: C[C@H](Oc1ccc(S(C)(=O)=O)cc1C(=O)N1CCN(c2ncc(C(F)(F)F)cc2F)CC1)C(F)(F)F]

bitopertin, or a pharmaceutically acceptable salt thereof, or a prodrug of the compound or its pharmaceutically acceptable salt.

In certain embodiments, the GlyT1 inhibitor is a compound of Formula II,

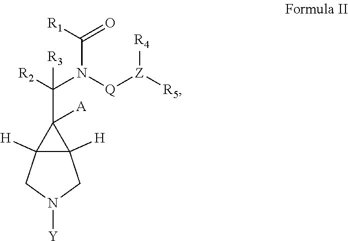

wherein R₁ represents a heteroaryl selected from the group consisting of: imidazolyl, thiazolyl, pyridyl, oxazolyl, pyrazolyl, triazolyl, oxadiazolyl, quinolinyl, isoxazolyl, pyrroloimidazoyl, and thiadiazole, wherein said heteroaryl is optionally substituted by one or more substituents selected from —OH, —NR₇R₈, halogen, (C₁-C₈)alkyl, (C₃-C₁₀)cycloalkyl, (C₁-C₈)alkoxy, (C₁-C₁₂)alkoxyalkyl, (C₁-C₈)hydroxyalkyl, (C₆-C₁₄)aryl and benzyl; R₂, R₃ and A independently represent H or (C₁-C₈)alkoxy, wherein said alkyl is optionally substituted by one or more —OH, (C₁-C₈)alkoxy, —NR₇R₈ or halogen; Q represents —(CH₂)ₙ—, where n=1, 2, 3 or 4 or —(CH₂)ₘ—O—, where m=2, 3 or 4; Z represents (C₆-C₁₄)aryl, (C₁-C₈)alkyl or (C₃-C₈)cycloalkyl; R₄ and R₅ each independently represent H, halogen, (C₁-C₈)alkyl, (C₆-C₁₄)aryl, (C₆-C₁₄)aryloxy, (C₁-C₈)alkoxy, (3-10 membered)heterocycloalkyl or (C₃-C₈)cycloalkoxy; wherein R₄ and R₅ are optionally substituted by one or more —OH, (C₁-C₈)alkoxy, —NR₇R₈ or halogen; Y represents —R₆, —(CH₂)o-R₆, —C(R₆)₃ or —CH(R₆)₂, wherein 0=1, 2 or 3; R₆ represents H, (C₆-C₁₄)aryl, (C₁₋₁₀)alkyl, (C₃-C₁₀)cycloalkyl, (C₅-C₁₈)bicycloalkyl, (C₅-C₁₈)tricycloalkyl, (3-10 membered)heterocycloalkyl, (5-10 membered)heteroaryl, —C(═O)NR₇R₈, or —C(═O)OR₇, wherein said R₆ groups can optionally be substituted with one or more X groups; wherein X=—OH, (C₁-C₈)alkoxy, —NR₁₁R₁₂, —SO₂R₁₀, —C(═O)R₁₀, halogen, cyano, (C₁-C₈)alkyl, (C₁-C₁₀)alkoxyalkyl, (5-10 membered)heteroaryl, (C₆-C₁₄)aryl, (C₆-C₁₄)aryloxy, benzyl, or (C₁-C₈)hydroxyalkyl; wherein R₇ and R₈ independently represent H, (C₁-C₈)alkyl, (C₃-C₈)cycloalkyl, (5-10 membered)heterocycloalkyl, (C₁-C₈)hydroxyalky, (5-10 membered)heteroaryl or (C₁-C₁₀)alkoxyalkyl; wherein R₇ and R₈ may optionally be substituted by one or more X groups; or R₇ and R₈ together with the nitrogen in which they may be attached may form a (3-10 membered)heterocycloalkyl group optionally substituted by one or more X groups; wherein R₁₀ represents (C₁-C₈)alkyl, (C₃-C₈)cycloalkyl, (3-10 membered)heterocycloalkyl, (C₁-C₈)hydroxyalky, (5-10 membered)heteroaryl or (C₁-C₁₀)alkoxyalkyl; wherein R₁₁ and R₁₂ independently represent H, (C₁-C₈)alkyl, (C₃-C₈)cycloalkyl, (5-10 membered)heterocycloalkyl, (C₁-C₈)hydroxyalky, (5-10 membered)heteroaryl or (C₁-C₁₀)alkoxyalkyl; or a pharmaceutically acceptable salt thereof, or a prodrug of the compound or its pharmaceutically acceptable salt. In certain such embodiments, the GlyT1 inhibitor is a compound having a formula of

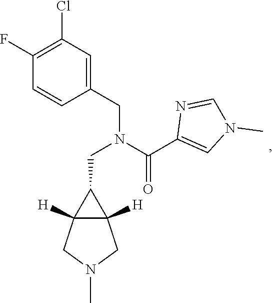 [SMILES: CN1C[C@@H]2[C@H](C1)[C@@H]2CN(Cc1ccc(F)c(Cl)c1)C(=O)c1cn(C)cn1]

or a pharmaceutically acceptable salt thereof, or a prodrug of the compound or its pharmaceutically acceptable salt. In other such embodiments, the GlyT1 inhibitor is a compound having a formula of

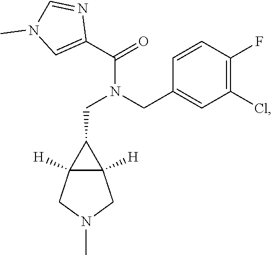 [SMILES: CN1C[C@@H]2[C@H](C1)[C@H]2CN(Cc1ccc(F)c(Cl)c1)C(=O)c1cn(C)cn1]

PF-3463275, or a pharmaceutically acceptable salt thereof, or a prodrug of the compound or its pharmaceutically acceptable salt.

In certain embodiments, the GlyT1 inhibitor is a compound of Formula III,

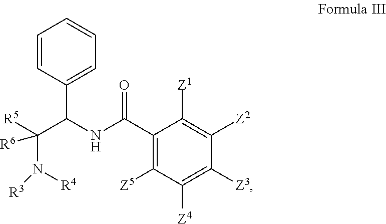

wherein Z¹ is selected from the group consisting of C₁₋₄alkyl, C₃₋₆CycloalkVI, C₁₋₄alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro; Z² is selected from the group consisting of hydrogen, halogen, cyano, C₁₋₄alkyl, phenyl, haloC₁₋₄alkyl, haloC₁₋₄alkoxy, halophenyl, C₁₋₄alkoxyC₁₋₄alkyl and C₃₋₆cycloalkyl; Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl; Z⁴ is selected from the group consisting of hydrogen, halogen, C₁₋₃alkyl, haloC₁₋₄alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl; Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl; whereby if more than one of Z¹ to Z⁵ is methoxy, then only Z¹ and Z⁵ are methoxy R³ and R⁴ are independently selected from hydrogen and C₁₋₄alkyl, optionally substituted with one or more groups Y; or R³ and R4 together with the nitrogen atom to which they are attached form a saturated or partially unsaturated A-, 5-6- or 7-membered carbocyclic ring optionally substituted with a group Y′; Y is selected from the group consisting of C₁₋₄alkoxy, hydroxy, haloC₁₋₄alkoxy and C₃₋₅cycloalkyl; Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH2- or —CH2-CH2- bridge between two atoms on the A-, 5-, 6- or 7-membered carbocyclic ring; R⁵ and R⁶ are independently C₁₋₄alkyl, optionally substituted with one or more groups X; or R⁵ and R⁶ together with the carbon atom to which they are attached form a saturated 5- or 6-membered ring carbocyclic optionally substituted with one or more groups X′, in the case of R⁵ and R6 together with the carbon atom to which they are attached forming a 5-membered saturated carbocyclic ring, that ring may optionally further comprising an additional heteroatom group selected from O, N and S(O) m, where m=0, 1 or 2; X is selected from the group consisting of halogen, hydroxy, C₁₋₄alkoxy, haloC₁₋₄alkyl, haloC₁₋₄alkoxy and C₅₋₁₀aryl; and X′ is selected from the group consisting of halogen, hydroxy, C₁₋₄alkyl, C₁₋₄ alkoxy, haloC₁₋₄alkyl, haloC₁₋₄alkoxy and C₅₋₁₀aryl; whereby R³, R⁴, R⁵ and R⁶ are not all simultaneously unsubstituted methyl; with the provisos that when simultaneously Z¹ is propyloxy, Z³ is chloro, Z²=Z⁴=Z⁵=H, and R⁵ and R⁶ are both methyl, then R³ and R⁴ together with the nitrogen atom to which they are attached do not form a 2-methylpyrrolidine group; when simultaneously Z¹ is methyl, Z³ is methoxy, Z²=Z4=Z5=H, and R⁵ and R⁶ are both methyl, then R³ and R⁴ together with the nitrogen atom to which they are attached do not form a pyrrolidine group, or a pharmaceutically acceptable salt thereof, or a prodrug of the compound or its pharmaceutically acceptable salt. In certain such embodiments, the GlyT1 inhibitor is a compound having a formula of

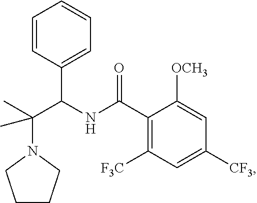 [SMILES: COc1cc(C(F)(F)F)cc(C(F)(F)F)c1C(=O)NC(c1ccccc1)C(C)(C)N1CCCC1]

or a pharmaceutically acceptable salt thereof, or a prodrug of the compound or its pharmaceutically acceptable salt.

In certain embodiments, the GlyT1 inhibitor is a compound of Formula IV,

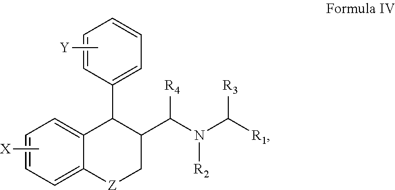

wherein Z is (CH₂)ₙ, O, S, SO, SO₂ or N—R₅; n is 0, 1 or 2; X represents 1-3 substituents independently selected from hydrogen, halogen, (C₁₋₆)alkyioxy, (C₃₋₆)cycloalkyloxy, (C₆₋₁₂)aryloxy, (C₆₋₁₂)aryl, thienyl, SR₆, SOR₆, SO₂R₆, NR₆R₆, NHR₆, NH₂, NHCOR₆, NSO₂R₆, CN, COOR₆ and (C₁₋₄)alkyl, optionally substituted with halogen, (C₆₋₁₂)aryl, (C₁₋₆)alkyloxy or (C₆₋₁₂)aryloxy; or 2 substituents at adjacent positions together represent a fused (C₅₋₆)aryl group, a fused (C₅₋₆)cycloalkyl ring or 0-(CH₂)ₘ—O; m is 1 or 2; Y represents 1-3 substituents independently selected from hydrogen, halogen, (C₁₋₄)alkyloxy, SR₆, NR₆R₆ and (C₁₋₄)alkyl, optionally substituted with halogen; R₁ is COOR₇ or CONR₈R₉; R₂ and R₆ are (C₁₋₄)alkyl; R₃, R₄ are R₅ are independently hydrogen or (C₁₋₄)alkyl; R₇, R₈ and R₉ are independently hydrogen, (C₁₋₄)alkyl, (C₆₋₁₂)aryl or arylalkyl, or a pharmaceutically acceptable salt thereof, or a prodrug of the compound or its pharmaceutically acceptable salt. In certain such embodiments, the GlyT1 inhibitor is a compound having a formula of

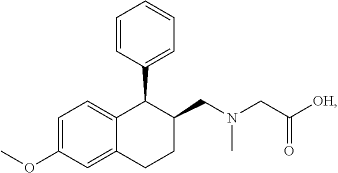 [SMILES: COc1ccc2c(c1)CC[C@H](CN(C)CC(=O)O)[C@@H]2c1ccccc1]

or a pharmaceutically acceptable salt thereof, or a prodrug of the compound or its pharmaceutically acceptable salt.

In certain embodiments, the GlyT1 inhibitor is a compound of Formula V,

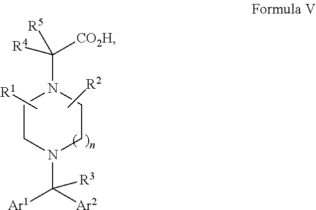

wherein n is an integer from 1 to 3; R¹ and R² are independently selected from hydrogen, alkyl, haloalkyl, alkoxy, haloalkoxy, aryl, heteroaryl, cycloalkyl, or heterocyclyl wherein the aforementioned rings are optionally substituted with R^{a}, R^{b}, or R^{c} independently selected from alkyl, halo, haloalkyl, alkoxy, haloalkoxy, hydroxy, cyano, monosubstituted amino, or disubstituted amino; or R¹ and R², when attached to the same carbon atom, can combine to form cycloalkyl or monocyclic saturated heterocyclyl to give a spiro ring wherein the cycloalkyl or monocyclic saturated heterocyclyl can be optionally substituted with R^{d}, R^{c}, or R^{f} independently selected from alkyl, alkoxy, fluoro, fluoroalkyl, fluoroalkoxy, hydroxy, monosubstituted amino, or disubstituted amino; or R¹ and R², when attached to carbon atoms 2 and 5 or 3 and 6 positions of the piperazine ring, can combine to form —C₁-C₃-alkylene chain wherein one of the carbon atoms in the alkylene chain is optionally replaced by a —NR—, —O—, —S(O)n- (where R is hydrogen or alkyl and n is 0-2) and further wherein one or two hydrogen atoms in the alkylene chain can be optionally substituted with one or two alkyl; R³, R⁴ and R⁵ are independently hydrogen, alkyl, fluoro, or fluoroalkyl; and Ar¹ and Ar² are independently aryl, heteroaryl, cycloalkyl, or heterocyclyl where each of the aforementioned ring is optionally substituted with R^{g}, R^{h} or Rⁱ where R^{g} is alkyl, —C═C— R⁶ (where R⁶ is aryl or heteroaryl), halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{h} and Rⁱ are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl., heteroaryl, cycloalkyl, or heterocyclyl where the aromatic or alicyclic ring-in R^{g}, R^{h} and Rⁱ is optionally substituted with R^{j}, R^{k}, or R^{l} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or a pharmaceutically acceptable salt thereof provided that: the compound of Formula V is not 2-(4-benzhydrylpiperazin-1-yl)acetic acid, 2-(4-((4-chlorophenyl)(phenyl)methyl)piperazin-1-yl)acetic acid, 2-((2R,5S)-4-((R)-(4-(1H-tetrazol-5-yl)phenyl)(3-hydroxyphenyl)methyl)-2,5-dimethylpiperazin-1-yl)acetic acid, or 2-((2R,5S)-4-((R)-(4-cyanophenyl)(3-hydroxyphenyl)methyl)-2,5-dimethylpiperazin-1-yl)acetic acid, or a pharmaceutically acceptable salt thereof, or a prodrug of the compound or its pharmaceutically acceptable salt. In certain such embodiments, the GlyT1 inhibitor is a compound having a formula of

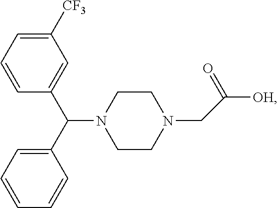 [SMILES: O=C(O)CN1CCN(C(c2ccccc2)c2cccc(C(F)(F)F)c2)CC1]

or a pharmaceutically acceptable salt thereof, or a prodrug of the compound or its pharmaceutically acceptable salt.

In certain embodiments, the GlyT1 inhibitor is a compound of Formula VI,

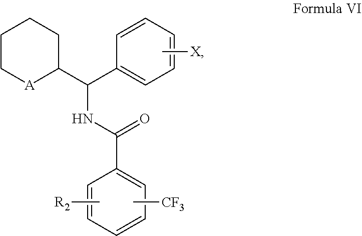

wherein A represents a group of general formula N—R₁, a group of general formula N+(O—)R₁ or a group of general formula N+(R′)R₁, and in which R₁ represents either a hydrogen atom, or a linear or branched (C₁-C₇)alkyl group optionally substituted with one or more fluorine atoms, or a (C₄-C₇)cycloalkyl group, or a (C₃-C₇)cycloalkyl(C₁-C₃)alkyl group, or a phenyl(C₁-C₃)alkyl group optionally substituted with one or two hydroxyl or methoxy groups, or a (C₂-C₄)alkenyl group, or a (C₂-C₄)alkynyl group; R′ represents a linear or branched (C₁-C₇)alkyl group; X represents a hydrogen atom or one or more substituents chosen from halogen atoms and trifluoromethyl, linear or branched (C₁-C₄)alkyl and (C₁-C₄)alkoxy groups; R₂ represents either a hydrogen atom, or one or more substituents chosen from halogen atoms and trifluoromethyl, (C₁-C₄)alkyl or (C₁-C₄)alkoxy groups, or amino groups of general formula NR₃R₄ in which R₃ and R₄ each represent, independently of each other, a hydrogen atom or a (C₁-C₄)alkyl group, or form with the nitrogen atom carrying them a pyrrolidine, piperidine or morpholine ring, or a phenyl group optionally substituted with an atom or a group as defined for the symbol X above, or a pharmaceutically acceptable salt thereof, or a prodrug of the compound or its pharmaceutically acceptable salt. In certain such embodiments, the GlyT1 inhibitor is a compound having a formula of

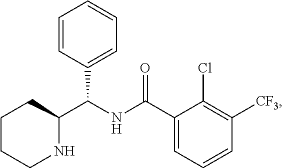 [SMILES: O=C(N[C@@H](c1ccccc1)[C@@H]1CCCCN1)c1cccc(C(F)(F)F)c1Cl]

or a pharmaceutically acceptable salt thereof, or a prodrug of the compound or its pharmaceutically acceptable salt.

In certain embodiments, the GlyT1 inhibitor is a compound of Formula VII,

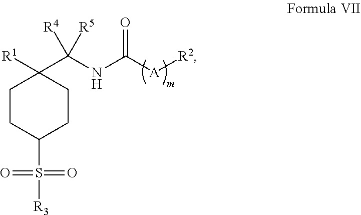

wherein R¹ is —(CH₂)ₙ—R^{1a}, wherein n is independently 0-6, and R^{1a} is selected from the group consisting of: (1) C₁₋₆alkyl, which is unsubstituted or substituted with 1-6 halogen, hydroxy, (2) phenyl substituted with R^{2a}, R^{2b} and R^{2c}, (3) C₃₋₆cycloallyl, which is unsubstituted or substituted with C₁₋₆alkyl, 1-6 halogen, hydroxy or —NR¹⁰R¹¹, (4) —O—C₁₋₆alkyl, which is unsubstituted or substituted with 1-6 halogen, hydroxy or —NR¹⁰R¹¹, (5) —CO2R⁹, wherein R9 is independently selected from: (a) hydrogen, (b) —C₁₋₆alkyl, which is unsubstituted or substituted with 1-6 fluoro, (c) benzyl, and (d) phenyl, (6) —NR¹⁰R¹¹, wherein R¹⁰ and R¹¹ are independently selected from: (a) hydrogen, (b) —C₁₋₆alkyl, which is unsubstituted or substituted with hydroxy, 1-6 fluoro or —NR¹²R¹³, where R¹² and R¹³ are independently selected from hydrogen and —C₁₋₆alkyl, (c) —C₃₋₆cycloalkyl, which is unsubstituted or substituted with hydroxy, 1-6 fluoro or —NR¹²R¹³, (d) benzyl, (e) phenyl, and (7) —CONR¹⁰R¹¹; R2 is selected from the group consisting of: (1) phenyl, which is substituted with R^{2a}, R^{2b} and R^{2c}, (2) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c}, (3) C₃₋₆cycloalkyl, which is unsubstituted or substituted with 1-6 halogen, hydroxy or —NR¹⁰R¹¹, and (4) —C₁₋₆alkyl-(C₃₋₆cycloalkyl), which is unsubstituted or substituted with 1-6 halogen, hydroxy or —NR¹⁰R¹¹; R^{2a}, R^{2b} and R^{2c} are independently selected from the group consisting of: (1) hydrogen, (2) halogen, (3) —C₁₋₆alkyl, which is unsubstituted or substituted with: (a) 1-6 halogen, (b) phenyl, (c) C₃₋₆cycloalkyl, or (d) —NR¹⁰R¹¹, (4) —O—C₁₋₆alkyl, which is unsubstituted or substituted with 1-6 halogen, (5) hydroxy, (6) —SCF₃, (7) —SCHF₂, (8) —SCH₃, (9) —CO₂R⁹, (10) —CN, (11) —SO₂R⁹, (12) —SO₂—NR¹⁰R¹¹, (13) —NR¹⁰R¹¹, (14) —CONR¹⁰R¹¹, and (15) —NO₂; R³ is selected from the group consisting of: (1) C₁₋₆alkyl, which is unsubstituted or substituted with 1-6 halogen, hydroxyl, or —NR¹⁰R¹¹, (2) C₃₋₆cycloalkyl, which is unsubstituted or substituted with 1-6 halogen, hydroxyl or —NR¹⁰R¹¹, R⁴ and R⁵ are independently selected from the group consisting of: (1) hydrogen, and (2) C₁₋₆alkyl, which is unsubstituted or substituted with halogen or hydroxyl, or R⁴ and R⁵ taken together form a C₃₋₆cycloalkyl ring; A is selected from the group consisting of: (1) —O—, and (2) —NR¹⁰—; m is zero or one, whereby when m is zero R² is attached directly to the carbonyl; and pharmaceutically acceptable salts thereof and individual enantiomers and diastereomers thereof, or a pharmaceutically acceptable salt thereof, or a prodrug of the compound or its pharmaceutically acceptable salt. In certain such embodiments, the GlyT1 inhibitor is a compound having a formula of

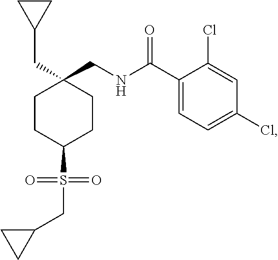 [SMILES: O=C(NC[C@]1(CC2CC2)CC[C@H](S(=O)(=O)CC2CC2)CC1)c1ccc(Cl)cc1Cl]

or a pharmaceutically acceptable salt thereof, or a prodrug of the compound or its pharmaceutically acceptable salt.

In certain embodiments, the GlyT1 inhibitor is a compound of Formula VIII,

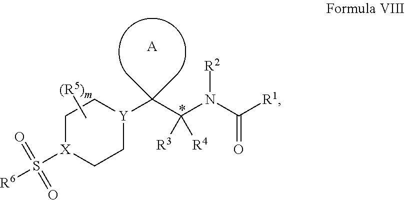

wherein R¹ is phenyl independently substituted from 1 to 5 times with halogen, C₁-C₃ alkyl, C₃-C₆ cycloalkyl, OR⁹, or SR¹⁰, wherein C₁-C₃ alkyl and C₃-C₆ cycloalkyl are optionally substituted with 1 to 10 times with R⁷; R² is H; R³ and R4 are each individually H or CH₃; R⁵ is selected from the group consisting of: (1) hydrogen, (2) C₁-C₆ alkyl which is optionally substituted from 1 to 11 times with R⁷, (3) gem-dialkyl, and (4) gem-dihalo; or two R⁵ substituents on the same carbon, together with the carbon atom to which they are attached, may form a 3-, 4-, or 5-membered cycloalkyl optionally substituted from 1 to 10 times with R⁷; or two R⁵ substituents on adjacent carbons of the ring to which they are attached, together may form a 3-, 4-, 5- or 6-membered cycloalkyl optionally substituted from 1 to 10 times with R⁷; R⁶ is

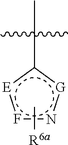

wherein E, F, and G are each independently nitrogen or carbon and R^{6a} is C₁-C₂ alkyl, which is optionally substituted 1 to 5 times with halogen or deuterium; R⁷ is selected from the group consisting of: (1) hydrogen, (2) halogen, (3) deuterium, (4) gem-dialkyl, (5) gem-dihalo, (6) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰, and (7) oxo or thio; R⁸ is selected from the group consisting of: (1) hydrogen, (2) halogen, (3) C₁-C₆ alkyl, C₂-C₆ alkenyl, C₂-C₆ alkynyl, C₃-C₇ cycloalkyl, or C₄-C₇ cycloalkylalkyl, wherein each of the C₁-C₆ alkyl, C₂-C₆ alkenyl, C₂-C₆ alkynyl, C₃-C₇ cycloalkyl, and C₄-C₇ cycloalkylalkyl is independently and optionally substituted from 1 to 11 times with R⁷, or (4) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰; R⁹ is selected from the group consisting of hydrogen, C₁-C₄ alkyl, C₃-C₇ cycloalkyl, C₄-C₇ cycloalkylalkyl, —C(O)NR¹¹R¹², and —C(O)ₚR¹⁰, wherein each of C₁-C₄ alkyl, C₃-C₇ cycloalkyl, and C₄-C₇ cycloalkylalkyl is optionally substituted from 1 to 11 times with R⁷; R¹⁰ is selected from the group consisting of hydrogen, C₁-C₄ alkyl, C₃-C₇ cycloalkyl C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R7 and aryl or heteroaryl is optionally substituted from 1 to 10 times with R⁸; R¹¹ and R¹² are each independently selected from the group consisting hydrogen, C₁-C₄ alkyl, C₃-C₇ cycloalkyl, C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R⁷ and aryl or heteroaryl is optionally substituted from 1 to 10 times with R⁸, or R¹¹ and R¹² are taken together with the nitrogen to which they are attached to form a saturated or partially saturated monocyclic or fused bicyclic heterocycle optionally substituted from 1 to 11 times with R⁷; A is

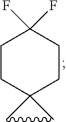

X is N; Y is N; p is 1, or 2; and m is 0; with the following provisos that: R⁶ cannot be (a) 1H-1,2,3-triazol-4-yl, or (b) 5-methylisoxazol-4-yl; or an oxide thereof, a pharmaceutically acceptable salt of the compound or its oxide, or an individual enantiomer or diastereomer thereof.

In certain embodiments, the GlyT1 inhibitor is a compound selected from any of the following:

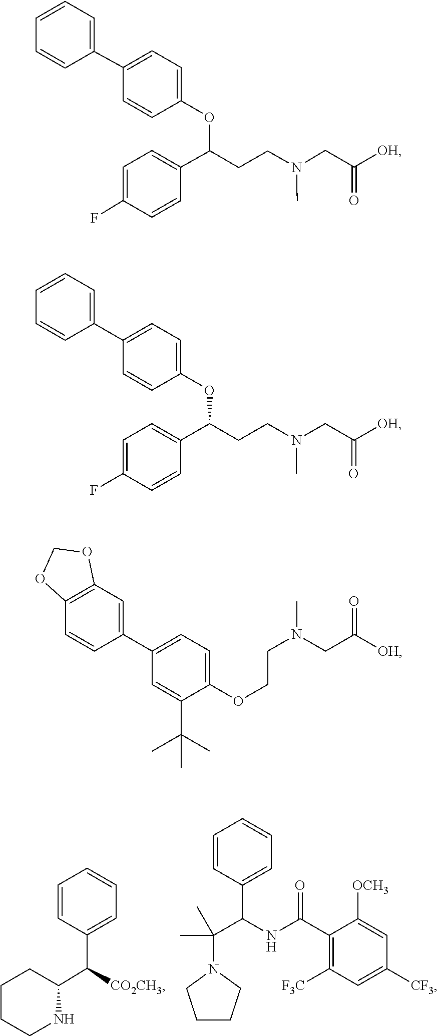 [SMILES: CN(CCC(Oc1ccc(-c2ccccc2)cc1)c1ccc(F)cc1)CC(=O)O ; CN(CC[C@@H](Oc1ccc(-c2ccccc2)cc1)c1ccc(F)cc1)CC(=O)O ; CN(CCOc1ccc(-c2ccc3c(c2)OCO3)cc1C(C)(C)C)CC(=O)O ; COC(=O)[C@H](c1ccccc1)[C@H]1CCCCN1 ; COc1cc(C(F)(F)F)cc(C(F)(F)F)c1C(=O)NC(c1ccccc1)C(C)(C)N1CCCC1]

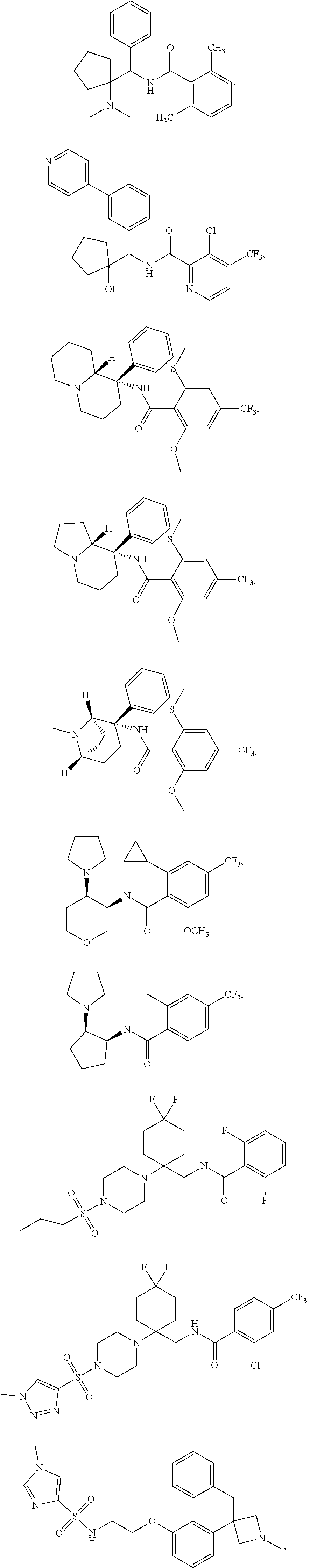

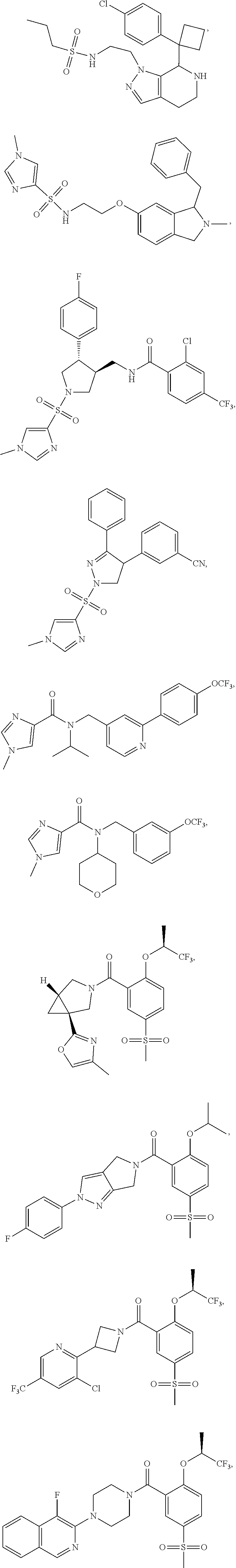

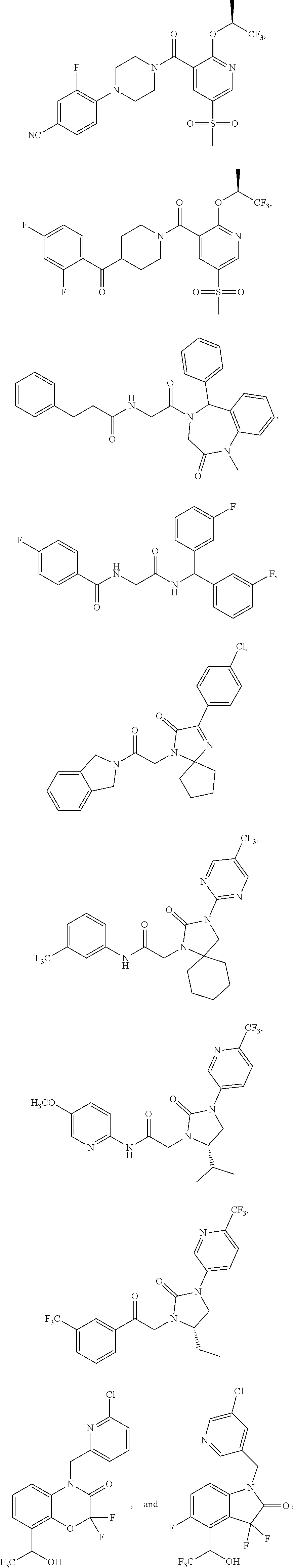

or a pharmaceutically acceptable salt thereof, or a prodrug of the compound or its pharmaceutically acceptable salt.

In certain embodiments, the GlyT1 inhibitor is a compound of formula IX,

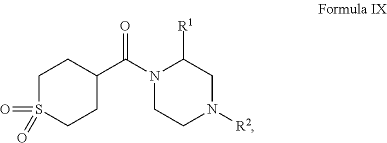

wherein R¹ represents phenyl or a 5 or 6 membered monocyclic heteroaryl having 1, 2, or 3 heteroatoms independently selected from O, N or S, wherein the phenyl or the heteroaryl is optionally substituted with one or more R³; R² represents aryl, a 5 or 6 membered monocyclic heteroaryl or a 8 to 10 membered bicyclic heteroaryl, the mono- or bicyclic heteroaryl having 1, 2, or 3 heteroatoms independently selected from O, N or S, wherein the aryl or the heteroaryl is optionally substituted with one or more R⁴; R³ is a halogen, a C₁₋₄-alkyl or a C₃₋₆-cycloalkyl, wherein the C₁₋₄-alkyl or the C₃₋₆-cycloalkyl is optionally substituted with one or more halogens; and R⁴ is a halogen, —CN, C₁₋₄-alkyl, C₃₋₆-cycloalkyl, —C₁₋₃-alkyl —C₃₋₆-cycloalkyl or —O—C₁₋₆ alkyl, wherein the C₁₋₄-alkyl, C₃₋₆-cycloalkyl, —C₁₋₃-alkyl —C₃₋₆-cycloalkyl or the —O—C₁₋₆-alkyl is optionally substituted with one or more halogens; or a pharmaceutically acceptable salt thereof, or a tautomer or stereoisomer of the compound or its pharmaceutically acceptable salt, or a mixture of any of the foregoing.

In certain embodiments, the GlyT1 inhibitor is a compound of formula X,

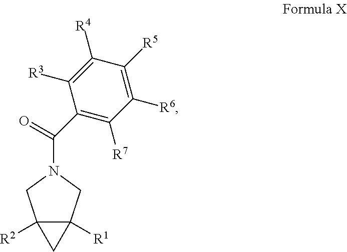

wherein R¹ is selected from the group consisting of a) 5 or 6 membered monocyclic heteroaryl, having 1, 2, 3 or 4 heteroatoms independently selected from the group consisting of O, N and S(O)r, b) 5 or 6 membered monocyclic partially saturated heterocycloalkyl, having 1, 2 or 3 heteroatoms independently selected from the group consisting of O, N and S(O)r, and c) 9 or 10 membered bicyclic heteroaryl, having 1, 2 or 3 heteroatoms independently selected from the group consisting of O, N and S(O)ᵣ, wherein r is 0, 1 or 2; wherein each of said groups a), b) and c) is optionally substituted with 1 or more substituents independently selected from the group consisting of C₁₋₄-alkyl-, C₁₋₄-alkyl-O—, oxetanyl, tetrahydrofuranyl, tetrahydropyranyl, C₃₋₆-cycloalkyl- and C₃₋₆-cycloalkyl-O— and in case a substituent is attached to a nitrogen ring atom said substituent is selected from the group consisting of C₁₋₄-alkyl-, C₁₋₄-alkyl-CO—, C₃₋₆-cycloalkyl- and C₃₋₆-cycloalkyl-CO—, and wherein each of said C₁₋₄-alkyl-, C₁₋₄-alkyl-O—, C₁₋₄-alkyl-CO—, oxetanyl, tetrahydrofuranyl, tetrahydropyranyl, C₃₋₆-cycloalkyl-, C₃₋₆-cycloalkyl-CO— or C₃₋₆-cycloalkyl-O— substituents may be substituted by 1 or more substituents independently selected from the group consisting of fluoro, —CF₃, —CHF₂, —CH₂F and —CN; R² is selected from the group consisting of hydrogen, C₁₋₄-alkyl-, C₁₋₄-alkyl-O—, —CN and C₃₋₆-cycloalkyl-, wherein each of said C₁₋₄-alkyl-, C₁₋₄-alkyl-O— and C₃₋₆-cycloalkyl-group may be optionally substituted with 1, 2, 3 or more substituents independently selected from the group consisting of fluoro, —CF₃, —CHF₂, —CH₂F and —CN; R³ is selected from the group consisting of C₁₋₆-alkyl-O—, C₃₋₆-cycloalkyl-O—, morpholino, pyrazolyl and a 4 to 7 membered, monocyclic heterocycloalkyl-O— with 1 oxygen atom as ring member and optionally 1 or 2 heteroatoms independently selected from the group consisting of O, N and S(O)ₛ with s=0, 1 or 2, wherein said C₁₋₆-alkyl-O— and said C₃₋₆-cycloalkyl-O— may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O— and C₃₋₆-cycloalkyl-O—; R⁴ is hydrogen; or R³ and R⁴ together with the ring atoms of the phenyl group to which they are bound may form a 4, 5 or 6 membered, monocyclic, partially saturated heterocycloalkyl or a heteroaryl each of which having 1, 2 or 3 heteroatoms independently selected from the group consisting of O, N and S(O)ₛ with s=0, 1 or 2, wherein there must be 1 ring oxygen atom that is directly attached to the ring carbon atom of said phenyl group to which R³ is attached to in general formula (I); wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O—, C₃₋₆-cycloalkyl-O—, oxetanyl-O—, tetrahydrofuranyl-O— and tetrahydropyranyl-O—; R⁵ is hydrogen; R⁶ is selected from the group consisting of hydrogen, C₁₋₄-alkyl-SO₂—, C₃₋₆-cycloalkyl-SO₂ and —CN; R⁷ is hydrogen; or one of the pairs a) R⁶ and R⁷ or b) R⁶ and R⁵ form together with the ring atoms of the phenyl group to which they are bound, a 5 or 6 membered, partially saturated monocyclic heterocycloalkyl group having 1, 2 or 3 heteroatoms independently selected from the group consisting of O, N and S(O)ᵤ with u=0, 1 or 2, wherein there must be 1 —SO₂-member that is directly attached to the ring carbon atom of said phenyl group to which R⁶ is attached to in general formula (I), wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₁₋₆-alkyl-O— and C₃₋₆-cycloalkyl-O— or a pharmaceutically acceptable salt thereof. In certain such embodiments, the GlyT1 inhibitor is a compound having a formula

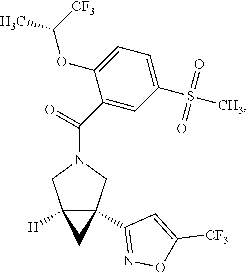 [SMILES: C[C@@H](Oc1ccc(S(C)(=O)=O)cc1C(=O)N1C[C@@H]2C[C@]2(c2cc(C(F)(F)F)on2)C1)C(F)(F)F]

or a pharmaceutically acceptable salt thereof.

In some embodiments, the GlyT1 inhibitor is a compound of Formula XI,

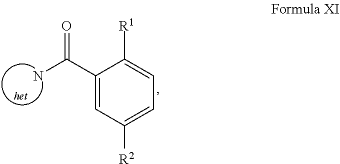

wherein R¹ is halogen, —OR^{1′}, —SR^{1″}, cycloalkyl, cyclic amide, heterocycloalkyl, aryl or 5- or 6-membered heteroaryl containing one, two or three heteroatoms selected from the group consisting of oxygen, sulphur and nitrogen; R^{1′} and R^{1″} are each independently hydrogen, lower alkyl, lower alkyl substituted by halogen, —(CH₂)ₓ-cycloalkyl or —(CH₂)ₓ-aryl; R² is —S(O)₂-lower alkyl, —S(O)₂NH-lower alkyl, NO₂ or CN;

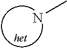

is an aromatic or partially aromatic bicyclic amine, having one or two additional N-atoms selected from the group consisting of

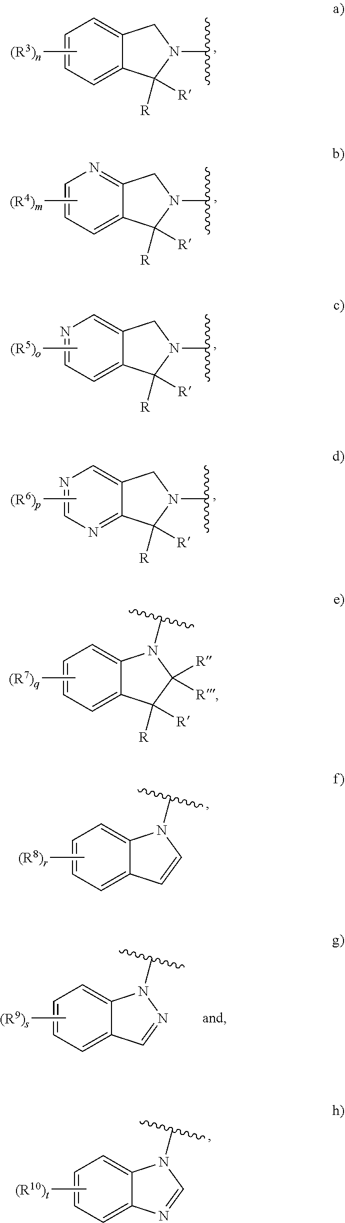

and wherein one of the additional N-ring atoms of the aromatic or partially aromatic bicyclic amine can be available in form of its oxide

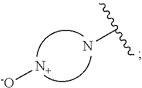

R3 to R₁₀ are each independently hydrogen, hydroxy, halogen, ═O, lower alkyl, cycloalkyl, heterocycloalkyl, lower alkoxy, CN, NO2, NH2, aryl, 5- or 6-membered heteroaryl containing one, two or three heteroatoms selected from the group consisting of oxygen, sulphur and nitrogen, —NH-lower alkyl, —N(lower alkyl)2, cyclic amide, —C(O)-cyclic amide, S-lower alkyl, —S(O)2-lower alkyl, lower alkyl substituted by halogen, lower alkoxy substituted by halogen, lower alkyl substituted by hydroxy, —O—(CH2)y-lower alkoxy, —O(CH2)yC(O)N(lower alkyl)2, —C(O)-lower alkyl, —O—(CH2)x-aryl, —O—(CH2)x-cycloalkyl, —O—(CH2)x-heterocycloalkyl, —C(O)O-lower alkyl, —C(O)—NH-lower alkyl, —C(O)—N(lower alkyl)2, 2-oxy-5-aza-bicyclo[2.2.1]hept-5-yl or 3-oxa-8-aza-bicyclo[3.2.1]oct-8-yl; R, R′, R″ and R′″ are each independently hydrogen or lower allyl; or R′ and R′″ in group e) together with —(CH2)4- form a six membered ring; and wherein all aryl-, cycloalkyl-, cyclic amide, heterocycloalkyl- or 5 or 6 membered heteroaryl groups as defined for R1, R1′, R1″ and R3 to R10 are unsubstituted or substituted by one or more substituents selected from the group consisting of hydroxy, ═O, halogen, lower alkyl, phenyl, lower alkyl substituted by halogen and lower alkoxy; n, m, o, p, q, r, s and t are each independently 1 or 2; x is 0, 1 or 2; and y is 1 or 2; or a pharmaceutically acceptable salt thereof.

In certain embodiments, the pharmaceutical composition further comprises a pharmaceutically acceptable carrier.

In certain embodiments, the subject is a subject in need thereof.

In certain embodiments, the GlyT1 inhibitor, or pharmaceutically acceptable salt thereof, or prodrug of the GlyT1 inhibitor or its pharmaceutically acceptable salt, is administered in a therapeutically effective amount.

### BRIEF DESCRIPTION OF THE DRAWINGS

FIG. 1 shows the expression of GlyT1 in various liver-derived cell lines as compared to expression in an erythropoietic cell line, K562.

FIG. 2 shows the expression of GlyT1 and other components of the heme synthesis pathway (e.g., ALAS1, ALAS2, ALAD, FECH, CPOX, HMBS, UROD, UROS, and GlyT2) in 26 liver cancer cell lines.

FIG. 3 shows phenobarbital induces overexpression of ALAS1 (FIG. 3A) and GlyT1 (FIG. 3B) in HepG2 Cells. In this figure, “*” indicates a p value of <0.05 and “****” indicates a p value of <0.0001

FIG. 4 shows that bitopertin inhibited glycine uptake in HepG2 cells overexpressing GlyT1. FIG. 4A represents the HepG2 pLenti6.3-GlyT1 (untagged) and FIG. 4B represents HepG2 pLenti6.3-GlyT1-HA-Flag.

FIG. 5 shows the reduction of toxic metabolites 5-ALA (FIG. 5A) and PBG (FIG. 5B) by the treatment of the GlyT1 inhibitor bitopertin in HepG2 cells transduced with shHMBS, GlyT1, ALAS1.

FIG. 6 shows reduction of toxic metabolites 5-ALA (FIG. 6A) and PBG (FIG. 6B) by the treatment of a GlyT1 inhibitor, bitopertin in HepG2 cells transduced with shHMBS, GlyT1, ALAS1 in the presence of different concentrations of glycine in the medium. In this figure, “*” indicates a p value of <0.05, “**” indicates a p value of <0.01, and “***” indicates a p value of <0.001.

### DETAILED DESCRIPTION OF THE APPLICATION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

The term “acyl” is art-recognized and refers to a group represented by the general formula hydrocarbylC(O)—, preferably alkylC(O)—.

As used herein, the term “acylamino” means an amino group substituted by an acyl group (e.g., —O—C(═O)—H or —O—C(═O)-alkyl). An example of an acylamino is —NHC(═O)H or —NHC(═O)CH₃. The term “lower acylamino” refers to an amino group substituted by a lower acyl group (e.g., —O—C(═O)—H or —O—C(═O)—C₁₋₆alkyl). An example of a lower acylamino is —NHC(═O)H or —NHC(═O)CH₃.

The term “acyloxy” is art-recognized and refers to a group represented by the general formula hydrocarbylC(O)O—, preferably alkylC(O)O—.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

The terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylamino” means an amino group substituted by an alkyl group having from 1 to 6 carbon atoms. An example of an alkylamino is —NHCH₂CH₃.

As used herein, the term “alkylene” or “alkylenyl” means a divalent alkyl linking group. An example of an alkylene (or alkylenyl) is methylene or methylenyl (—CH₂—).

As used herein, the term “alkylthio” means an —S-alkyl group having from 1 to 6 carbon atoms. An example of an alkylthio group is —SCH₂CH₃.

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

The term “amide”, as used herein, refers to a group

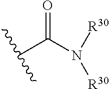

wherein each R³⁰ independently represent a hydrogen or hydrocarbyl group, or two R³⁰ are taken together with the N atom to which they are attached complete a heterocycle having from 4 to 8 atoms in the ring structure.

As used herein, the term “amidino” means —C(═NH)NH₂.

The terms “amine” and “amino” are art-recognized and refer to both unsubstituted and substituted amines and salts thereof, e.g., a moiety that can be represented by

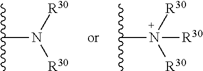

wherein each R³⁰ independently represents a hydrogen or a hydrocarbyl group, or two R³⁰ are taken together with the N atom to which they are attached complete a heterocycle having from 4 to 8 atoms in the ring structure.

As used herein, the term “aminoalkoxy” means an alkoxy group substituted by an amino group. An example of an aminoalkoxy is —OCH₂CH₂NH₂.

As used herein, the term “aminoalkyl” means an alkyl group substituted by an amino group. An example of an aminoalkyl is —CH₂CH₂NH₂.

As used herein, the term “aminosulfonyl” means —S(═O)₂NH₂.

As used herein, the term “aminoalkylthio” means an alkylthio group substituted by an amino group. An example of an aminoalkylthio is —SCH₂CH₂NH₂.

As used herein, the term “amphiphilic” means a three-dimensional structure having discrete hydrophobic and hydrophilic regions. An amphiphilic compound suitably has the presence of both hydrophobic and hydrophilic elements.

As used herein, the term “animal” includes, but is not limited to, humans and non-human vertebrates such as wild, domestic, and farm animals.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. Examples of aryl groups include, but are not limited to:

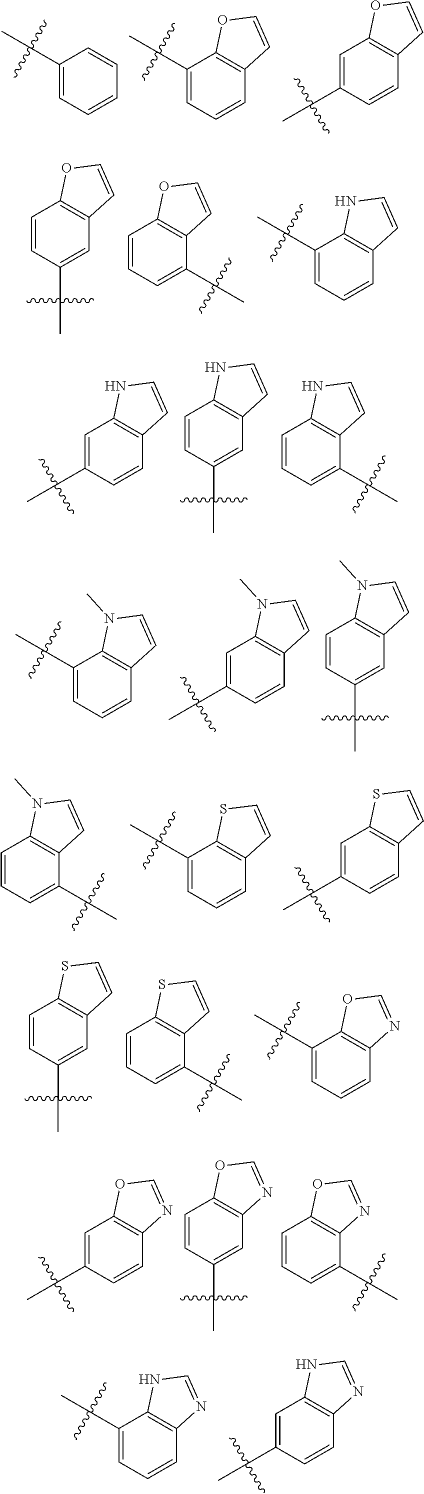

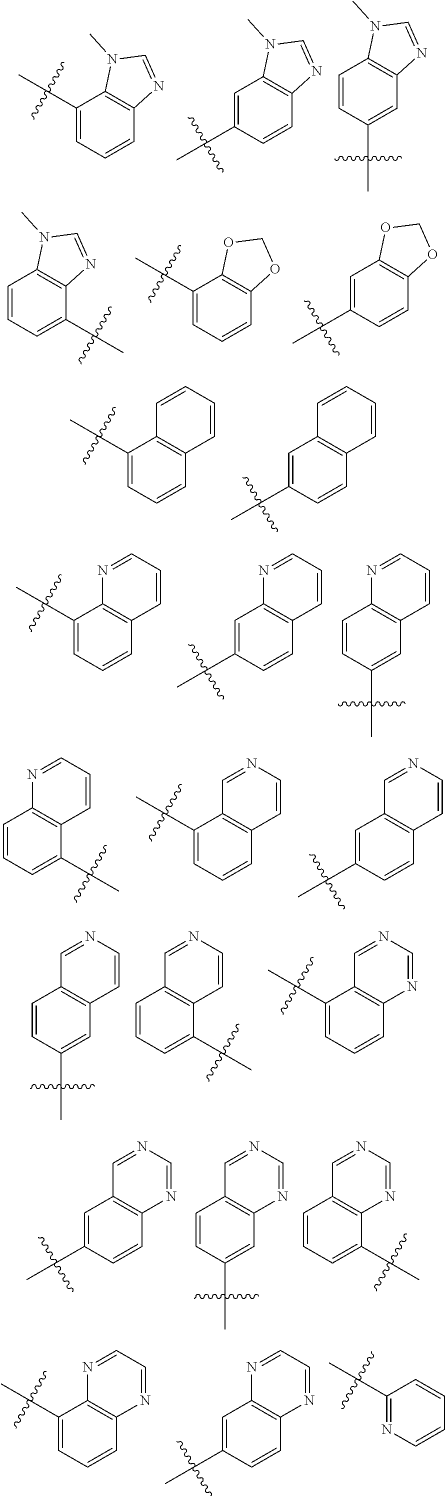

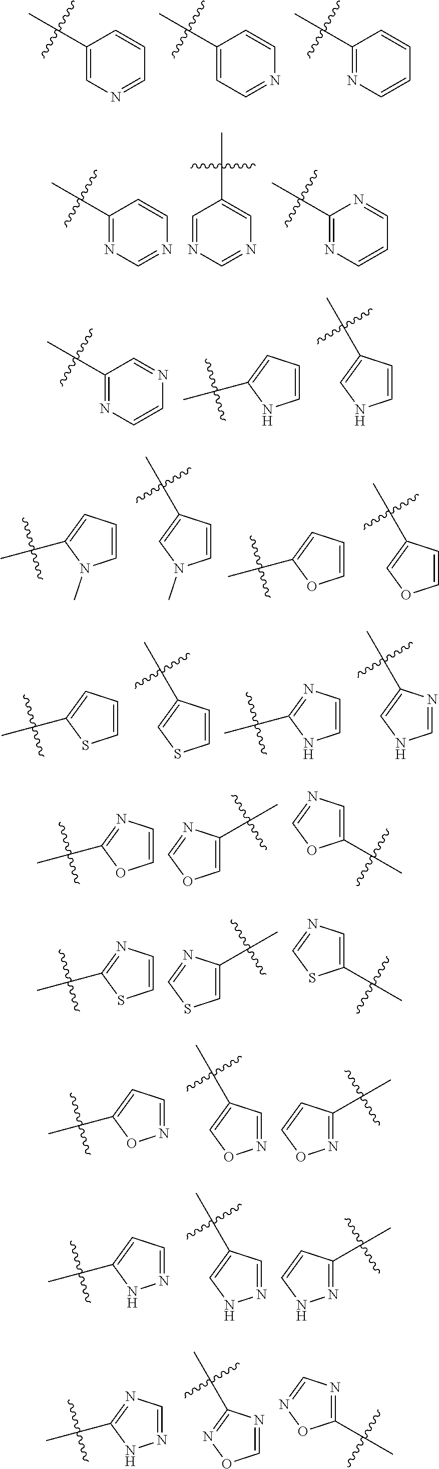

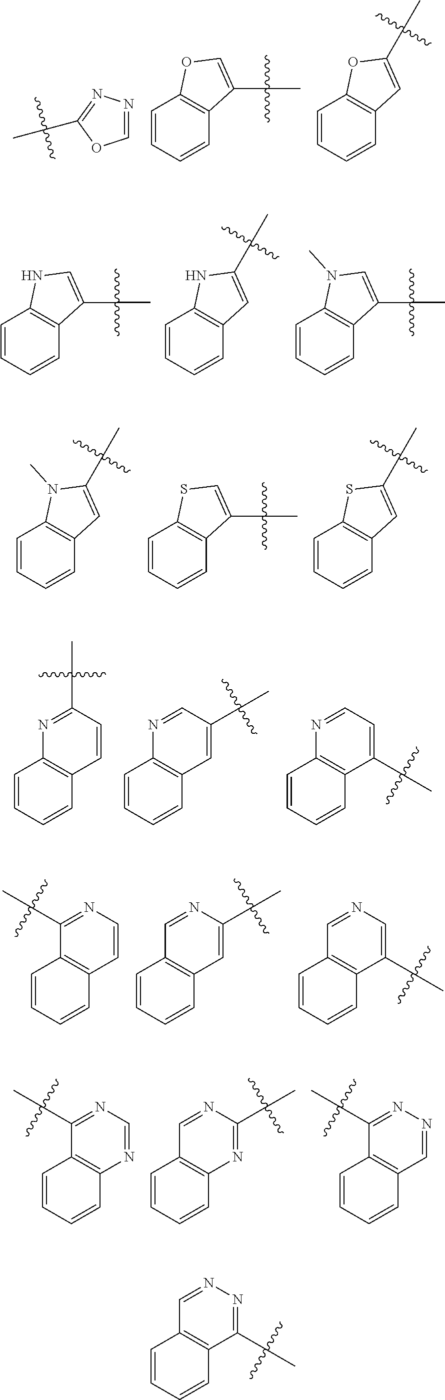

As used herein, the term “arylalkyl” means a C₁₋₆alkyl substituted by aryl.

As used herein, the term “arylamino” means an amino group substituted by an aryl group. An example of an arylamino is —NH(phenyl).

As used herein, the term “arylene” means an aryl linking group, i.e., an aryl group that links one group to another group in a molecule.

The term “carbamate” is art-recognized and refers to a group

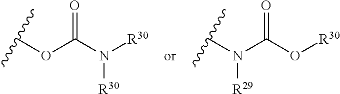

wherein R²⁹ and R³⁰ independently represent hydrogen or a hydrocarbyl group, such as an alkyl group, or R²⁹ and R³⁰ taken together with the intervening atom(s) complete a heterocycle having from 4 to 8 atoms in the ring structure.

As used herein, the term “carbamoyl” means —C(═O)—NH₂.

As used herein, the term “carbocycle” means a 5- or 6-membered, saturated or unsaturated cyclic ring, optionally containing O, S, or N atoms as part of the ring. Examples of carbocycles include, but are not limited to, cyclopentyl, cyclohexyl, cyclopenta-1,3-diene, phenyl, and any of the heterocycles recited above.

The term “carbocyclylalkyl”, as used herein, refers to an alkyl group substituted with a carbocycle group.

The term “carbonate” is art-recognized and refers to a group —OCO₂—R³⁰, wherein R³⁰ represents a hydrocarbyl group.

The term “carboxy”, as used herein, refers to a group represented by the formula —CO₂H.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a GlyT1 transporter inhibitor with a GlyT1 transporter with an individual or patient or cell includes the administration of the compound to an individual or patient, such as a human, as well as, for example, introducing a compound into a sample containing a cellular or purified preparation containing the GlyT1 transporter.

As used herein, the term “cyano” means —CN.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “cycloalkylalkyl” means a C₁₋₆alkyl substituted by cycloalkyl.

As used herein, the term “dialkylamino” means an amino group substituted by two alkyl groups, each having from 1 to 6 carbon atoms.

As used herein, the term “diazamino” means —N(NH₂)₂.

The term “ester”, as used herein, refers to a group —C(O)OR³⁰ wherein R³⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

As used herein, the term “facially amphiphilic” or “facial amphiphilicity” means compounds with polar (hydrophilic) and nonpolar (hydrophobic) side chains that adopt conformation(s) leading to segregation of polar and nonpolar side chains to opposite faces or separate regions of the structure or molecule.

As used herein, the term “glycine transporter” or “GlyT” refers to membrane protein that facilitates the transport of glycine across the plasma membrane of a cell. Non-limiting examples of glycine transports include glycine transporter 1 (GlyT1) and glycine transporter 2 (GlyT2).

As used herein, the term “GlyT1” or “GlyT1 transporter” means sodium- and chloride-dependent glycine transporter 1, also known as glycine transporter 1, is a protein that in humans is encoded by the SLC6A9 gene (Kim K M, Kingsmore S F, Han H, Yang-Feng T L, Godinot N, Seldin M F, Caron M G, Giros B (June 1994). “Cloning of the human glycine transporter type 1: molecular and pharmacological characterization of novel isoform variants and chromosomal localization of the gene in the human and mouse genomes”. Mol Pharmacol. 45 (4): 608-17; Jones E M, Fernald A, Bell G I, Le Beau M M (November 1995). “Assignment of SLC6A9 to human chromosome band 1p33 by in situ hybridization”. Cytogenet Cell Genet. 71 (3): 211), which is hereby incorporated by reference in its entirety.

As used herein, the term “GlyT2” or “GlyT2 transporter” means sodium- and chloride-dependent glycine transporter 2, also known as glycine transporter 2, is a protein that in humans is encoded by the SLC6A5 gene (Morrow J A, Collie I T, Dunbar D R, Walker G B, Shahid M, Hill D R (November 1998). “Molecular cloning and functional expression of the human glycine transporter GlyT2 and chromosomal localisation of the gene in the human genome”. FEBS Lett. 439 (3): 334-40), which is hereby incorporated by reference in its entirety.

As used herein, the term “GlyT1 inhibitor” means a compound that inhibits or blocks the activity of GlyT1 transporter including compounds inhibiting the activity of any isoform of GlyT1. Non-limiting examples of GlyT1 inhibitors are provided herein. In some embodiments, the GlyT1 inhibitor is a specific GlyT1 inhibitor, which means that the inhibitor has an inhibitor activity that is greater for GlyT1 as compared to GlyT2. In some embodiments, the inhibitor inhibits GlyT1 as compared to GlyT2 with at least, or about, 10%, 20%, 30%, 40%, 50%, 60%, 70%, 80%, 90%, 95%, 96%, 97%, 98%, 99% selectivity. In some embodiments, the GlyT1 inhibitor inhibits GlyT1 but does not inhibit or significantly inhibit the activity of GlyT2. A GlyT1 inhibitor that does not significantly inhibit the activity of GlyT2 if it inhibits the activity of GlyT2 less than 5%, 4%, 3%, 2%, or 1%. The selectivity of GlyT1 inhibitor is determined based on the known assays in the art such as the assays described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety.

As used herein, the term “GlyT2 inhibitor” means a compound that inhibits or blocks the activity of GlyT2 transporter including compounds inhibiting the activity of any isoform of GlyT2. In some embodiments, the GlyT2 inhibitor is a non-specific inhibitor, which means that it can also inhibit or block the activity of GlyT1. In some embodiments, the GlyT2 inhibitor is a specific GlyT2 inhibitor, which means that the inhibitor has an inhibitor activity that is greater for GlyT2 as compared to GlyT1. In some embodiments, the inhibitor inhibits GlyT2 as compared to GlyT1 with at least, or about, 10%, 20%, 30%, 40%, 50%, 60%, 70%, 80%, 90%, 95%, 96%, 97%, 98%, 99% selectivity. In some embodiments, the GlyT2 inhibitor inhibits GlyT2 activity but does not inhibit or significantly inhibit the activity of GlyT1. A GlyT2 inhibitor that does not significantly inhibit the activity of GlyT1 if it inhibits the activity of GlyT1 less than 5%, 4%, 3%, 2%, or 1%. The selectivity of GlyT2 inhibitor is determined based on the known assays in the art such as the assays based described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety.

As used herein, the term “guanidino” means —NH(═NH)NH₂.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆alkyl group having one or more halogen substituents. Examples of haloalkyl groups include, but are not limited to, CF₃, C₂F₅, CH₂F, CHF₂, CCl₃, CHCl₂, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which is, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylalkyl” means a C₁₋₆alkyl group substituted by a heteroaryl group.

As used herein, the term “heteroarylamino” means an amino group substituted by a heteroaryl group. An example of a heteroarylamino is —NH-(2-pyridyl).

As used herein, the term “heteroarylene” means a heteroaryl linking group, i.e., a heteroaryl group that links one group to another group in a molecule.

The term “heteroatom” as used herein means an atom of any element other than carbon or hydrogen. Exemplary heteroatoms are nitrogen, oxygen, and sulfur.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “heterocycloalkylalkyl” refers to a C₁₋₆alkyl substituted by heterocycloalkyl.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or transporter activity means reducing by any measurable amount the activity of an enzyme or transporter, such as the GlyT1 transporter.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “in situ gellable” means embracing not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

As used herein, the phrase “integer from X to Y” means any integer that includes the endpoints. For example, the phrase “integer from X to Y” means 1, 2, 3, 4, or 5.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “N-alkyl” refers to a alkyl chain that is substituted with an amine group. Non-limiting examples, include, but are not limited to

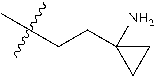 [SMILES: *CCC1(N)CC1]

and the like. The alkyl chain can be linear, branched, cyclic, or any combination thereof. In some embodiments, the alkyl comprises 1-10, 1-9, 1-8, 1-7, 1-6, 1-5, 1-4, 1-3, or 1-2 carbons.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

For a compound described herein that contains a basic group, such as an amine, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

For a compound described herein that contains an acidic group, such as a carboxylic acid group, base addition salts can be prepared by any suitable method available in the art, for example, treatment of such compound with a sufficient amount of the desired the desired base, either neat or in a suitable inert solvent. Examples of pharmaceutically acceptable base addition salts include, but are not limited to, lithium, sodium, potassium, calcium, ammonium, zinc, or magnesium salt, or other metal salts; organic amino salts, such as, alkyl, dialkyl, trialkyl, or tetra-alkyl ammonium salts.

Other examples of pharmaceutically acceptable salts include, but are not limited to, camsylate, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present application.

As used herein, the term “phenyl” means —C₆H₅. A phenyl group can be unsubstituted or substituted with one, two, or three suitable substituents.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “prodrug” means a derivative of a known direct acting drug, which derivative has enhanced delivery characteristics and therapeutic value as compared to the drug, and is transformed into the active drug by an enzymatic or chemical process. A common method for making a prodrug is to include one or more selected moieties which are hydrolyzed under physiologic conditions to yield the desired molecule. In certain embodiments, the prodrug is converted by an enzymatic activity of the host animal. For example, a prodrug with a nitro group on an aromatic ring could be reduced by reductase to generate the desired amino group of the corresponding active compound in vivo. In another example, functional groups such as a hydroxyl, carbonate, or carboxylic acid in the parent compound are presented as an ester, which could be cleaved by esterases. Additionally, amine groups in the parent compounds are presented in, but not limited to, carbamate, N-alkylated or N-acylated forms (Simplicio et al, “Prodrugs for Amines,” Molecules, (2008), 13:519-547). In certain embodiments, some or all of the compounds of described herein in a formulation represented above can be replaced with the corresponding suitable prodrug.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the term “semicarbazone” means ═NNHC(═O)NH₂.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

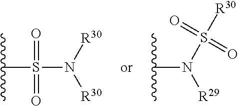

wherein R²⁹ and R³⁰ independently represents hydrogen or hydrocarbyl, such as alkyl, or R²⁹ and R³⁰ taken together with the intervening atom(s) complete a heterocycle having from 4 to 8 atoms in the ring structure.

The term “sulfoxide” is art-recognized and refers to the group —S(O)—R³⁰, wherein R³⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R³⁰, wherein R³⁰ represents a hydrocarbyl.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

The term “thioalkyl”, as used herein, refers to an alkyl group substituted with a thiol group.

The term “thioester”, as used herein, refers to a group —C(O)SR³⁰ or —SC(O)R³⁰ wherein R³⁰ represents a hydrocarbyl.

The term “thioether”, as used herein, is equivalent to an ether, wherein the oxygen is replaced with a sulfur.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of a hepatic porphyria” means an activity that alleviates or ameliorates any of the primary phenomena or secondary symptoms associated with the hepatic porphyria or other conditions described herein.

The term “urea” is art-recognized and may be represented by the general formula

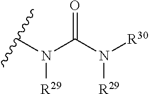

wherein R²⁹ and R³⁰ independently represent hydrogen or a hydrocarbyl, such as alkyl, or either occurrence of R²⁹ taken together with R³⁰ and the intervening atom(s) complete a heterocycle having from 4 to 8 atoms in the ring structure.

At various places in the present specification, substituents of compounds may be disclosed in groups or in ranges. It is specifically intended that embodiments include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

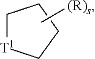

then it is understood that substituent R can occur s number of times on the ring, and R can be a different moiety at each occurrence. In the above example, where the variable T¹ is defined to include hydrogens, such as when T¹ is CH₂, NH, etc., any H can be replaced with a substituent.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompasses the use, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are provided herein. Cis and trans geometric isomers of the compounds are also included within the present embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

In some embodiments, the composition comprises a compound, or a pharmaceutically acceptable salt, solvate or prodrug thereof, that is at least 90%, at least 95%, at least 98%, or at least 99%, or 100% enantiomeric pure, which means that the ratio of one enantiomer to the other in the composition is at least 90:1 at least 95:1, at least 98:1, or at least 99:1, or is completely in the form of one enantiomer over the other. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Glycine transporter inhibitors, such as GlyT1 inhibitors, including their pharmaceutically acceptable salts (e.g., the GlyT1 inhibitors as disclosed herein) can also exist as hydrates and solvates, as well as anhydrous and non-solvated forms. A “hydrate” is a compound that exists in a composition with water molecules. The composition can include water in stoichiometric quantities, such as a monohydrate or a dihydrate, or can include water in random amounts. A “solvate” is a similar composition except that a solvent other that water, such as with methanol, ethanol, dimethylformamide, diethyl ether and the like replaces the water. For example, methanol or ethanol can form an “alcoholate,” which can again be stoichiometric or non-stoichiometric. Mixtures of such solvates or hydrates can also be prepared. The source of such solvate or hydrate can be from the solvent of crystallization, inherent in the solvent of preparation or crystallization, or adventitious to such solvent.

The compounds of the application, including their pharmaceutically acceptable salts and prodrugs, can exist as various polymorphs, pseudo-polymorphs, or in amorphous state. The term “polymorph”, as used herein, refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus crystalline polymorphic forms are important aspects in the development of suitable dosage forms in pharmaceutical industry.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti-arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Embodiments of various compounds and salts thereof are provided. Where a variable is not specifically recited, the variable can be any option described herein, except as otherwise noted or dictated by context.

In some embodiments, the compound is as described in the appended exemplary, non-limiting claims, or a pharmaceutically acceptable salt, solvate or prodrug thereof.

In some embodiments of the methods and uses disclosed herein, the GlyT1 inhibitor is a compound of Formula I,

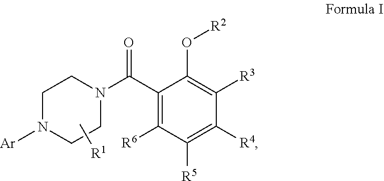

wherein:
Ar is unsubstituted or substituted aryl or 6-membered heteroaryl containing one, two or three nitrogen atoms, wherein the substituted aryl and the substituted heteroaryl groups are substituted by one or more substituents selected from the group consisting of hydroxy, halogen, NO₂, CN, (C₁-C₆)-alkyl, (C₁-C₆)-alkyl substituted by halogen, (C₁-C₆)-alkyl substituted by hydroxy, (CH₂)ₙ—(C₁-C₆)-alkoxy, (C₁-C₆)-alkoxy substituted by halogen, NR⁷R⁸, C(O)R⁹, SO₂R¹⁰, and —C(CH₃)=NOR⁷, or are substituted by a 5-membered aromatic heterocycle containing 1-4 heteroatoms selected from N and O, which is optionally substituted by (C₁-C₆)-alkyl;
R¹ is hydrogen or (C₁-C₆)-alkyl;
R² is hydrogen, (C₁-C₆)-alkyl, (C₂-C₆)-alkenyl, (C₁-C₆)-alkyl substituted by halogen, (C₁-C₆)-alkyl substituted by hydroxy, (CH2)n-(C₃-C₇)-cycloalkyl optionally substituted by (C₁-C₆)-alkoxy or by halogen, CH(CH₃)—(C₃-C₇)-cycloalkyl, (CH₂)ₙ₊₁—C(O)—R⁹, (CH₂)ₙ₊₁—CN, bicyclo[2.2.1]heptyl, (CH₂)ₙ₊₁—O—(C₁-C₆)-alkyl, (CH₂)ₙ-heterocycloalkyl, (CH₂)ₙ-aryl or (CH₂)ₙ-5 or 6-membered heteroaryl containing one, two or three heteroatoms selected from the group consisting of oxygen, sulphur or nitrogen wherein aryl, heterocycloalkyl and heteroaryl are unsubstituted or substituted by one or more substituents selected from the group consisting of hydroxy, halogen, (C₁-C₆)-alkyl and (C₁-C₆)-alkoxy;
R³, R⁴ and R⁶ are each independently hydrogen, hydroxy, halogen, (C₁-C₆)-alkyl, (C₁-C₆)-alkoxy or O—(C₃-C₆)-cycloalkyl;
R⁵ is NO₂, CN, C(O)R⁹ or SO₂R¹⁰;
R⁷ and R⁸ are each independently hydrogen or (C₁-C₆)-alkyl;
R⁹ is hydrogen, (C₁-C₆)-alkyl, (C₁-C₆)-alkoxy or NR⁷R⁸;
R¹⁰ is (C₁-C₆)-alkyl optionally substituted by halogen, (CH₂)ₙ—(C₃-C₆)-cycloalkyl, (CH₂)ₙ—(C₃-C₆)-alkoxy, (CH₂)ₙ-heterocycloalkyl or NR⁷R⁸;
n is 0, 1, or 2;
or a pharmaceutically acceptable salt thereof, or a prodrug of the compound or its pharmaceutically acceptable salt.

In some embodiments of the methods and uses disclosed herein, the GlyT1 inhibitor is a compound having a formula of

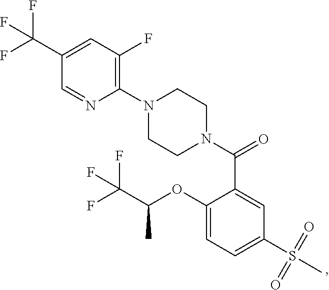 [SMILES: C[C@H](Oc1ccc(S(C)(=O)=O)cc1C(=O)N1CCN(c2ncc(C(F)(F)F)cc2F)CC1)C(F)(F)F]

bitopertin, or a pharmaceutically acceptable salt thereof, or a prodrug of the compound or its pharmaceutically acceptable salt.

In some embodiments of the methods and uses disclosed herein, the GlyT1 inhibitor is a compound of Formula II,

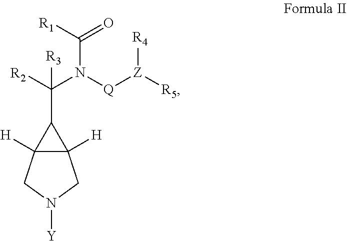

wherein:
R₁ represents a heteroaryl selected from the group consisting of: imidazolyl, thiazolyl, pyridyl, oxazolyl, pyrazolyl, triazolyl, oxadiazolyl, quinolinyl, isoxazolyl, pyrroloimidazoyl, and thiadiazole, wherein said heteroaryl is optionally substituted by one or more substituents selected from —OH, —NR₇R₈, halogen, (C₁-C₈)alkyl, (C₃-C₁₀)cycloalkyl, (C₁-C₈)alkoxy, (C₁-C₁₂)alkoxyalkyl, (C₁-C₈)hydroxyalkyl, (C₆-C₁₄)aryl and benzyl;
R₂, R₃ and A independently represent H or (C₁-C₈)alkoxy, wherein said alkyl is optionally substituted by one or more —OH, (C₁-C₈)alkoxy, —NR₇R₈ or halogen;
Q represents —(CH₂)ₙ—, where n=1, 2, 3 or 4 or —(CH₂)ₘ—O—, where m=2, 3 or 4;
Z represents (C₆-C₁₄)aryl, (C₁-C₈)alkyl or (C₃-C₈)cycloalkyl;
R₄ and R₅ each independently represent H, halogen, (C₁-C₈)alkyl, (C₆-C₁₄)aryl, (C₆-C₁₄)aryloxy, (C₁-C₈)alkoxy, (3-10 membered)heterocycloalkyl or (C₃-C₈)cycloalkoxy; wherein R₄ and R₅ are optionally substituted by one or more —OH, (C₁-C₈)alkoxy, —NR₇R₈ or halogen;
Y represents —R₆, —(CH₂)o-R₆, —C(R₆)₃ or —CH(R₆)₂, wherein 0=1, 2 or 3;
R₆ represents H, (C₆-C₁₄)aryl, (C₁₋₁₀)alkyl, (C₃-C₁₀)cycloalkyl, (C₅-C₁₈)bicycloalkyl, (C₅-C₁₈)tricycloalkyl, (3-10 membered)heterocycloalkyl, (5-10 membered)heteroaryl, —C(═O)NR₇R₈, or —C(═O)OR₇, wherein said R₆ groups can optionally be substituted with one or more X groups;
wherein X═—OH, (C₁-C₈)alkoxy, —NR₁₁R₁₂, —SO₂R₁₀, —C(═O)R₁₀, halogen, cyano, (C₁-C₈)alkyl, (C₁-C₁₀)alkoxyalkyl, (5-10 membered)heteroaryl, (C₆-C₁₄)aryl, (C₆-C₁₄)aryloxy, benzyl, or (C₁-C₈)hydroxyalkyl;
wherein R₇ and R₈ independently represent H, (C₁-C₈)alkyl, (C₃-C₈)cycloalkyl, (5-10 membered)heterocycloalkyl, (C₁-C₈)hydroxyalky, (5-10 membered)heteroaryl or (C₁-C₁₀)alkoxyalkyl; wherein R₇ and R₈ may optionally be substituted by one or more X groups;
or R₇ and R₈ together with the nitrogen in which they may be attached may form a (3-10 membered)heterocycloalkyl group optionally substituted by one or more X groups;
wherein R₁₀ represents (C₁-C₈)alkyl, (C₃-C₈)cycloalkyl, (3-10 membered)heterocycloalkyl, (C₁-C₈)hydroxyalky, (5-10 membered)heteroaryl or (C₁-C₁₀)alkoxyalkyl;
wherein R₁₁ and R₁₂ independently represent H, (C₁-C₈)alkyl, (C₃-C₈)cycloalkyl, (5-10 membered)heterocycloalkyl, (C₁-C₈)hydroxyalky, (5-10 membered)heteroaryl or (C₁-C₁₀)alkoxyalkyl; or a pharmaceutically acceptable salt thereof, or a prodrug of the compound or its pharmaceutically acceptable salt.

In some embodiments of the methods and uses disclosed herein, the GlyT1 inhibitor is a compound having a formula of

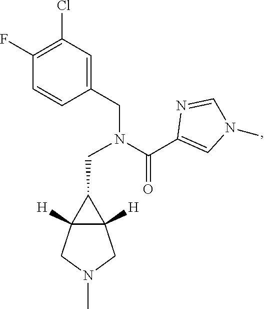

or a pharmaceutically acceptable salt thereof, or a prodrug of the compound or its pharmaceutically acceptable salt.

In some embodiments of the methods and uses disclosed herein, the GlyT1 inhibitor is a compound having a formula of

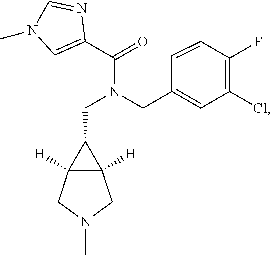 [SMILES: CN1C[C@@H]2[C@H](C1)[C@H]2CN(Cc1ccc(F)c(Cl)c1)C(=O)c1cn(C)cn1]

PF-3463275, or a pharmaceutically acceptable salt thereof, or a prodrug of the compound or its pharmaceutically acceptable salt.

In some embodiments of the methods and uses disclosed herein, the GlyT1 inhibitor is a compound of Formula III,

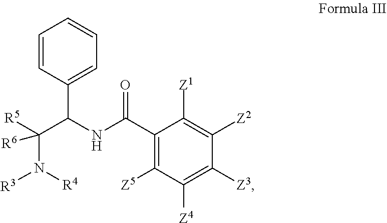

wherein:
Z¹ is selected from the group consisting of C₁₋₄alkyl, C₃₋₆CycloalkVI, C₁₋₄alkoxy, C₁₋₄alkylthio, haloC₁₋₄alkyl, phenyl, haloC₁₋₄alkoxy, halophenyl, C₁₋₄alkylsulfoxy, C₁-4alkylsulfonyl, bromo and chloro;
Z² is selected from the group consisting of hydrogen, halogen, cyano, C₁₋₄alkyl, phenyl, haloC₁₋₄alkyl, haloC₁₋₄alkoxy, halophenyl, C₁₋₄alkoxyC₁₋₄alkyl and C₃₋₆cycloalkyl;
Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl;
Z⁴ is selected from the group consisting of hydrogen, halogen, C₁₋₃alkyl, haloC₁₋₄ alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl;
Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl;
whereby if more than one of Z¹ to Z⁵ is methoxy, then only Z¹ and Z⁵ are methoxy R³ and R⁴ are independently selected from hydrogen and C₁₋₄alkyl, optionally substituted with one or more groups Y; or R³ and R4 together with the nitrogen atom to which they are attached form a saturated or partially unsaturated A-, 5-6- or 7-membered carbocyclic ring optionally substituted with a group Y′;
Y is selected from the group consisting of C₁₋₄alkoxy, hydroxy, haloC₁₋₄alkoxy and C₃₋₆cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH2- or —CH2-CH2- bridge between two atoms on the A-, 5-, 6- or 7-membered carbocyclic ring;
R⁵ and R⁶ are independently C₁₋₄alkyl, optionally substituted with one or more groups X; or R⁵ and R⁶ together with the carbon atom to which they are attached form a saturated 5- or 6-membered ring carbocyclic optionally substituted with one or more groups X′, in the case of R⁵ and R6 together with the carbon atom to which they are attached forming a 5-membered saturated carbocyclic ring, that ring may optionally further comprising an additional heteroatom group selected from O, N and S(O)m; where m=0, 1 or 2.
X is selected from the group consisting of halogen, hydroxy, C₁₋₄alkoxy, haloC₁₋₄alkyl, haloC₁₋₄alkoxy and C₅₋₁₀aryl; and
X′ is selected from the group consisting of halogen, hydroxy, C₁₋₄alkyl, C₁₋₄alkoxy, haloC₁₋₄alkyl, haloC₁₋₄alkoxy and C₅₋₁₀aryl;
whereby R³, R⁴, R⁵ and R⁶ are not all simultaneously unsubstituted methyl;
with the provisos that when simultaneously Z¹ is propyloxy, Z³ is chloro, Z²=Z⁴=Z⁵=H, and R⁵ and R⁶ are both methyl, then R³ and R⁴ together with the nitrogen atom to which they are attached do not form a 2-methylpyrrolidine group; when simultaneously Z¹ is methyl, Z³ is methoxy, Z²=Z4=Z5=H, and R⁵ and R⁶ are both methyl, then R³ and R⁴ together with the nitrogen atom to which they are attached do not form a pyrrolidine group, or a pharmaceutically acceptable salt thereof, or a prodrug of the compound or its pharmaceutically acceptable salt.

In some embodiments of the methods and uses disclosed herein, the GlyT1 inhibitor is a compound having a formula of

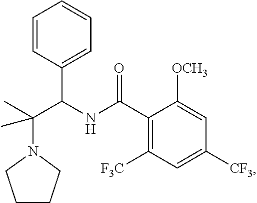 [SMILES: COc1cc(C(F)(F)F)cc(C(F)(F)F)c1C(=O)NC(c1ccccc1)C(C)(C)N1CCCC1]

or a pharmaceutically acceptable salt thereof, or a prodrug of the compound or its pharmaceutically acceptable salt.

In some embodiments of the methods and uses disclosed herein, the GlyT1 inhibitor is a compound of Formula IV,

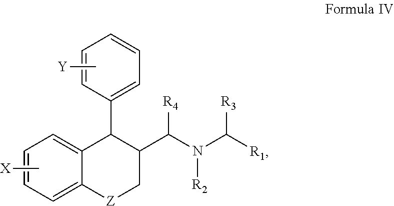

wherein:
Z is (CH₂)ₙ, O, S, SO, SO₂ or N—R₅;
n is 0, 1 or 2;
X represents 1-3 substituents independently selected from hydrogen, halogen, (C₁₋₆)alkyioxy, (C₃₋₆)cycloalkyloxy, (C₆₋₁₂)aryloxy, (C₆₋₁₂)aryl, thienyl, SR6, SOR6,
SO₂R₆, NR₆R₆, NHR₆, NH₂, NHCOR₆, NSO₂R₆, CN, COOR₆ and (C₁₋₄)alkyl, optionally substituted with halogen, (C₆₋₁₂)aryl, (C₁₋₆)alkyloxy or (C₆₋₁₂)aryloxy; or 2 substituents at adjacent positions together represent a fused (C₅₋₆)aryl group, a fused (C₅₋₆)cycloalkyl ring or O—(CH₂)ₘ—O; m is 1 or 2;
Y represents 1-3 substituents independently selected from hydrogen, halogen, (C₁₋₄)alkyloxy, SR₆, NR₆R₆ and (C₁₋₄)alkyl, optionally substituted with halogen;
R₁ is COOR₇ or CONR₈R₉;
R₂ and R₆ are (C₁₋₄)alkyl;
R₃, R₄ are R₅ are independently hydrogen or (C₁₋₄)alkyl;
R₇, R₈ and R₉ are independently hydrogen, (C₁₋₄)alkyl, (C₆₋₁₂)aryl or arylalkyl, or a pharmaceutically acceptable salt thereof, or a prodrug of the compound or its pharmaceutically acceptable salt.

In some embodiments of the methods and uses disclosed herein, the GlyT1 inhibitor is a compound having a formula of

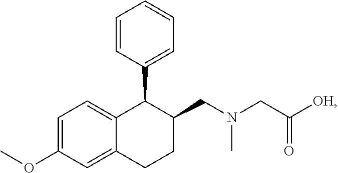 [SMILES: COc1ccc2c(c1)CC[C@H](CN(C)CC(=O)O)[C@@H]2c1ccccc1]

ORG-25935, or a pharmaceutically acceptable salt thereof, or a prodrug of the compound or its pharmaceutically acceptable salt.

In some embodiments of the methods and uses disclosed herein, the GlyT1 inhibitor is a compound of Formula V,

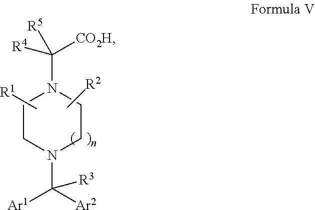

wherein:
n is an integer from 1 to 3;
R¹ and R² are independently selected from hydrogen, alkyl, haloalkyl, alkoxy, haloalkoxy, aryl, heteroaryl, cycloalkyl, or heterocyclyl wherein the aforementioned rings are optionally substituted with R^{a}, R^{b}, or R^{c} independently selected from alkyl, halo, haloalkyl, alkoxy, haloalkoxy, hydroxy, cyano, monosubstituted amino, or disubstituted amino; or
R¹ and R², when attached to the same carbon atom, can combine to form cycloalkyl or monocyclic saturated heterocyclyl to give a spiro ring wherein the cycloalkyl or monocyclic saturated heterocyclyl can be optionally substituted with R^{d}, R^{c}, or R^{f} independently selected from alkyl, alkoxy, fluoro, fluoroalkyl, fluoroalkoxy, hydroxy, monosubstituted amino, or disubstituted amino; or
R¹ and R², when attached to carbon atoms 2 and 5 or 3 and 6 positions of the piperazine ring, can combine to form —C₁-C₃-alkylene chain wherein one of the carbon atoms in the alkylene chain is optionally replaced by a —NR—, —O—, —S(O)n- (where R is hydrogen or alkyl and n is 0-2) and further wherein one or two hydrogen atoms in the alkylene chain can be optionally substituted with one or two alkyl;
R³, R⁴ and R⁵ are independently hydrogen, alkyl, fluoro, or fluoroalkyl; and Ar¹ and Ar² are independently aryl, heteroaryl, cycloalkyl, or heterocyclyl where each of the aforementioned ring is optionally substituted with R^{g}, R^{h} or Rⁱ where R^{g} is alkyl, —C═C—R⁶ (where R⁶ is aryl or heteroaryl), halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{h} and Rⁱ are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl., heteroaryl, cycloalkyl, or heterocyclyl where the aromatic or alicyclic ring-in R^{g}, R^{h} and Rⁱ is optionally substituted with R^{j}, R^{k}, or R^{l} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or a pharmaceutically acceptable salt thereof provided that: the compound of Formula V is not 2-(4-benzhydrylpiperazin-1-yl)acetic acid, 2-(4-((4-chlorophenyl)(phenyl)methyl)piperazin-1-yl)acetic acid, 2-((2R,5S)-4-((R)-(4-(1H-tetrazol-5-yl)phenyl)(3-hydroxyphenyl)methyl)-2,5-dimethylpiperazin-1-yl)acetic acid, or 2-((2R,5S)-4-((R)-(4-cyanophenyl)(3-hydroxyphenyl)methyl)-2,5-dimethylpiperazin-1-yl)acetic acid, or a pharmaceutically acceptable salt thereof, or a prodrug of the compound or its pharmaceutically acceptable salt.

In some embodiments of the methods and uses disclosed herein, the GlyT1 inhibitor is a compound having a formula of

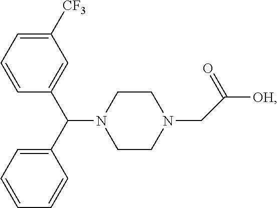 [SMILES: O=C(O)CN1CCN(C(c2ccccc2)c2cccc(C(F)(F)F)c2)CC1]

or a pharmaceutically acceptable salt thereof, or a prodrug of the compound or its pharmaceutically acceptable salt.

In some embodiments of the methods and uses disclosed herein, the GlyT1 inhibitor is a compound of Formula VI,

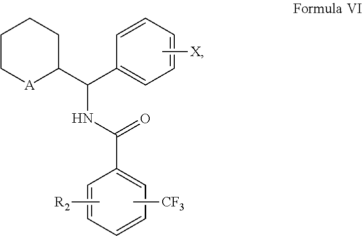

wherein:
A represents a group of general formula N—R₁, a group of general formula N+(O—)R₁ or a group of general formula N+(R′)R₁, and in which R₁ represents either a hydrogen atom, or a linear or branched (C₁-C₇)alkyl group optionally substituted with one or more fluorine atoms, or a (C₄-C₇)cycloalkyl group, or a (C₃-C₇)cycloalkyl(C₁-C₃)alkyl group, or a phenyl(C₁-C₃)alkyl group optionally substituted with one or two hydroxyl or methoxy groups, or a (C₂-C₄)alkenyl group, or a (C₂-C₄)alkynyl group,
R′ represents a linear or branched (C₁-C₇)alkyl group,
X represents a hydrogen atom or one or more substituents chosen from halogen atoms and trifluoromethyl, linear or branched (C₁-C₄)alkyl and (C₁-C₄)alkoxy groups,
R₂ represents either a hydrogen atom, or one or more substituents chosen from halogen atoms and trifluoromethyl, (C₁-C₄)alkyl or (C₁-C₄)alkoxy groups, or amino groups of general formula NR₃R₄ in which R₃ and R₄ each represent, independently of each other, a hydrogen atom or a (C₁-C₄)alkyl group, or form with the nitrogen atom carrying them a pyrrolidine, piperidine or morpholine ring, or a phenyl group optionally substituted with an atom or a group as defined for the symbol X above, or a pharmaceutically acceptable salt thereof, or a prodrug of the compound or its pharmaceutically acceptable salt.

In some embodiments of the methods and uses disclosed herein, the GlyT1 inhibitor is a compound having a formula of

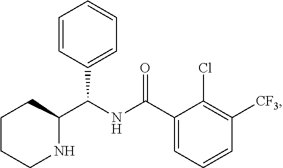 [SMILES: O=C(N[C@@H](c1ccccc1)[C@@H]1CCCCN1)c1cccc(C(F)(F)F)c1Cl]

SSR-504734, or a pharmaceutically acceptable salt thereof, or a prodrug of the compound or its pharmaceutically acceptable salt.

In some embodiments of the methods and uses disclosed herein, the GlyT1 inhibitor is a compound of Formula VII,

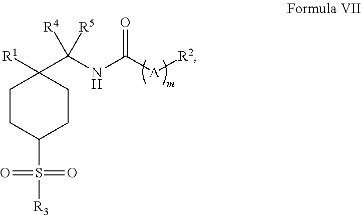

wherein:
R¹ is —(CH₂)ₙ—R^{1a}, wherein n is independently 0-6, and R^{1a} is selected from the group consisting of:
(1) C₁₋₆alkyl, which is unsubstituted or substituted with 1-6 halogen, hydroxy,
(2) phenyl substituted with R^{2a}, R^{2b} and R^{2c},
(3) C₃₋₆cycloallyl, which is unsubstituted or substituted with C₁₋₆alkyl, 1-6 halogen, hydroxy or —NR¹⁰OR¹¹,
(4) —O—C₁₋₆alkyl, which is unsubstituted or substituted with 1-6 halogen, hydroxy or —NR¹⁰R¹¹,
(5) —CO2R⁹,
wherein R9 is independently selected from:
(a) hydrogen,
(b) —C₁₋₆alkyl, which is unsubstituted or substituted with 1-6 fluoro,
(c) benzyl, and
(d) phenyl,
(6) —NR¹⁰R¹¹,
wherein R¹⁰ and R¹¹ are independently selected from:
(a) hydrogen,
(b) —C₁₋₆alkyl, which is unsubstituted or substituted with hydroxy, 1-6 fluoro or —NR¹²R¹³, where R¹² and R¹³ are independently selected from hydrogen and —C₁₋₆alkyl,
(c) —C₃₋₆cycloalkyl, which is unsubstituted or substituted with hydroxy, 1-6 fluoro or —NR¹²R¹³,
(d) benzyl,
(e) phenyl, and
(7) —CONR¹⁰R¹¹;
R² is selected from the group consisting of:
(1) phenyl, which is substituted with R^{2a}, R^{2b} and R^{2c},
(2) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c},
(3) C₃₋₆cycloalkyl, which is unsubstituted or substituted with 1-6 halogen, hydroxy or —NR¹⁰R¹¹, and
(4) —C₁₋₆alkyl-(C₃₋₆cycloalkyl), which is unsubstituted or substituted with 1-6 halogen, hydroxy or —NR¹⁰R¹¹;
R^{2a}, R^{2b} and R^{2c} are independently selected from the group consisting of:
(1) hydrogen,
(2) halogen,
(3) —C₁₋₆alkyl, which is unsubstituted or substituted with:
(a) 1-6 halogen,
(b) phenyl,
(c) C₃₋₆cycloalkyl, or
(d) —NR¹⁰R¹¹,
(4) —O—C₁₋₆alkyl, which is unsubstituted or substituted with 1-6 halogen,
(5) hydroxy,
(6) —SCF₃,
(7) —SCHF₂,
(8) —SCH₃,
(9) —CO₂R⁹,
(10) —CN,
(11) —SO₂R⁹,
(12) —SO₂—NR¹⁰R¹¹,
(13) —NR¹⁰R¹¹,
(14) —CONR¹⁰R¹¹, and
(15) —NO₂;
R³ is selected from the group consisting of:
(1) C₁₋₆alkyl, which is unsubstituted or substituted with 1-6 halogen, hydroxyl, or —NR¹⁰R¹¹,
(2) C₃₋₆cycloalkyl, which is unsubstituted or substituted with 1-6 halogen, hydroxyl or —NR¹⁰R¹¹,
R⁴ and R⁵ are independently selected from the group consisting of:
(1) hydrogen, and
(2) C₁₋₆alkyl, which is unsubstituted or substituted with halogen or hydroxyl, or R⁴ and R⁵ taken together form a C₃₋₆cycloalkyl ring;
A is selected from the group consisting of:
(1) —O—, and
(2) —NR¹⁰—;
m is zero or one, whereby when m is zero R² is attached directly to the carbonyl;
and pharmaceutically acceptable salts thereof and individual enantiomers and diastereomers thereof, or a pharmaceutically acceptable salt thereof, or a prodrug of the compound or its pharmaceutically acceptable salt.

In some embodiments of the methods and uses disclosed herein, the GlyT1 inhibitor is a compound having a formula of

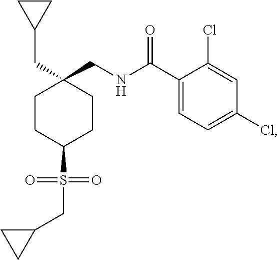 [SMILES: O=C(NC[C@]1(CC2CC2)CC[C@H](S(=O)(=O)CC2CC2)CC1)c1ccc(Cl)cc1Cl]

or a pharmaceutically acceptable salt thereof, or a prodrug of the compound or its pharmaceutically acceptable salt.

In some embodiments of the methods and uses disclosed herein, the GlyT1 inhibitor is a compound of Formula VIII,

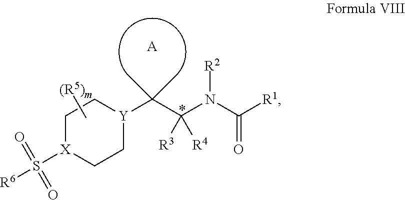

wherein:
R¹ is phenyl independently substituted from 1 to 5 times with halogen, C₁-C₃ alkyl, C₃-C₆ cycloalkyl, OR⁹, or SR¹⁰, wherein C₁-C₃ alkyl and C₃-C₆ cycloalkyl are optionally substituted with 1 to 10 times with R⁷;
R² is H;
R³ and R⁴ are each individually H or CH₃;
R⁵ is selected from the group consisting of:
(1) hydrogen,
(2) C₁-C₆ alkyl which is optionally substituted from 1 to 11 times with R⁷,
(3) gem-dialkyl, and
(4) gem-dihalo; or
two R⁵ substituents on the same carbon, together with the carbon atom to which they are attached, may form a 3-, 4-, or 5-membered cycloalkyl optionally substituted from 1 to 10 times with R⁷; or
two R⁵ substituents on adjacent carbons of the ring to which they are attached, together may form a 3-, 4-, 5- or 6-membered cycloalkyl optionally substituted from 1 to 10 times with R⁷;
R⁶ is

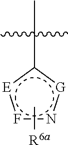

wherein E, F, and G are each independently nitrogen or carbon and R^{6a} is C₁-C₂ alkyl, which is optionally substituted 1 to 5 times with halogen or deuterium;
R⁷ is selected from the group consisting of:
(1) hydrogen,
(2) halogen,
(3) deuterium,
(4) gem-dialkyl,
(5) gem-dihalo,
(6) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰, and
(7) oxo or thio;
R⁸ is selected from the group consisting of:
(1) hydrogen,
(2) halogen,
(3) C₁-C₆ alkyl, C₂-C₆ alkenyl, C₂-C₆ alkynyl, C₃-C₇ cycloalkyl, or C₄-C₇ cycloalkylalkyl,
wherein each of the C₁-C₆ alkyl, C₂-C₆ alkenyl, C₂-C₆ alkynyl, C₃-C₇ cycloalkyl, and C₄-C₇ cycloalkylalkyl is independently and optionally substituted from 1 to 11 times with R⁷, or
(4) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰;
R⁹ is selected from the group consisting of hydrogen, C₁-C₄ alkyl, C₃-C₇ cycloalkyl, C₄-C₇ cycloalkylalkyl, —C(O)NR¹¹R¹², and —C(O)ₚR¹⁰, wherein each of C₁-C₄ alkyl, C₃-C₇ cycloalkyl, and C₄-C₇ cycloalkylalkyl is optionally substituted from 1 to 11 times with R⁷;
R¹⁰ is selected from the group consisting of hydrogen, C₁-C₄ alkyl, C₃-C₇ cycloalkyl C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R⁷ and aryl or heteroaryl is optionally substituted from 1 to 10 times with R⁸;
R¹¹ and R¹² are each independently selected from the group consisting hydrogen, C₁-C₄ alkyl, C₃-C₇ cycloalkyl, C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R¹¹ and aryl or heteroaryl is optionally substituted from 1 to 10 times with R⁸, or R¹¹ and R¹² are taken together with the nitrogen to which they are attached to form a saturated or partially saturated monocyclic or fused bicyclic heterocycle optionally substituted from 1 to 11 times with R¹¹;
A is

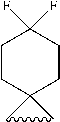

X is N;
Y is N;
p is 1, or 2; and
m is 0;
with the following provisos that: R⁶ cannot be (a) 1H-1,2,3-triazol-4-yl, or (b) 5-methylisoxazol-4-yl;
or an oxide thereof, a pharmaceutically acceptable salt of the compound or its oxide, or an individual enantiomer or diastereomer thereof.

In some embodiments of the methods and uses disclosed herein, the GlyT1 inhibitor is selected from any of the following:

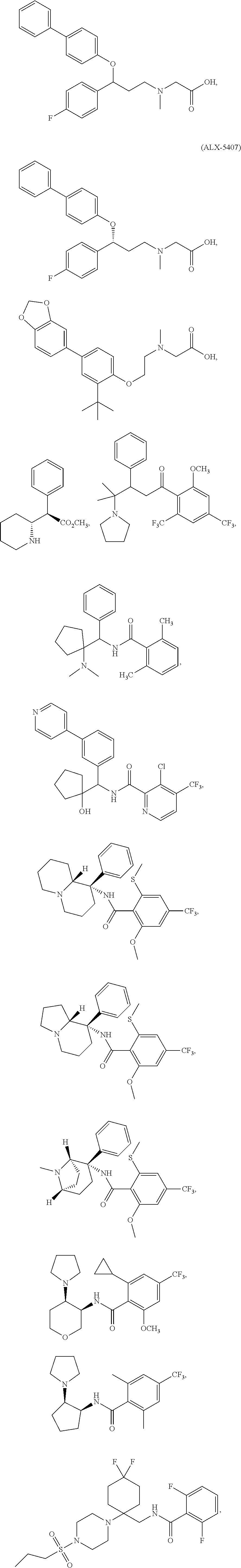

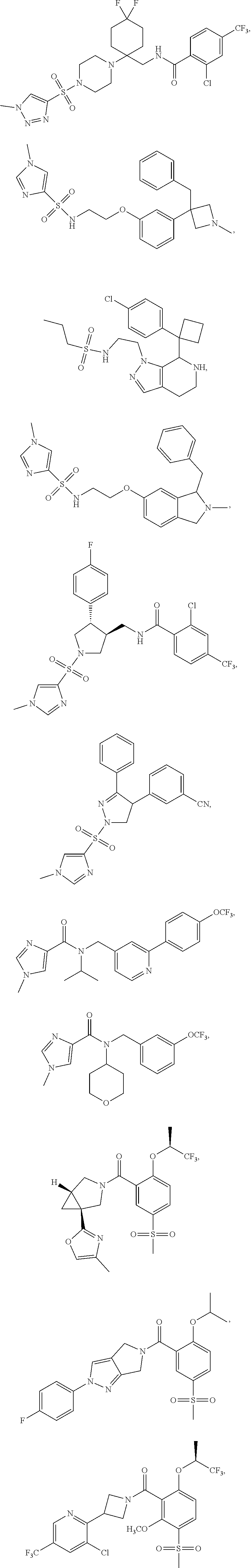

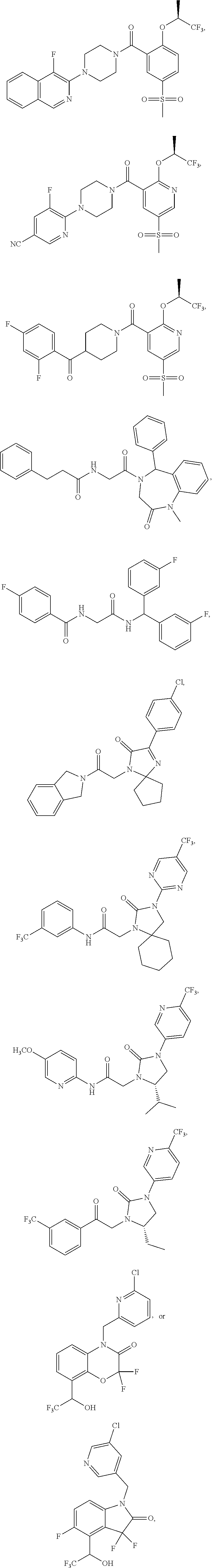

or a pharmaceutically acceptable salt thereof, or a prodrug of the compound or its pharmaceutically acceptable salt.

In some embodiments of the methods and uses disclosed herein, the GlyT1 inhibitor is a compound having a formula of

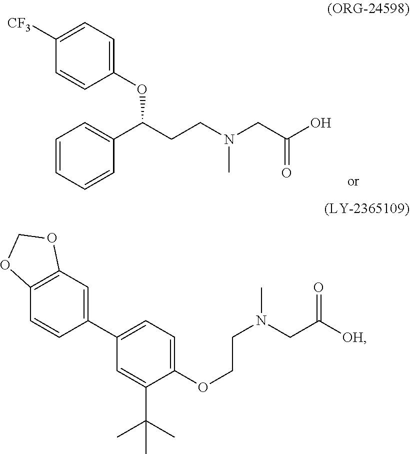 [SMILES: CN(CC[C@@H](Oc1ccc(C(F)(F)F)cc1)c1ccccc1)CC(=O)O ; CN(CCOc1ccc(-c2ccc3c(c2)OCO3)cc1C(C)(C)C)CC(=O)O]

or a pharmaceutically acceptable salt thereof, or a prodrug of the compound or its pharmaceutically acceptable salt.

In some embodiments of the methods and uses disclosed herein, the GlyT1 inhibitor is a compound of Formula IX,

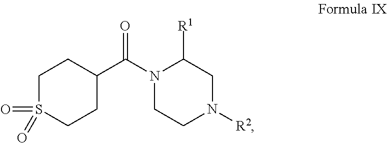

wherein:
R¹ represents phenyl or a 5 or 6 membered monocyclic heteroaryl having 1, 2, or 3 heteroatoms independently selected from O, N or S, wherein the phenyl or the heteroaryl is optionally substituted with one or more R³;
R² represents aryl, a 5 or 6 membered monocyclic heteroaryl or a 8 to 10 membered bicyclic heteroaryl, the mono- or bicyclic heteroaryl having 1, 2, or 3 heteroatoms independently selected from O, N or S, wherein the aryl or the heteroaryl is optionally substituted with one or more R⁴;
R³ is a halogen, a C₁₋₄-alkyl or a C₃₋₆-cycloalkyl, wherein the C₁₋₄-alkyl or the C₃₋₆-cycloalkyl is optionally substituted with one or more halogens; and
R⁴ is a halogen, —CN, C₁₋₄-alkyl, C₃₋₆-cycloalkyl, —C₁₋₃-alkyl —C₃₋₆-cycloalkyl or —O—C₁₋₆ alkyl, wherein the C₁₋₄-alkyl, C₃₋₆-cycloalkyl, —C₁₋₃-alkyl —C₃₋₆-cycloalkyl or the —O—C₁₋₆-alkyl is optionally substituted with one or more halogens;
or a pharmaceutically acceptable salt thereof, or a tautomer or stereoisomer of the compound or its pharmaceutically acceptable salt, or a mixture of any of the foregoing.

In certain embodiments, the compound of Formula IX can be represented by a compound of formula IX(a):

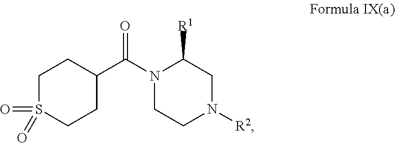

or a pharmaceutically acceptable salt thereof, or a tautomer the compound or its pharmaceutically acceptable salt, or a mixture of any of the foregoing.

In certain embodiments, the compound of Formula IX can be represented by a compound of formula IX(b):

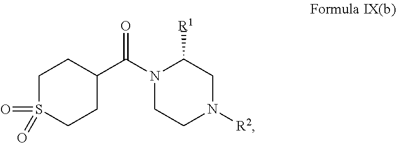

or a pharmaceutically acceptable salt thereof, or a tautomer the compound or its pharmaceutically acceptable salt, or a mixture of any of the foregoing.

In certain embodiments, the compound of formula IX is a compound selected from any of the following, a stereoisomer or stereoisomeric mixture thereof, or a pharmaceutically acceptable salt thereof:

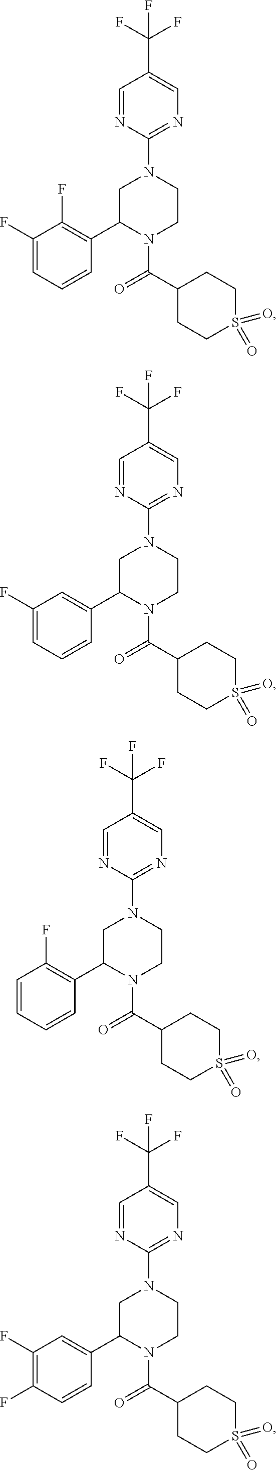 [SMILES: O=C(C1CCS(=O)(=O)CC1)N1CCN(c2ncc(C(F)(F)F)cn2)CC1c1cccc(F)c1F ; O=C(C1CCS(=O)(=O)CC1)N1CCN(c2ncc(C(F)(F)F)cn2)CC1c1cccc(F)c1 ; O=C(C1CCS(=O)(=O)CC1)N1CCN(c2ncc(C(F)(F)F)cn2)CC1c1ccccc1F ; O=C(C1CCS(=O)(=O)CC1)N1CCN(c2ncc(C(F)(F)F)cn2)CC1c1ccc(F)c(F)c1]

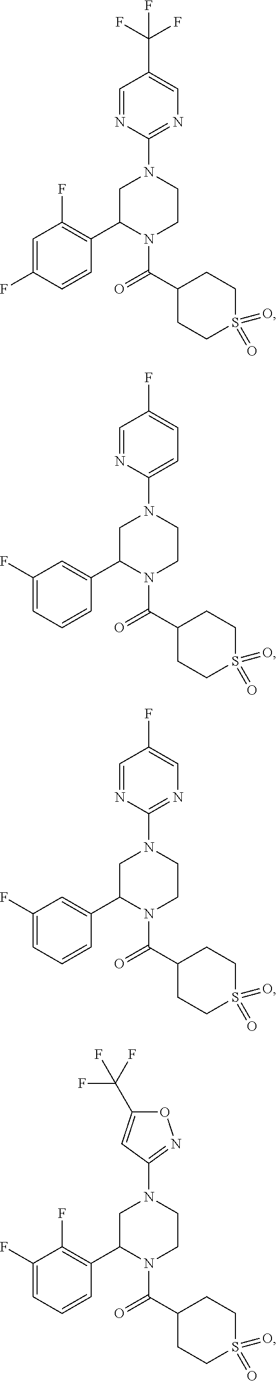 [SMILES: O=C(C1CCS(=O)(=O)CC1)N1CCN(c2ncc(C(F)(F)F)cn2)CC1c1ccc(F)cc1F ; O=C(C1CCS(=O)(=O)CC1)N1CCN(c2ccc(F)cn2)CC1c1cccc(F)c1 ; O=C(C1CCS(=O)(=O)CC1)N1CCN(c2ncc(F)cn2)CC1c1cccc(F)c1 ; O=C(C1CCS(=O)(=O)CC1)N1CCN(c2cc(C(F)(F)F)on2)CC1c1cccc(F)c1F]

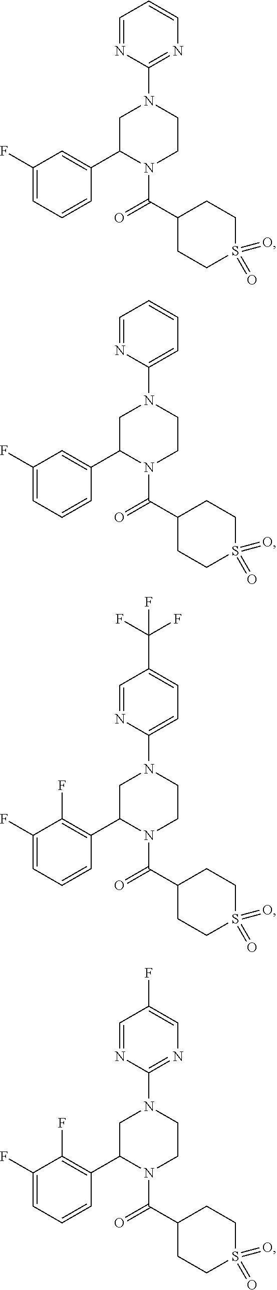 [SMILES: O=C(C1CCS(=O)(=O)CC1)N1CCN(c2ncccn2)CC1c1cccc(F)c1 ; O=C(C1CCS(=O)(=O)CC1)N1CCN(c2ccccn2)CC1c1cccc(F)c1 ; O=C(C1CCS(=O)(=O)CC1)N1CCN(c2ccc(C(F)(F)F)cn2)CC1c1cccc(F)c1F ; O=C(C1CCS(=O)(=O)CC1)N1CCN(c2ncc(F)cn2)CC1c1cccc(F)c1F]

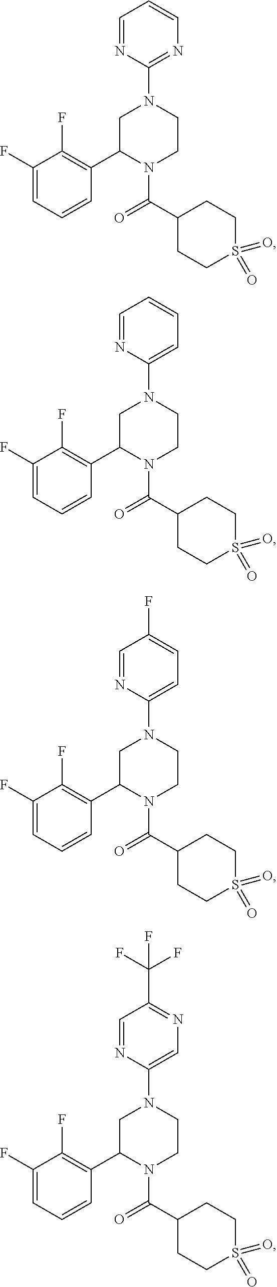 [SMILES: O=C(C1CCS(=O)(=O)CC1)N1CCN(c2ncccn2)CC1c1cccc(F)c1F ; O=C(C1CCS(=O)(=O)CC1)N1CCN(c2ccccn2)CC1c1cccc(F)c1F ; O=C(C1CCS(=O)(=O)CC1)N1CCN(c2ccc(F)cn2)CC1c1cccc(F)c1F ; O=C(C1CCS(=O)(=O)CC1)N1CCN(c2cnc(C(F)(F)F)cn2)CC1c1cccc(F)c1F]

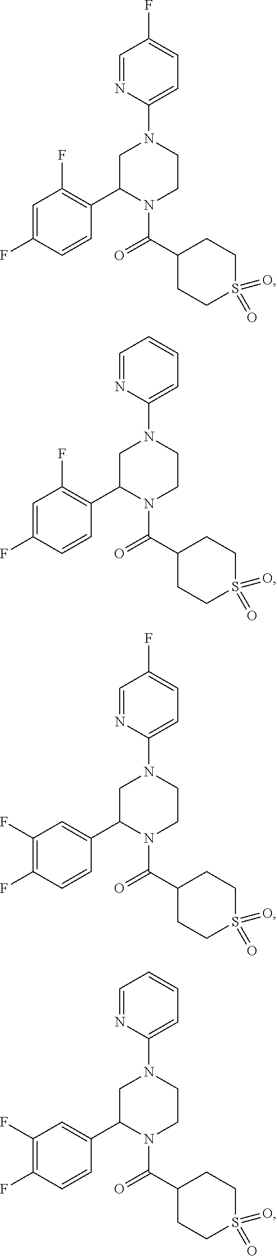 [SMILES: O=C(C1CCS(=O)(=O)CC1)N1CCN(c2ccc(F)cn2)CC1c1ccc(F)cc1F ; O=C(C1CCS(=O)(=O)CC1)N1CCN(c2ccccn2)CC1c1ccc(F)cc1F ; O=C(C1CCS(=O)(=O)CC1)N1CCN(c2ccc(F)cn2)CC1c1ccc(F)c(F)c1 ; O=C(C1CCS(=O)(=O)CC1)N1CCN(c2ccccn2)CC1c1ccc(F)c(F)c1]

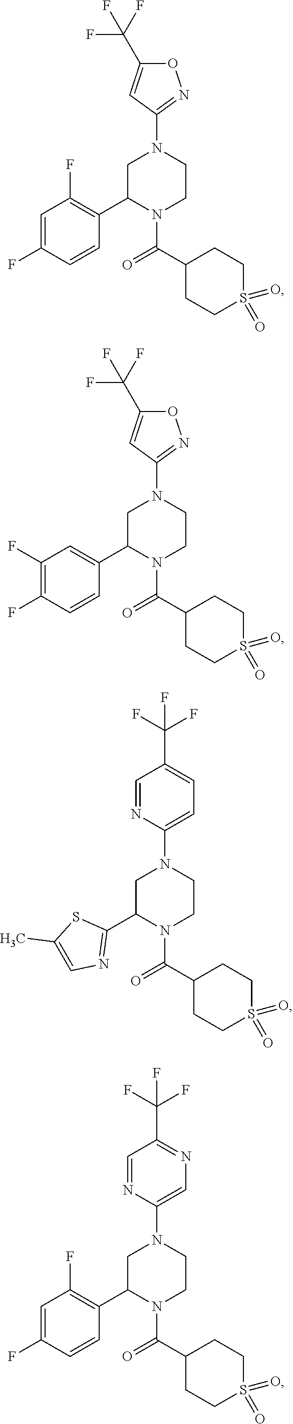 [SMILES: O=C(C1CCS(=O)(=O)CC1)N1CCN(c2cc(C(F)(F)F)on2)CC1c1ccc(F)cc1F ; O=C(C1CCS(=O)(=O)CC1)N1CCN(c2cc(C(F)(F)F)on2)CC1c1ccc(F)c(F)c1 ; Cc1cnc(C2CN(c3ccc(C(F)(F)F)cn3)CCN2C(=O)C2CCS(=O)(=O)CC2)s1 ; O=C(C1CCS(=O)(=O)CC1)N1CCN(c2cnc(C(F)(F)F)cn2)CC1c1ccc(F)cc1F]

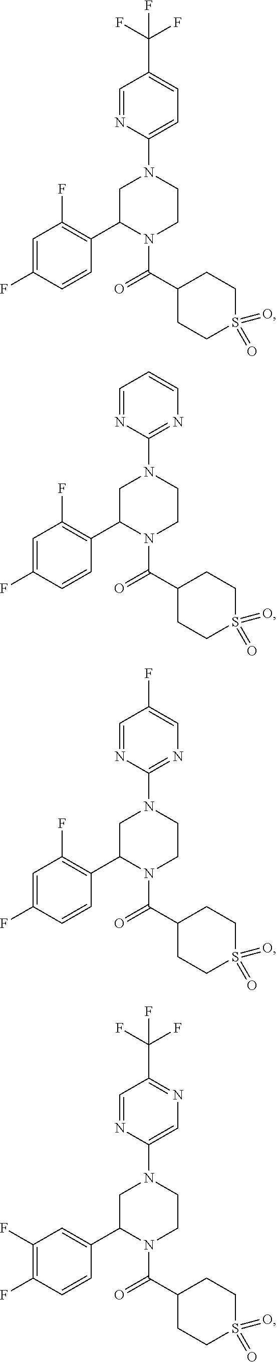 [SMILES: O=C(C1CCS(=O)(=O)CC1)N1CCN(c2ccc(C(F)(F)F)cn2)CC1c1ccc(F)cc1F ; O=C(C1CCS(=O)(=O)CC1)N1CCN(c2ncccn2)CC1c1ccc(F)cc1F ; O=C(C1CCS(=O)(=O)CC1)N1CCN(c2ncc(F)cn2)CC1c1ccc(F)cc1F ; O=C(C1CCS(=O)(=O)CC1)N1CCN(c2cnc(C(F)(F)F)cn2)CC1c1ccc(F)c(F)c1]

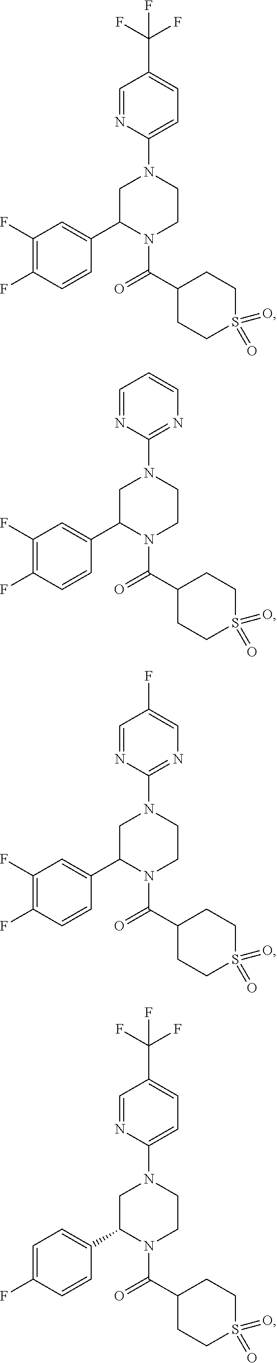 [SMILES: O=C(C1CCS(=O)(=O)CC1)N1CCN(c2ccc(C(F)(F)F)cn2)CC1c1ccc(F)c(F)c1 ; O=C(C1CCS(=O)(=O)CC1)N1CCN(c2ncccn2)CC1c1ccc(F)c(F)c1 ; O=C(C1CCS(=O)(=O)CC1)N1CCN(c2ncc(F)cn2)CC1c1ccc(F)c(F)c1 ; O=C(C1CCS(=O)(=O)CC1)N1CCN(c2ccc(C(F)(F)F)cn2)C[C@H]1c1ccc(F)cc1]

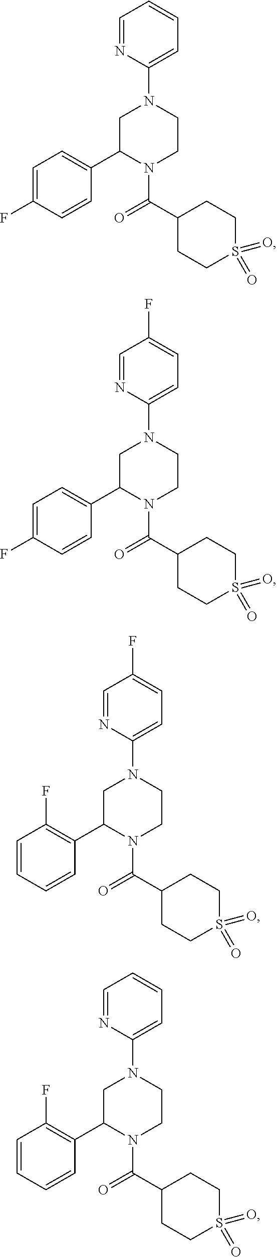 [SMILES: O=C(C1CCS(=O)(=O)CC1)N1CCN(c2ccccn2)CC1c1ccc(F)cc1 ; O=C(C1CCS(=O)(=O)CC1)N1CCN(c2ccc(F)cn2)CC1c1ccc(F)cc1 ; O=C(C1CCS(=O)(=O)CC1)N1CCN(c2ccc(F)cn2)CC1c1ccccc1F ; O=C(C1CCS(=O)(=O)CC1)N1CCN(c2ccccn2)CC1c1ccccc1F]

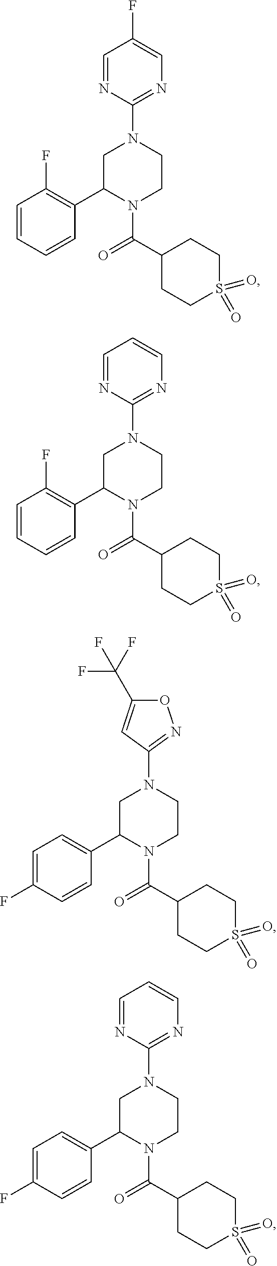 [SMILES: O=C(C1CCS(=O)(=O)CC1)N1CCN(c2ncc(F)cn2)CC1c1ccccc1F ; O=C(C1CCS(=O)(=O)CC1)N1CCN(c2ncccn2)CC1c1ccccc1F ; O=C(C1CCS(=O)(=O)CC1)N1CCN(c2cc(C(F)(F)F)on2)CC1c1ccc(F)cc1 ; O=C(C1CCS(=O)(=O)CC1)N1CCN(c2ncccn2)CC1c1ccc(F)cc1]

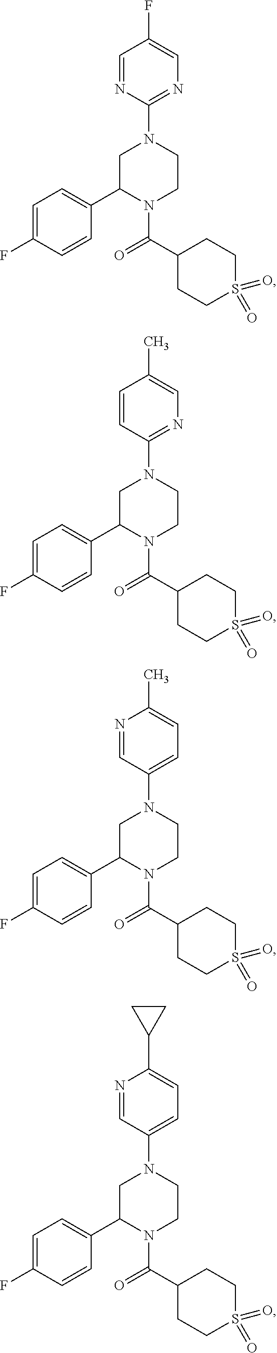 [SMILES: O=C(C1CCS(=O)(=O)CC1)N1CCN(c2ncc(F)cn2)CC1c1ccc(F)cc1 ; Cc1ccc(N2CCN(C(=O)C3CCS(=O)(=O)CC3)C(c3ccc(F)cc3)C2)nc1 ; Cc1ccc(N2CCN(C(=O)C3CCS(=O)(=O)CC3)C(c3ccc(F)cc3)C2)cn1 ; O=C(C1CCS(=O)(=O)CC1)N1CCN(c2ccc(C3CC3)nc2)CC1c1ccc(F)cc1]

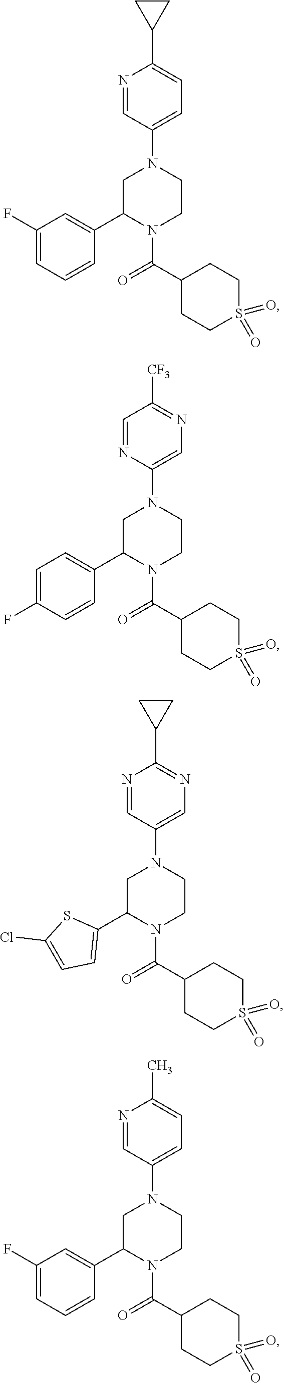 [SMILES: O=C(C1CCS(=O)(=O)CC1)N1CCN(c2ccc(C3CC3)nc2)CC1c1cccc(F)c1 ; O=C(C1CCS(=O)(=O)CC1)N1CCN(c2cnc(C(F)(F)F)cn2)CC1c1ccc(F)cc1 ; O=C(C1CCS(=O)(=O)CC1)N1CCN(c2cnc(C3CC3)nc2)CC1c1ccc(Cl)s1 ; Cc1ccc(N2CCN(C(=O)C3CCS(=O)(=O)CC3)C(c3cccc(F)c3)C2)cn1]

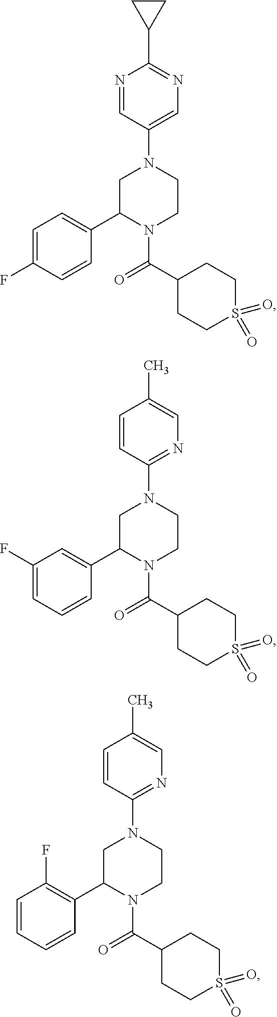 [SMILES: O=C(C1CCS(=O)(=O)CC1)N1CCN(c2cnc(C3CC3)nc2)CC1c1ccc(F)cc1 ; Cc1ccc(N2CCN(C(=O)C3CCS(=O)(=O)CC3)C(c3cccc(F)c3)C2)nc1 ; Cc1ccc(N2CCN(C(=O)C3CCS(=O)(=O)CC3)C(c3ccccc3F)C2)nc1]

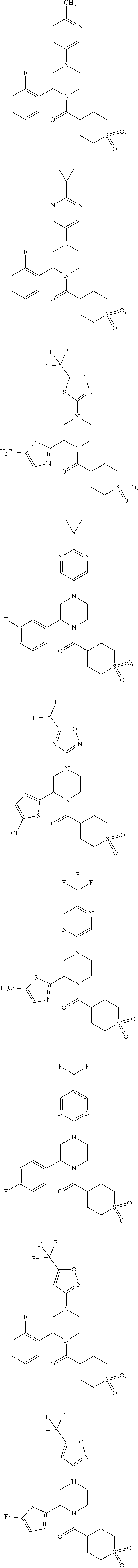

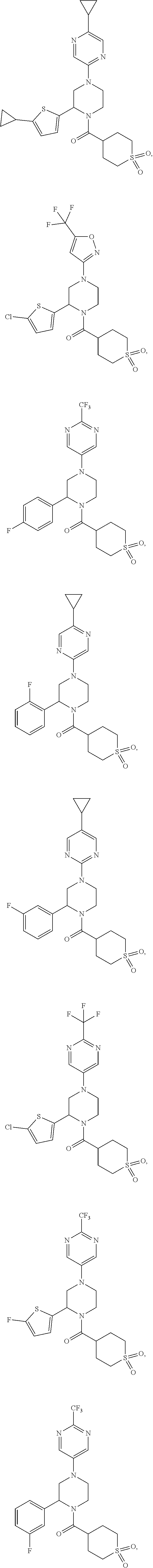

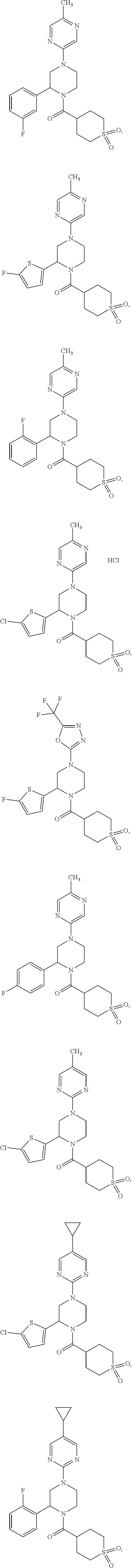

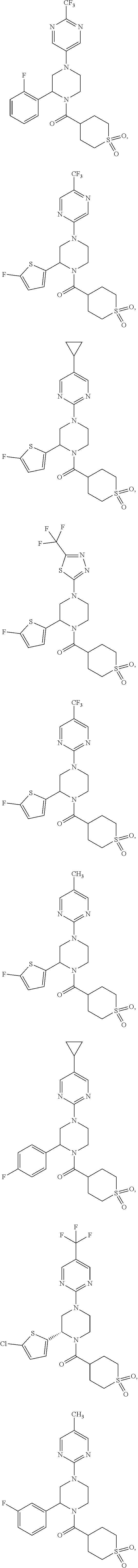

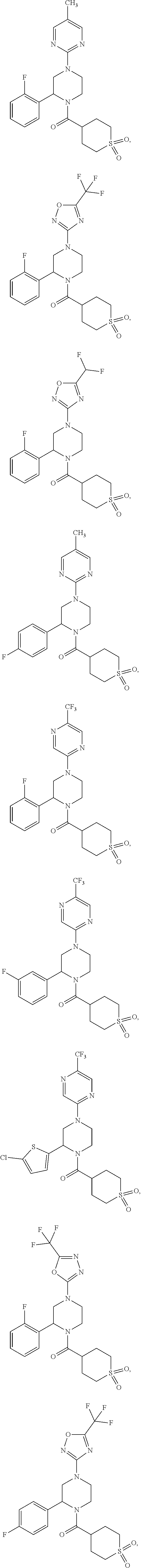

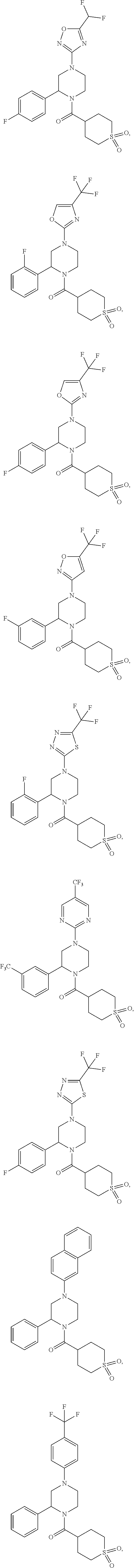

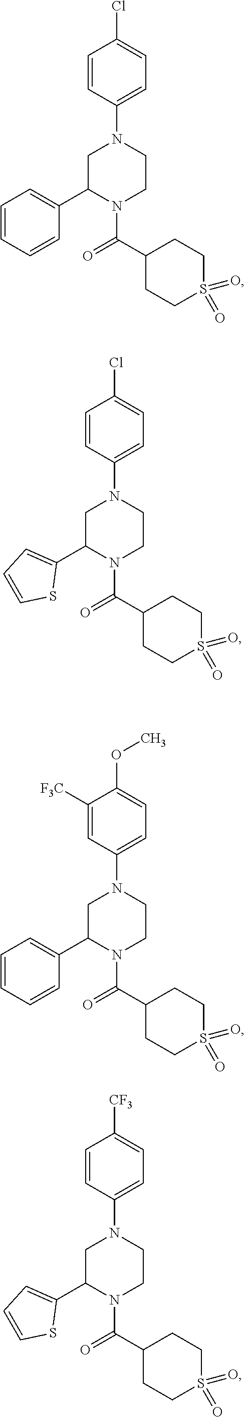 [SMILES: O=C(C1CCS(=O)(=O)CC1)N1CCN(c2ccc(Cl)cc2)CC1c1ccccc1 ; O=C(C1CCS(=O)(=O)CC1)N1CCN(c2ccc(Cl)cc2)CC1c1cccs1 ; COc1ccc(N2CCN(C(=O)C3CCS(=O)(=O)CC3)C(c3ccccc3)C2)cc1C(F)(F)F ; O=C(C1CCS(=O)(=O)CC1)N1CCN(c2ccc(C(F)(F)F)cc2)CC1c1cccs1]

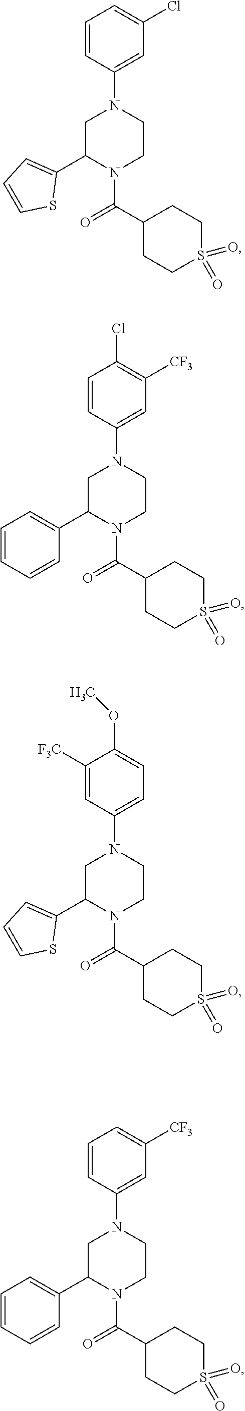 [SMILES: O=C(C1CCS(=O)(=O)CC1)N1CCN(c2cccc(Cl)c2)CC1c1cccs1 ; O=C(C1CCS(=O)(=O)CC1)N1CCN(c2ccc(Cl)c(C(F)(F)F)c2)CC1c1ccccc1 ; COc1ccc(N2CCN(C(=O)C3CCS(=O)(=O)CC3)C(c3cccs3)C2)cc1C(F)(F)F ; O=C(C1CCS(=O)(=O)CC1)N1CCN(c2cccc(C(F)(F)F)c2)CC1c1ccccc1]

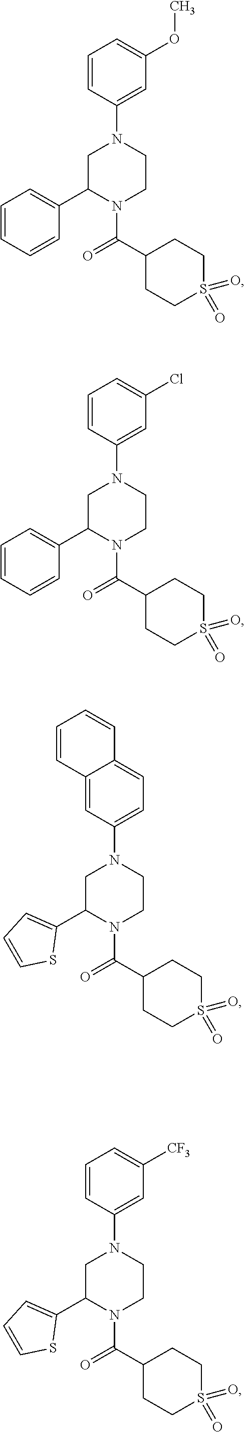 [SMILES: COc1cccc(N2CCN(C(=O)C3CCS(=O)(=O)CC3)C(c3ccccc3)C2)c1 ; O=C(C1CCS(=O)(=O)CC1)N1CCN(c2cccc(Cl)c2)CC1c1ccccc1 ; O=C(C1CCS(=O)(=O)CC1)N1CCN(c2ccc3ccccc3c2)CC1c1cccs1 ; O=C(C1CCS(=O)(=O)CC1)N1CCN(c2cccc(C(F)(F)F)c2)CC1c1cccs1]

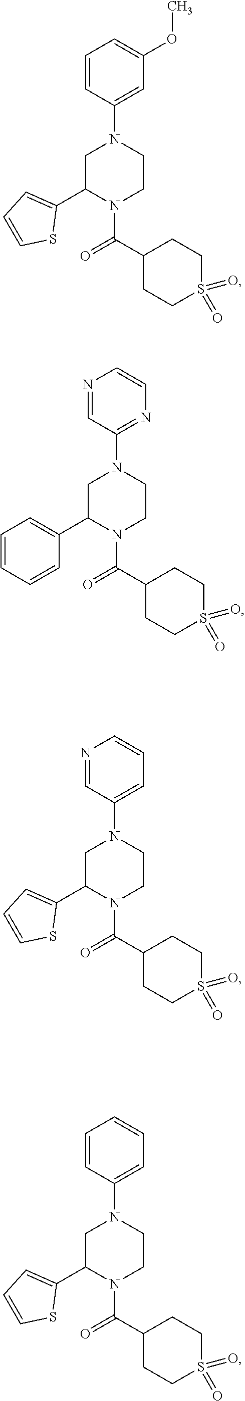 [SMILES: COc1cccc(N2CCN(C(=O)C3CCS(=O)(=O)CC3)C(c3cccs3)C2)c1 ; O=C(C1CCS(=O)(=O)CC1)N1CCN(c2cnccn2)CC1c1ccccc1 ; O=C(C1CCS(=O)(=O)CC1)N1CCN(c2cccnc2)CC1c1cccs1 ; O=C(C1CCS(=O)(=O)CC1)N1CCN(c2ccccc2)CC1c1cccs1]

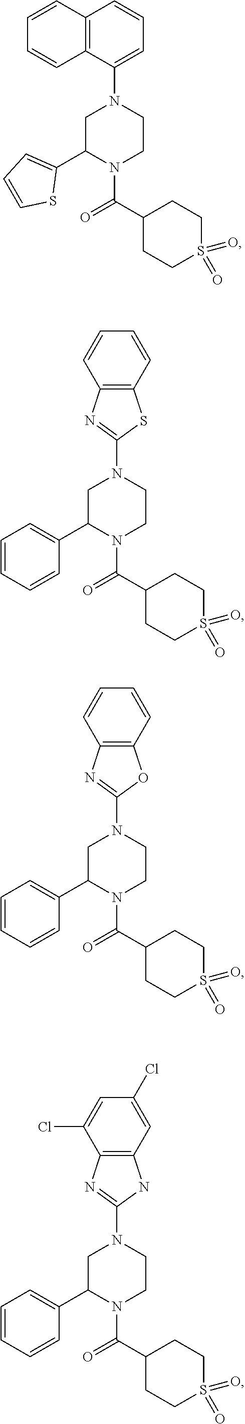 [SMILES: O=C(C1CCS(=O)(=O)CC1)N1CCN(c2cccc3ccccc23)CC1c1cccs1 ; O=C(C1CCS(=O)(=O)CC1)N1CCN(c2nc3ccccc3s2)CC1c1ccccc1 ; O=C(C1CCS(=O)(=O)CC1)N1CCN(c2nc3ccccc3o2)CC1c1ccccc1 ; O=C(C1CCS(=O)(=O)CC1)N1CCN(c2nc3c(Cl)cc(Cl)cc3[nH]2)CC1c1ccccc1]

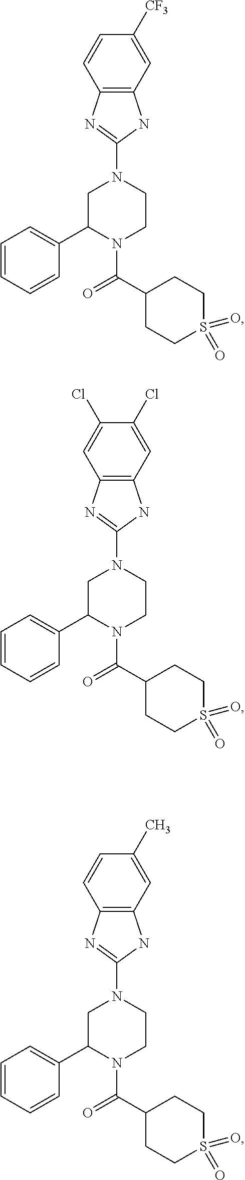 [SMILES: O=C(C1CCS(=O)(=O)CC1)N1CCN(c2nc3ccc(C(F)(F)F)cc3[nH]2)CC1c1ccccc1 ; O=C(C1CCS(=O)(=O)CC1)N1CCN(c2nc3cc(Cl)c(Cl)cc3[nH]2)CC1c1ccccc1 ; Cc1ccc2nc(N3CCN(C(=O)C4CCS(=O)(=O)CC4)C(c4ccccc4)C3)[nH]c2c1]

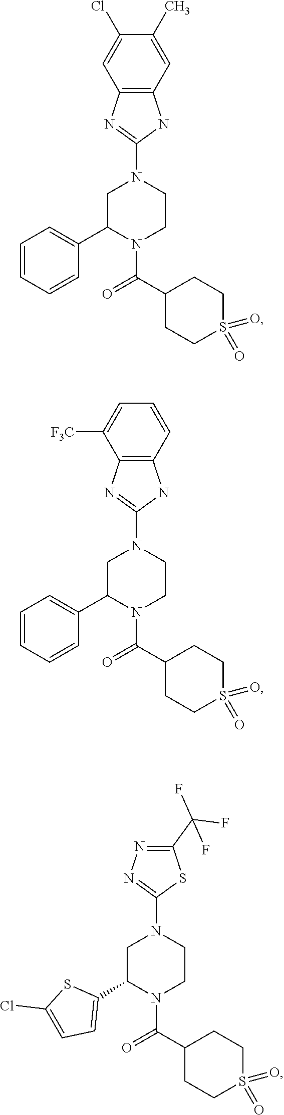 [SMILES: Cc1cc2[nH]c(N3CCN(C(=O)C4CCS(=O)(=O)CC4)C(c4ccccc4)C3)nc2cc1Cl ; O=C(C1CCS(=O)(=O)CC1)N1CCN(c2nc3c(C(F)(F)F)cccc3[nH]2)CC1c1ccccc1 ; O=C(C1CCS(=O)(=O)CC1)N1CCN(c2nnc(C(F)(F)F)s2)C[C@H]1c1ccc(Cl)s1]

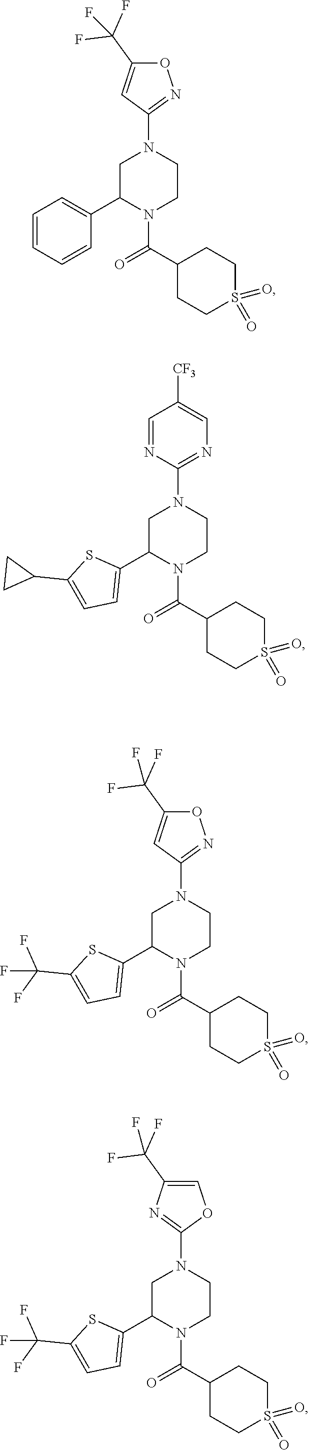 [SMILES: O=C(C1CCS(=O)(=O)CC1)N1CCN(c2cc(C(F)(F)F)on2)CC1c1ccccc1 ; O=C(C1CCS(=O)(=O)CC1)N1CCN(c2ncc(C(F)(F)F)cn2)CC1c1ccc(C2CC2)s1 ; O=C(C1CCS(=O)(=O)CC1)N1CCN(c2cc(C(F)(F)F)on2)CC1c1ccc(C(F)(F)F)s1 ; O=C(C1CCS(=O)(=O)CC1)N1CCN(c2nc(C(F)(F)F)co2)CC1c1ccc(C(F)(F)F)s1]

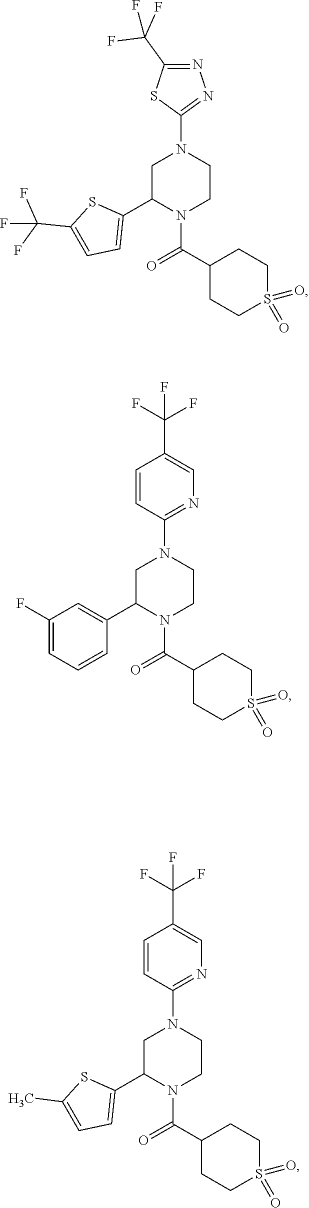 [SMILES: O=C(C1CCS(=O)(=O)CC1)N1CCN(c2nnc(C(F)(F)F)s2)CC1c1ccc(C(F)(F)F)s1 ; O=C(C1CCS(=O)(=O)CC1)N1CCN(c2ccc(C(F)(F)F)cn2)CC1c1cccc(F)c1 ; Cc1ccc(C2CN(c3ccc(C(F)(F)F)cn3)CCN2C(=O)C2CCS(=O)(=O)CC2)s1]

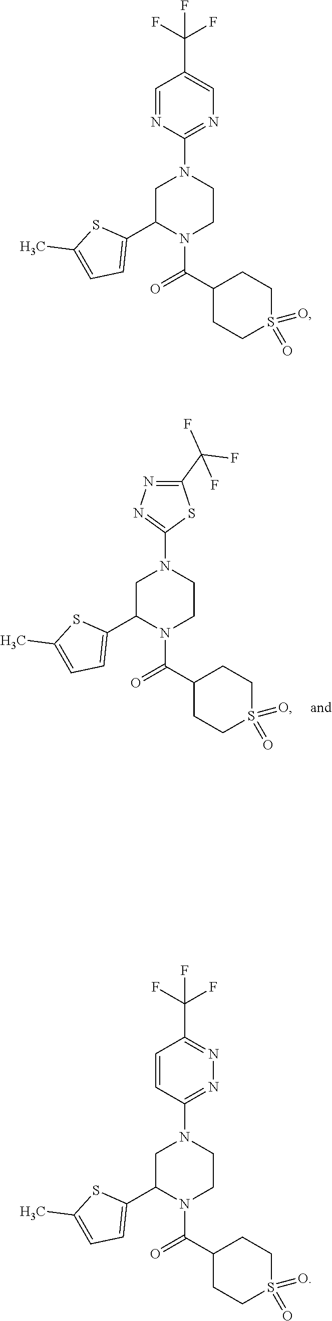 [SMILES: Cc1ccc(C2CN(c3ncc(C(F)(F)F)cn3)CCN2C(=O)C2CCS(=O)(=O)CC2)s1 ; Cc1ccc(C2CN(c3nnc(C(F)(F)F)s3)CCN2C(=O)C2CCS(=O)(=O)CC2)s1 ; Cc1ccc(C2CN(c3ccc(C(F)(F)F)nn3)CCN2C(=O)C2CCS(=O)(=O)CC2)s1]

In some embodiments of the methods and uses disclosed herein, the GlyT1 inhibitor is a compound of Formula X,

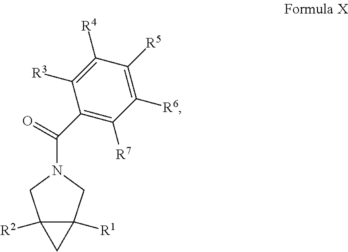

wherein:
R¹ is selected from the group consisting of
a) 5 or 6 membered monocyclic heteroaryl, having 1, 2, 3 or 4 heteroatoms independently selected from the group consisting of O, N and S(O)r,
b) 5 or 6 membered monocyclic partially saturated heterocycloalkyl, having 1, 2 or 3 heteroatoms independently selected from the group consisting of O, N and S(O)r, and
c) 9 or 10 membered bicyclic heteroaryl, having 1, 2 or 3 heteroatoms independently selected from the group consisting of O, N and S(O)ᵣ,
wherein r is 0, 1 or 2;
wherein each of said groups a), b) and c) is optionally substituted with 1 or more substituents independently selected from the group consisting of C₁₋₄-alkyl-, C₁₋₄-alkyl-O—, oxetanyl, tetrahydrofuranyl, tetrahydropyranyl, C₃₋₆-cycloalkyl- and C₃₋₆-cycloalkyl-O- and in case a substituent is attached to a nitrogen ring atom said substituent is selected from the group consisting of C₁₋₄-alkyl-, C₁₋₄-alkyl-CO—, C₃₋₆-cycloalkyl- and C₃₋₆-cycloalkyl-CO—,
and wherein each of said C₁₋₄-alkyl-, C₁₋₄-alkyl-O—, C₁₋₄-alkyl-CO—, oxetanyl, tetrahydrofuranyl, tetrahydropyranyl, C₃₋₆-cycloalkyl-, C₃₋₆-cycloalkyl-CO— or C₃₋₆-cycloalkyl-O— substituents may be substituted by 1 or more substituents independently selected from the group consisting of fluoro, —CF₃, —CHF₂, —CH₂F and —CN;
R² is selected from the group consisting of hydrogen, C₁₋₄-alkyl-, C₁₋₄-alkyl-O—, —CN and C₃₋₆-cycloalkyl-,
wherein each of said C₁₋₄-alkyl-, C₁₋₄-alkyl-O— and C₃₋₆-cycloalkyl-group may be optionally substituted with 1, 2, 3 or more substituents independently selected from the group consisting of fluoro, —CF₃, —CHF₂, —CH₂F and —CN;
R³ is selected from the group consisting of C₁₋₆-alkyl-O—, C₃₋₆-cycloalkyl-O—, morpholino, pyrazolyl and a 4 to 7 membered, monocyclic heterocycloalkyl-O— with 1 oxygen atom as ring member and optionally 1 or 2 heteroatoms independently selected from the group consisting of O, N and S(O)ₛ with s=0, 1 or 2,
wherein said C₁₋₆-alkyl-O— and said C₃₋₆-cycloalkyl-O— may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O- and C₃₋₆-cycloalkyl-O—;
R⁴ is hydrogen;
or R³ and R⁴ together with the ring atoms of the phenyl group to which they are bound may form a 4, 5 or 6 membered, monocyclic, partially saturated heterocycloalkyl or a heteroaryl each of which having 1, 2 or 3 heteroatoms independently selected from the group consisting of O, N and S(O)ₛ with s=0, 1 or 2, wherein there must be 1 ring oxygen atom that is directly attached to the ring carbon atom of said phenyl group to which R³ is attached to in general formula (I);
wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O—, C₃₋₆-cycloalkyl-O—, oxetanyl-O—, tetrahydrofuranyl-O— and tetrahydropyranyl-O—;
R⁵ is hydrogen;
R⁶ is selected from the group consisting of hydrogen, C₁₋₄-alkyl-SO₂—, C₃₋₆-cycloalkyl-SO₂ and —CN;
R⁷ is hydrogen;
or one of the pairs a) R⁶ and R⁷ or b) R⁶ and R⁵ form together with the ring atoms of the phenyl group to which they are bound, a 5 or 6 membered, partially saturated monocyclic heterocycloalkyl group having 1, 2 or 3 heteroatoms independently selected from the group consisting of O, N and S(O)ᵤ with u=0, 1 or 2, wherein there must be 1 —SO₂— member that is directly attached to the ring carbon atom of said phenyl group to which R⁶ is attached to in general formula (I),
wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₁₋₆-alkyl-O— and C₃₋₆-cycloalkyl-O—
   or a pharmaceutically acceptable salt thereof, or a prodrug of the compound or its pharmaceutically acceptable salt.

In certain embodiments, the compound of formula X is a compound selected from any of the following, a stereoisomer or stereoisomeric mixture thereof, or a pharmaceutically acceptable salt thereof:

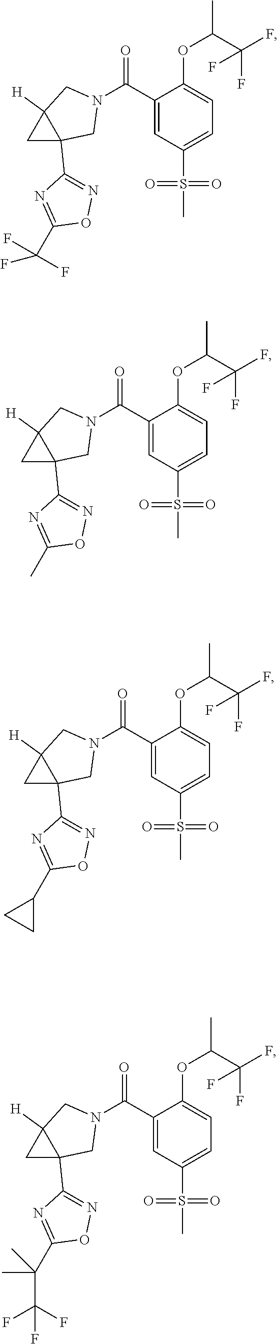 [SMILES: CC(Oc1ccc(S(C)(=O)=O)cc1C(=O)N1CC2CC2(c2noc(C(F)(F)F)n2)C1)C(F)(F)F ; Cc1nc(C23CC2CN(C(=O)c2cc(S(C)(=O)=O)ccc2OC(C)C(F)(F)F)C3)no1 ; CC(Oc1ccc(S(C)(=O)=O)cc1C(=O)N1CC2CC2(c2noc(C3CC3)n2)C1)C(F)(F)F ; CC(Oc1ccc(S(C)(=O)=O)cc1C(=O)N1CC2CC2(c2noc(C(C)(C)C(F)(F)F)n2)C1)C(F)(F)F]

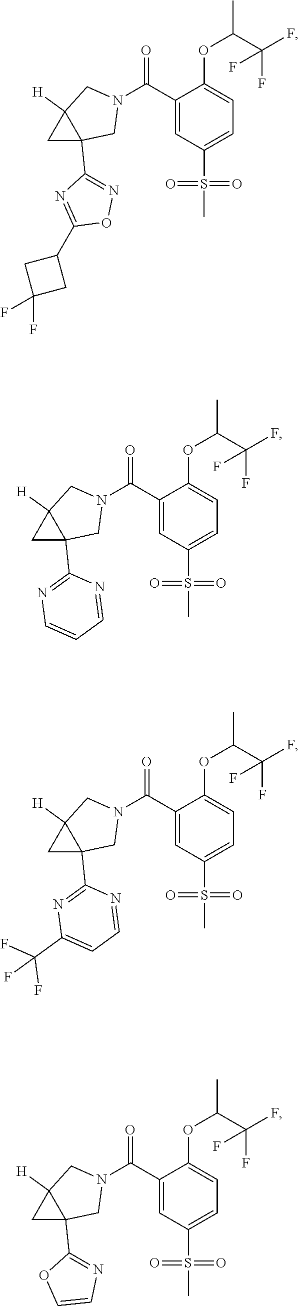 [SMILES: CC(Oc1ccc(S(C)(=O)=O)cc1C(=O)N1CC2CC2(c2noc(C3CC(F)(F)C3)n2)C1)C(F)(F)F ; CC(Oc1ccc(S(C)(=O)=O)cc1C(=O)N1CC2CC2(c2ncccn2)C1)C(F)(F)F ; CC(Oc1ccc(S(C)(=O)=O)cc1C(=O)N1CC2CC2(c2nccc(C(F)(F)F)n2)C1)C(F)(F)F ; CC(Oc1ccc(S(C)(=O)=O)cc1C(=O)N1CC2CC2(c2ncco2)C1)C(F)(F)F]

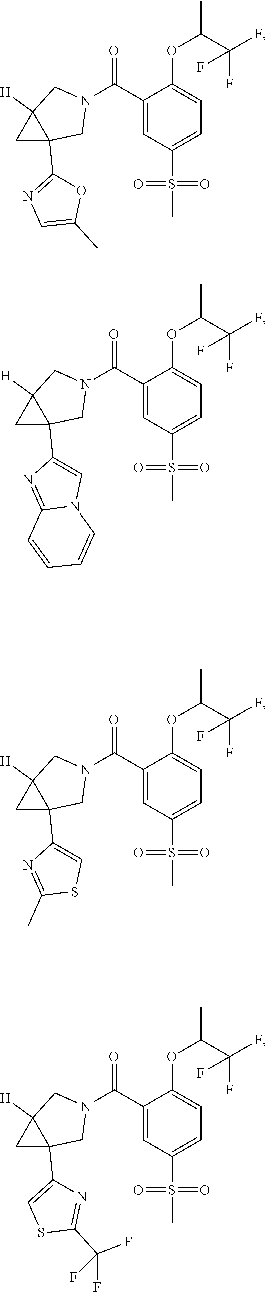 [SMILES: Cc1cnc(C23CC2CN(C(=O)c2cc(S(C)(=O)=O)ccc2OC(C)C(F)(F)F)C3)o1 ; CC(Oc1ccc(S(C)(=O)=O)cc1C(=O)N1CC2CC2(c2cn3ccccc3n2)C1)C(F)(F)F ; Cc1nc(C23CC2CN(C(=O)c2cc(S(C)(=O)=O)ccc2OC(C)C(F)(F)F)C3)cs1 ; CC(Oc1ccc(S(C)(=O)=O)cc1C(=O)N1CC2CC2(c2csc(C(F)(F)F)n2)C1)C(F)(F)F]

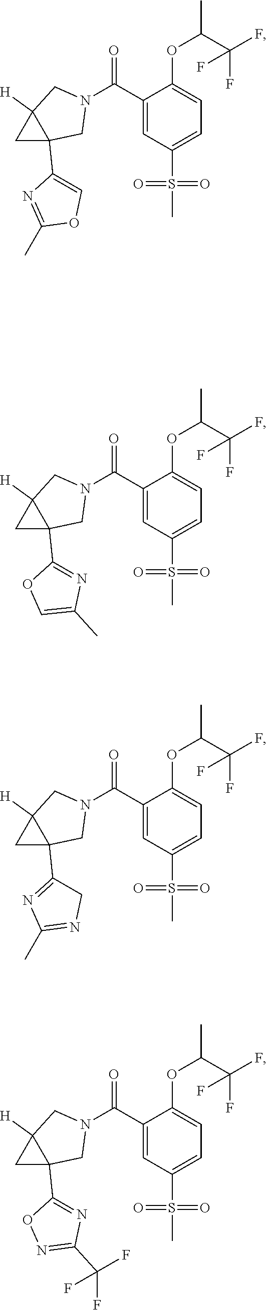 [SMILES: Cc1nc(C23CC2CN(C(=O)c2cc(S(C)(=O)=O)ccc2OC(C)C(F)(F)F)C3)co1 ; Cc1coc(C23CC2CN(C(=O)c2cc(S(C)(=O)=O)ccc2OC(C)C(F)(F)F)C3)n1 ; CC1=NCC(C23CC2CN(C(=O)c2cc(S(C)(=O)=O)ccc2OC(C)C(F)(F)F)C3)=N1 ; CC(Oc1ccc(S(C)(=O)=O)cc1C(=O)N1CC2CC2(c2nc(C(F)(F)F)no2)C1)C(F)(F)F]

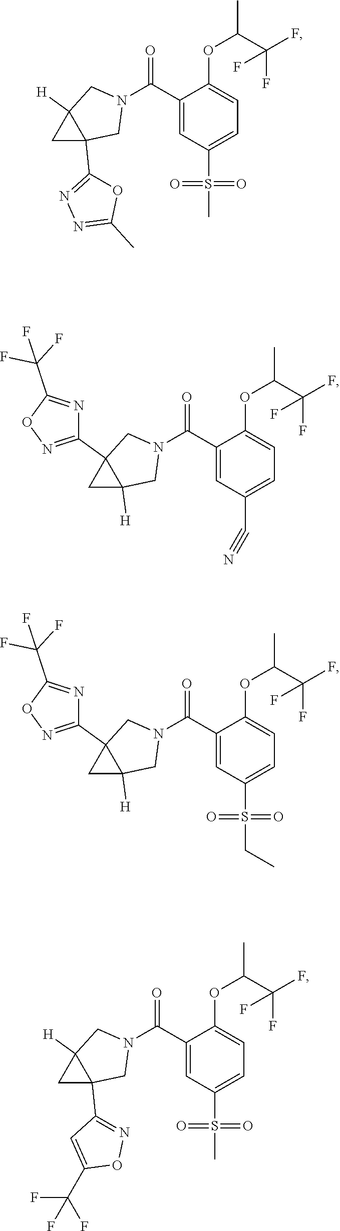 [SMILES: Cc1nnc(C23CC2CN(C(=O)c2cc(S(C)(=O)=O)ccc2OC(C)C(F)(F)F)C3)o1 ; CC(Oc1ccc(C#N)cc1C(=O)N1CC2CC2(c2noc(C(F)(F)F)n2)C1)C(F)(F)F ; CCS(=O)(=O)c1ccc(OC(C)C(F)(F)F)c(C(=O)N2CC3CC3(c3noc(C(F)(F)F)n3)C2)c1 ; CC(Oc1ccc(S(C)(=O)=O)cc1C(=O)N1CC2CC2(c2cc(C(F)(F)F)on2)C1)C(F)(F)F]

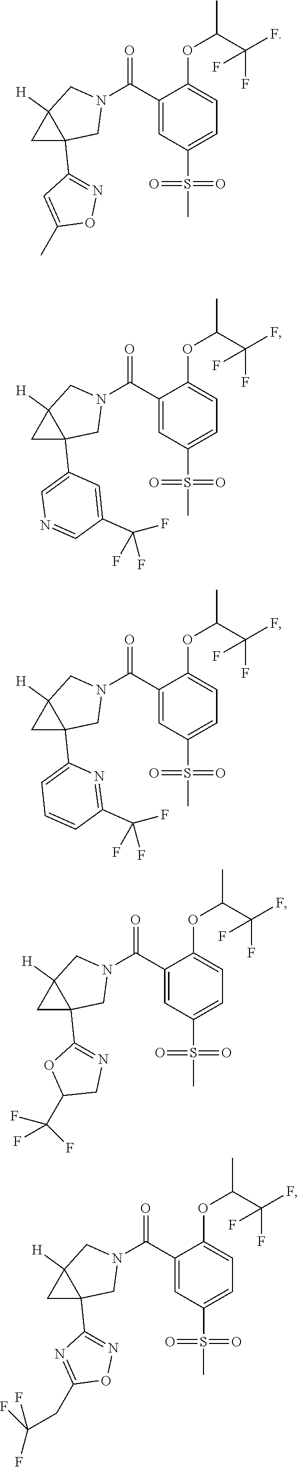 [SMILES: Cc1cc(C23CC2CN(C(=O)c2cc(S(C)(=O)=O)ccc2OC(C)C(F)(F)F)C3)no1 ; CC(Oc1ccc(S(C)(=O)=O)cc1C(=O)N1CC2CC2(c2cncc(C(F)(F)F)c2)C1)C(F)(F)F ; CC(Oc1ccc(S(C)(=O)=O)cc1C(=O)N1CC2CC2(c2cccc(C(F)(F)F)n2)C1)C(F)(F)F ; CC(Oc1ccc(S(C)(=O)=O)cc1C(=O)N1CC2CC2(C2=NCC(C(F)(F)F)O2)C1)C(F)(F)F ; CC(Oc1ccc(S(C)(=O)=O)cc1C(=O)N1CC2CC2(c2noc(CC(F)(F)F)n2)C1)C(F)(F)F]

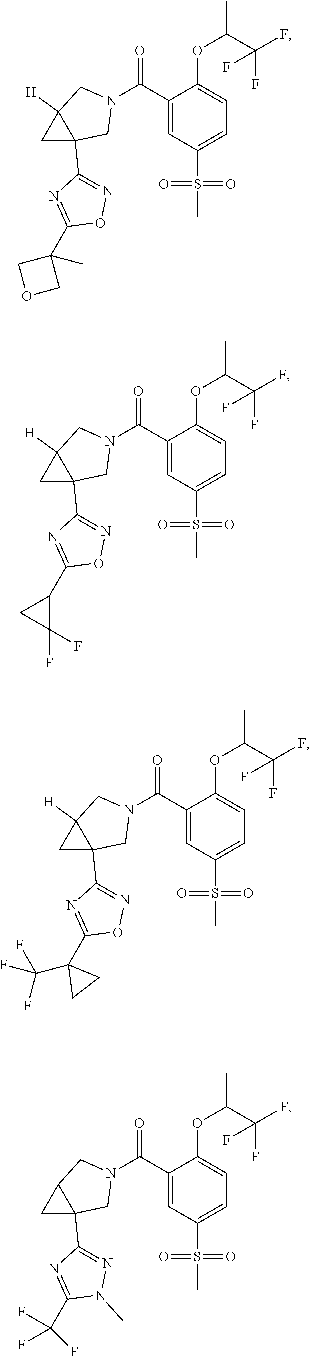 [SMILES: CC(Oc1ccc(S(C)(=O)=O)cc1C(=O)N1CC2CC2(c2noc(C3(C)COC3)n2)C1)C(F)(F)F ; CC(Oc1ccc(S(C)(=O)=O)cc1C(=O)N1CC2CC2(c2noc(C3CC3(F)F)n2)C1)C(F)(F)F ; CC(Oc1ccc(S(C)(=O)=O)cc1C(=O)N1CC2CC2(c2noc(C3(C(F)(F)F)CC3)n2)C1)C(F)(F)F ; CC(Oc1ccc(S(C)(=O)=O)cc1C(=O)N1CC2CC2(c2nc(C(F)(F)F)n(C)n2)C1)C(F)(F)F]

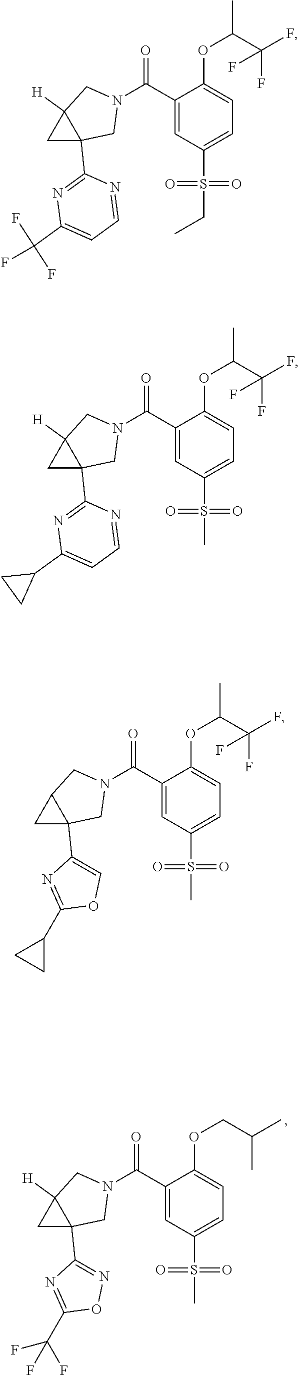 [SMILES: CCS(=O)(=O)c1ccc(OC(C)C(F)(F)F)c(C(=O)N2CC3CC3(c3nccc(C(F)(F)F)n3)C2)c1 ; CC(Oc1ccc(S(C)(=O)=O)cc1C(=O)N1CC2CC2(c2nccc(C3CC3)n2)C1)C(F)(F)F ; CC(Oc1ccc(S(C)(=O)=O)cc1C(=O)N1CC2CC2(c2coc(C3CC3)n2)C1)C(F)(F)F ; CC(C)COc1ccc(S(C)(=O)=O)cc1C(=O)N1CC2CC2(c2noc(C(F)(F)F)n2)C1 ; ","]

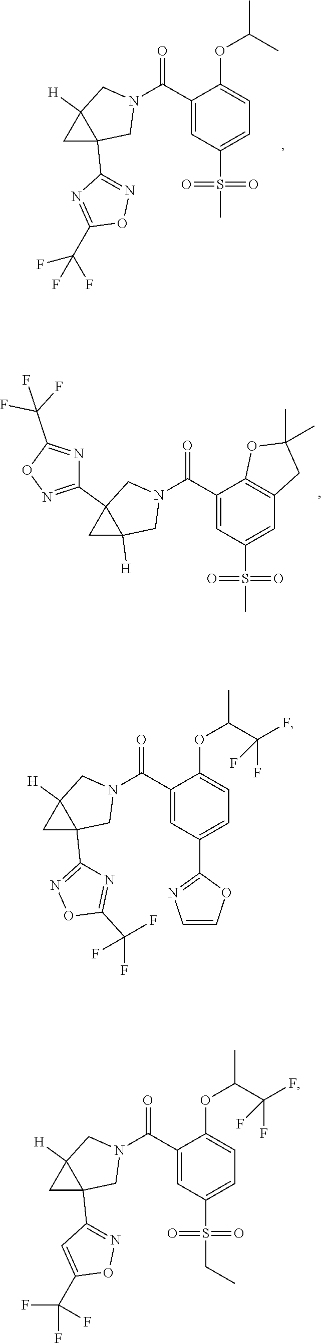 [SMILES: CC(C)Oc1ccc(S(C)(=O)=O)cc1C(=O)N1CC2CC2(c2noc(C(F)(F)F)n2)C1 ; CC1(C)Cc2cc(S(C)(=O)=O)cc(C(=O)N3CC4CC4(c4noc(C(F)(F)F)n4)C3)c2O1 ; CC(Oc1ccc(-c2ncco2)cc1C(=O)N1CC2CC2(c2noc(C(F)(F)F)n2)C1)C(F)(F)F ; CCS(=O)(=O)c1ccc(OC(C)C(F)(F)F)c(C(=O)N2CC3CC3(c3cc(C(F)(F)F)on3)C2)c1]

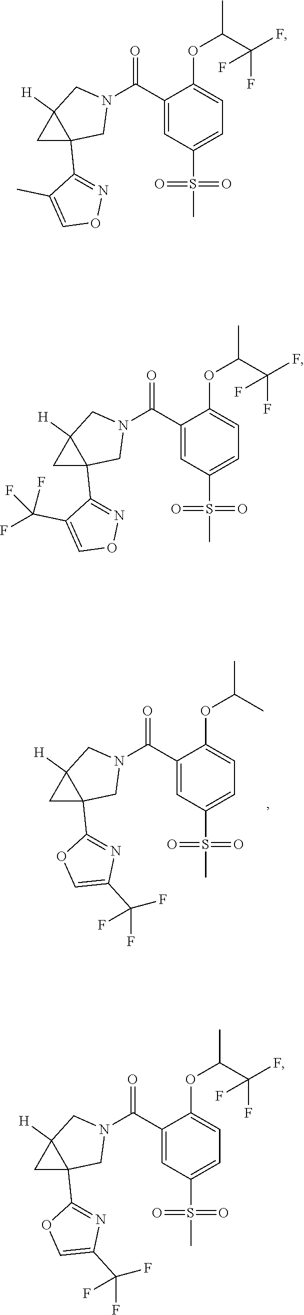 [SMILES: Cc1conc1C12CC1CN(C(=O)c1cc(S(C)(=O)=O)ccc1OC(C)C(F)(F)F)C2 ; CC(Oc1ccc(S(C)(=O)=O)cc1C(=O)N1CC2CC2(c2nocc2C(F)(F)F)C1)C(F)(F)F ; CC(C)Oc1ccc(S(C)(=O)=O)cc1C(=O)N1CC2CC2(c2nc(C(F)(F)F)co2)C1 ; CC(Oc1ccc(S(C)(=O)=O)cc1C(=O)N1CC2CC2(c2nc(C(F)(F)F)co2)C1)C(F)(F)F]

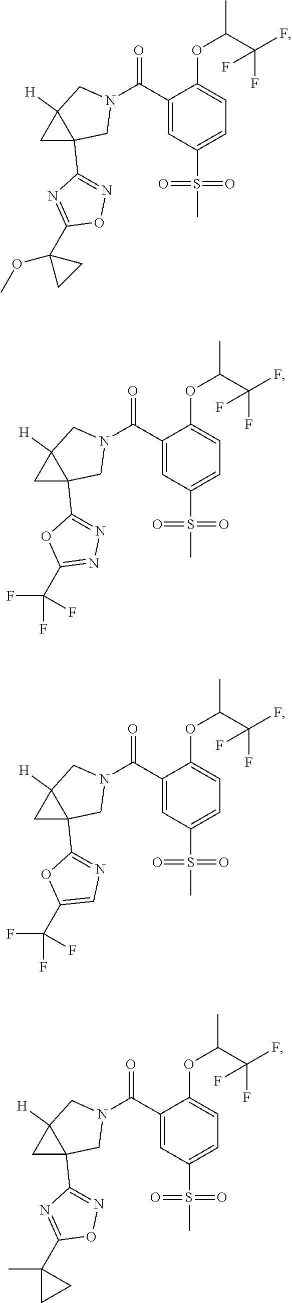 [SMILES: COC1(c2nc(C34CC3CN(C(=O)c3cc(S(C)(=O)=O)ccc3OC(C)C(F)(F)F)C4)no2)CC1 ; CC(Oc1ccc(S(C)(=O)=O)cc1C(=O)N1CC2CC2(c2nnc(C(F)(F)F)o2)C1)C(F)(F)F ; CC(Oc1ccc(S(C)(=O)=O)cc1C(=O)N1CC2CC2(c2ncc(C(F)(F)F)o2)C1)C(F)(F)F ; CC(Oc1ccc(S(C)(=O)=O)cc1C(=O)N1CC2CC2(c2noc(C3(C)CC3)n2)C1)C(F)(F)F]

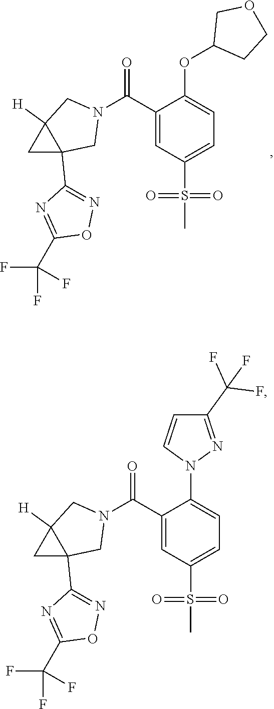 [SMILES: CS(=O)(=O)c1ccc(OC2CCOC2)c(C(=O)N2CC3CC3(c3noc(C(F)(F)F)n3)C2)c1 ; CS(=O)(=O)c1ccc(-n2ccc(C(F)(F)F)n2)c(C(=O)N2CC3CC3(c3noc(C(F)(F)F)n3)C2)c1]

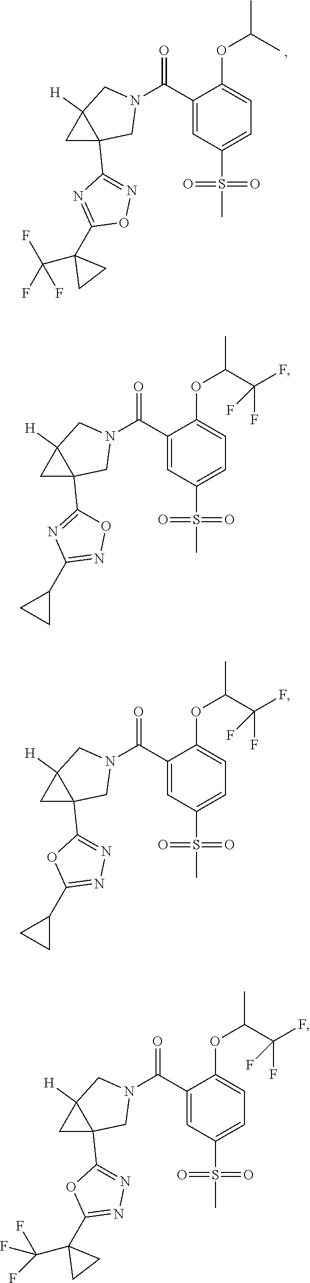 [SMILES: CC(C)Oc1ccc(S(C)(=O)=O)cc1C(=O)N1CC2CC2(c2noc(C3(C(F)(F)F)CC3)n2)C1 ; CC(Oc1ccc(S(C)(=O)=O)cc1C(=O)N1CC2CC2(c2nc(C3CC3)no2)C1)C(F)(F)F ; CC(Oc1ccc(S(C)(=O)=O)cc1C(=O)N1CC2CC2(c2nnc(C3CC3)o2)C1)C(F)(F)F ; CC(Oc1ccc(S(C)(=O)=O)cc1C(=O)N1CC2CC2(c2nnc(C3(C(F)(F)F)CC3)o2)C1)C(F)(F)F]

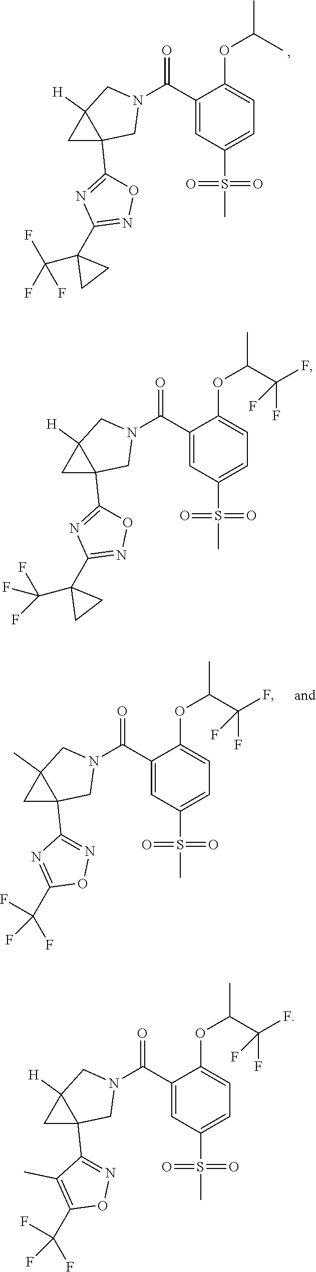

For example, the compound of Formula X could be a diastereomeric mixture or single diasteromers of any of the following, or a pharmaceutically acceptable salt thereof:

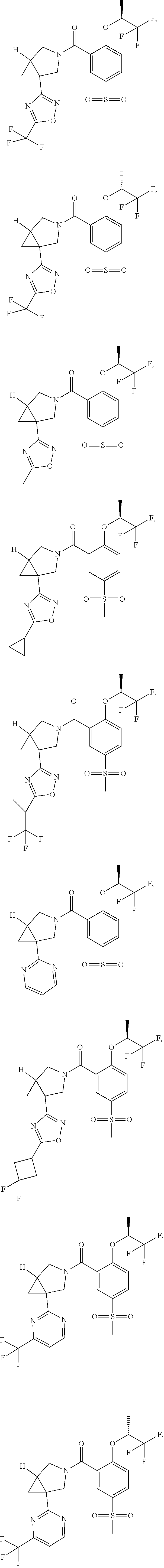

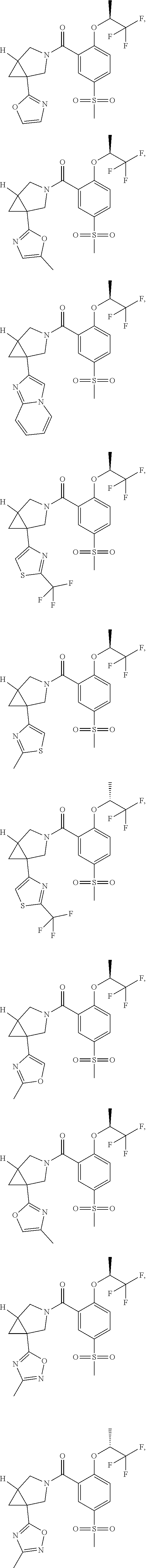

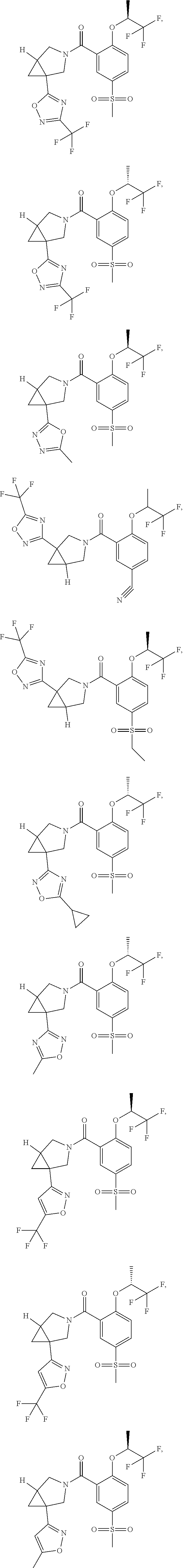

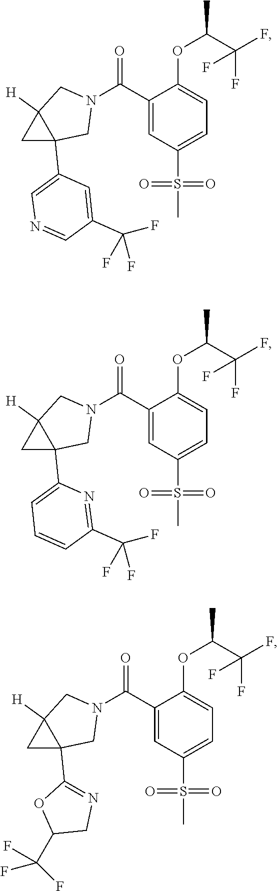 [SMILES: C[C@H](Oc1ccc(S(C)(=O)=O)cc1C(=O)N1CC2CC2(c2cncc(C(F)(F)F)c2)C1)C(F)(F)F ; C[C@H](Oc1ccc(S(C)(=O)=O)cc1C(=O)N1CC2CC2(c2cccc(C(F)(F)F)n2)C1)C(F)(F)F ; C[C@H](Oc1ccc(S(C)(=O)=O)cc1C(=O)N1CC2CC2(C2=NCC(C(F)(F)F)O2)C1)C(F)(F)F]

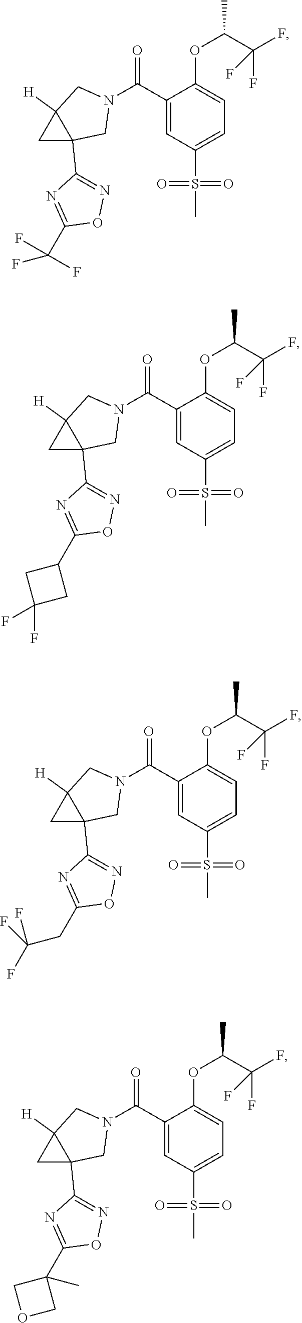 [SMILES: C[C@@H](Oc1ccc(S(C)(=O)=O)cc1C(=O)N1CC2CC2(c2noc(C(F)(F)F)n2)C1)C(F)(F)F ; C[C@H](Oc1ccc(S(C)(=O)=O)cc1C(=O)N1CC2CC2(c2noc(C3CC(F)(F)C3)n2)C1)C(F)(F)F ; C[C@H](Oc1ccc(S(C)(=O)=O)cc1C(=O)N1CC2CC2(c2noc(CC(F)(F)F)n2)C1)C(F)(F)F ; C[C@H](Oc1ccc(S(C)(=O)=O)cc1C(=O)N1CC2CC2(c2noc(C3(C)COC3)n2)C1)C(F)(F)F]

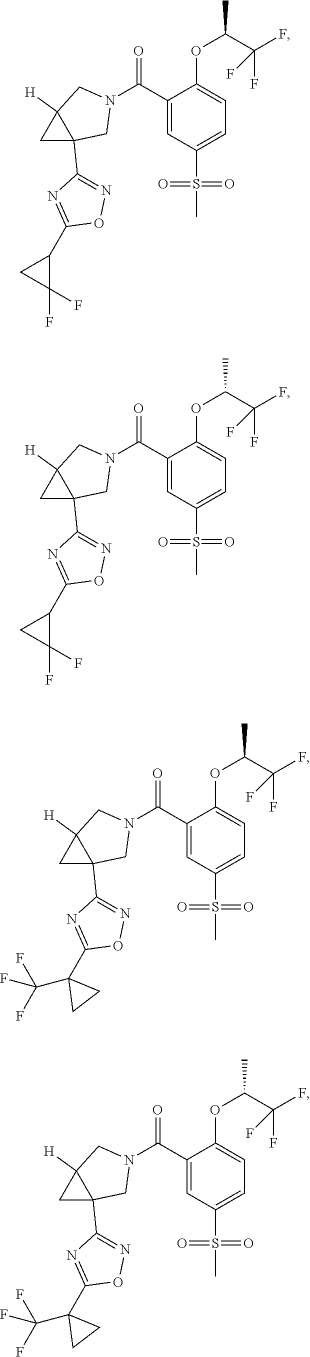 [SMILES: C[C@H](Oc1ccc(S(C)(=O)=O)cc1C(=O)N1CC2CC2(c2noc(C3CC3(F)F)n2)C1)C(F)(F)F ; C[C@@H](Oc1ccc(S(C)(=O)=O)cc1C(=O)N1CC2CC2(c2noc(C3CC3(F)F)n2)C1)C(F)(F)F ; C[C@H](Oc1ccc(S(C)(=O)=O)cc1C(=O)N1CC2CC2(c2noc(C3(C(F)(F)F)CC3)n2)C1)C(F)(F)F ; C[C@@H](Oc1ccc(S(C)(=O)=O)cc1C(=O)N1CC2CC2(c2noc(C3(C(F)(F)F)CC3)n2)C1)C(F)(F)F]

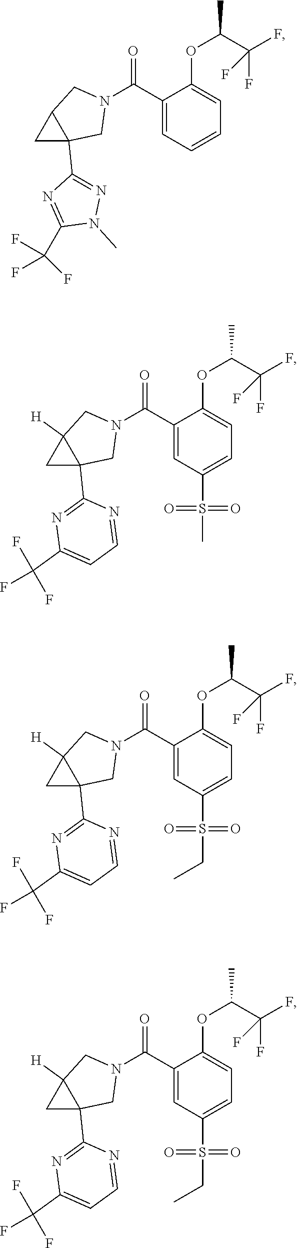 [SMILES: C[C@H](Oc1ccccc1C(=O)N1CC2CC2(c2nc(C(F)(F)F)n(C)n2)C1)C(F)(F)F ; C[C@@H](Oc1ccc(S(C)(=O)=O)cc1C(=O)N1CC2CC2(c2nccc(C(F)(F)F)n2)C1)C(F)(F)F ; CCS(=O)(=O)c1ccc(O[C@@H](C)C(F)(F)F)c(C(=O)N2CC3CC3(c3nccc(C(F)(F)F)n3)C2)c1 ; CCS(=O)(=O)c1ccc(O[C@H](C)C(F)(F)F)c(C(=O)N2CC3CC3(c3nccc(C(F)(F)F)n3)C2)c1]

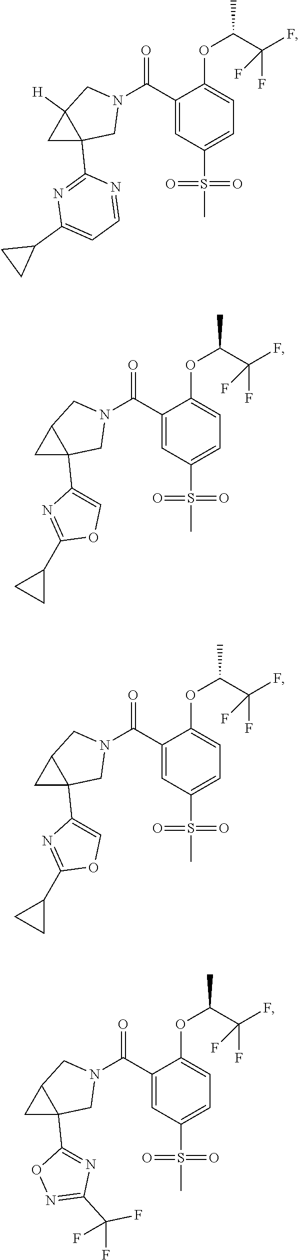 [SMILES: C[C@@H](Oc1ccc(S(C)(=O)=O)cc1C(=O)N1CC2CC2(c2nccc(C3CC3)n2)C1)C(F)(F)F ; C[C@H](Oc1ccc(S(C)(=O)=O)cc1C(=O)N1CC2CC2(c2coc(C3CC3)n2)C1)C(F)(F)F ; C[C@@H](Oc1ccc(S(C)(=O)=O)cc1C(=O)N1CC2CC2(c2coc(C3CC3)n2)C1)C(F)(F)F ; C[C@H](Oc1ccc(S(C)(=O)=O)cc1C(=O)N1CC2CC2(c2nc(C(F)(F)F)no2)C1)C(F)(F)F]

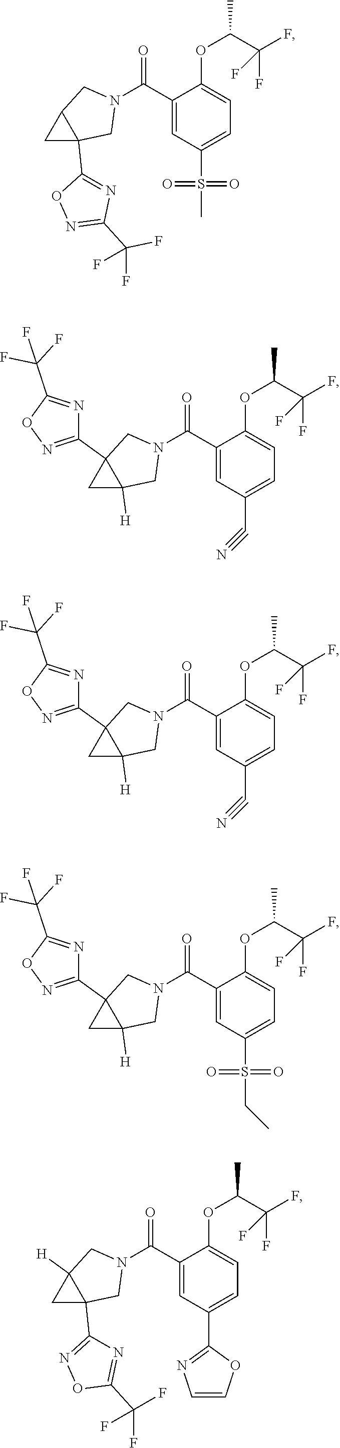

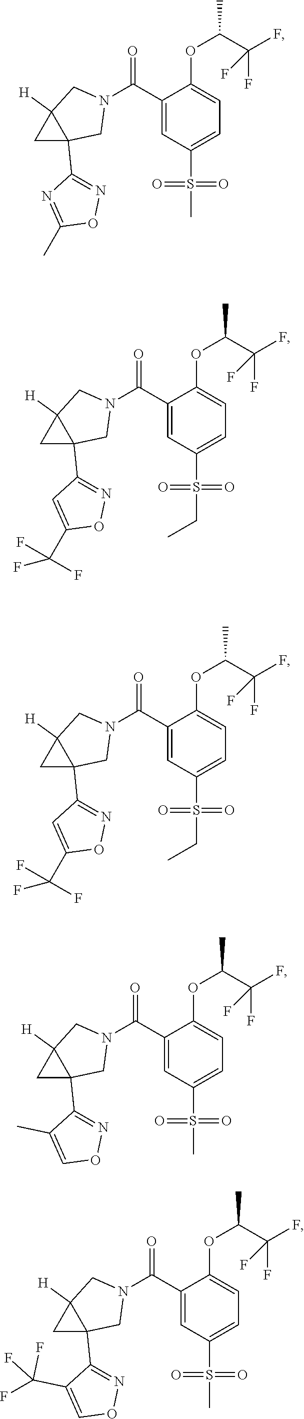 [SMILES: Cc1nc(C23CC2CN(C(=O)c2cc(S(C)(=O)=O)ccc2O[C@H](C)C(F)(F)F)C3)no1 ; CCS(=O)(=O)c1ccc(O[C@@H](C)C(F)(F)F)c(C(=O)N2CC3CC3(c3cc(C(F)(F)F)on3)C2)c1 ; CCS(=O)(=O)c1ccc(O[C@H](C)C(F)(F)F)c(C(=O)N2CC3CC3(c3cc(C(F)(F)F)on3)C2)c1 ; Cc1conc1C12CC1CN(C(=O)c1cc(S(C)(=O)=O)ccc1O[C@@H](C)C(F)(F)F)C2 ; C[C@H](Oc1ccc(S(C)(=O)=O)cc1C(=O)N1CC2CC2(c2nocc2C(F)(F)F)C1)C(F)(F)F]

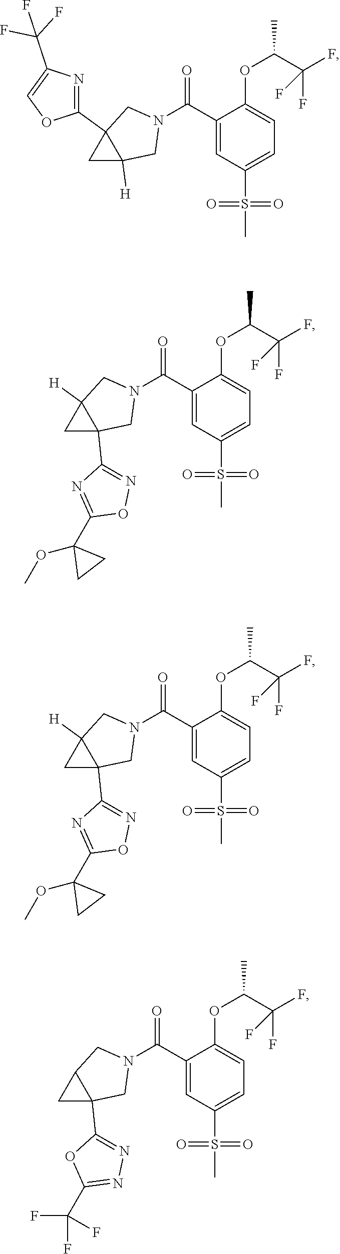 [SMILES: C[C@@H](Oc1ccc(S(C)(=O)=O)cc1C(=O)N1CC2CC2(c2nc(C(F)(F)F)co2)C1)C(F)(F)F ; COC1(c2nc(C34CC3CN(C(=O)c3cc(S(C)(=O)=O)ccc3O[C@@H](C)C(F)(F)F)C4)no2)CC1 ; COC1(c2nc(C34CC3CN(C(=O)c3cc(S(C)(=O)=O)ccc3O[C@H](C)C(F)(F)F)C4)no2)CC1 ; C[C@@H](Oc1ccc(S(C)(=O)=O)cc1C(=O)N1CC2CC2(c2nnc(C(F)(F)F)o2)C1)C(F)(F)F]

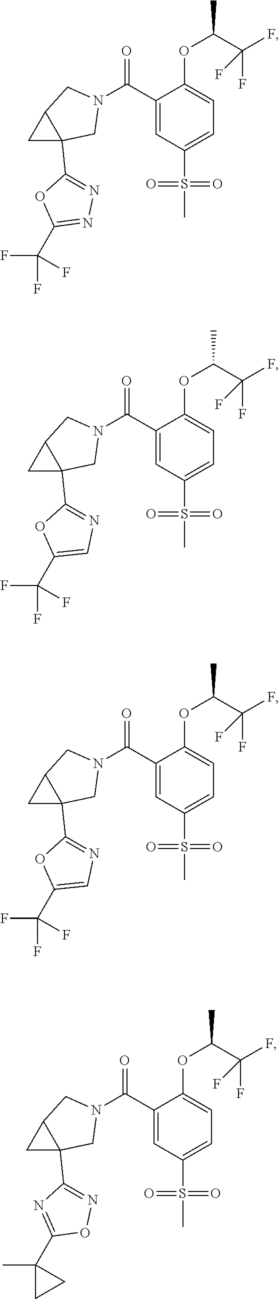 [SMILES: C[C@H](Oc1ccc(S(C)(=O)=O)cc1C(=O)N1CC2CC2(c2nnc(C(F)(F)F)o2)C1)C(F)(F)F ; C[C@@H](Oc1ccc(S(C)(=O)=O)cc1C(=O)N1CC2CC2(c2ncc(C(F)(F)F)o2)C1)C(F)(F)F ; C[C@H](Oc1ccc(S(C)(=O)=O)cc1C(=O)N1CC2CC2(c2ncc(C(F)(F)F)o2)C1)C(F)(F)F ; C[C@H](Oc1ccc(S(C)(=O)=O)cc1C(=O)N1CC2CC2(c2noc(C3(C)CC3)n2)C1)C(F)(F)F]

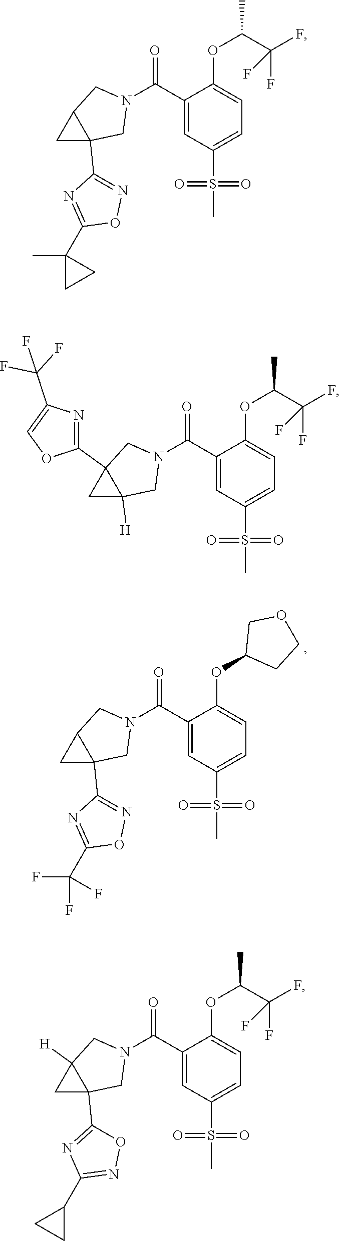 [SMILES: C[C@@H](Oc1ccc(S(C)(=O)=O)cc1C(=O)N1CC2CC2(c2noc(C3(C)CC3)n2)C1)C(F)(F)F ; C[C@H](Oc1ccc(S(C)(=O)=O)cc1C(=O)N1CC2CC2(c2nc(C(F)(F)F)co2)C1)C(F)(F)F ; CS(=O)(=O)c1ccc(O[C@@H]2CCOC2)c(C(=O)N2CC3CC3(c3noc(C(F)(F)F)n3)C2)c1 ; C[C@H](Oc1ccc(S(C)(=O)=O)cc1C(=O)N1CC2CC2(c2nc(C3CC3)no2)C1)C(F)(F)F]

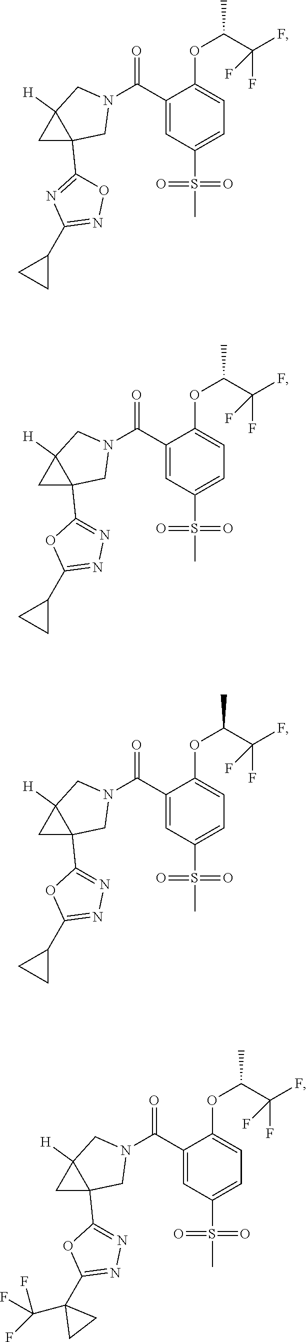 [SMILES: C[C@@H](Oc1ccc(S(C)(=O)=O)cc1C(=O)N1CC2CC2(c2nc(C3CC3)no2)C1)C(F)(F)F ; C[C@@H](Oc1ccc(S(C)(=O)=O)cc1C(=O)N1CC2CC2(c2nnc(C3CC3)o2)C1)C(F)(F)F ; C[C@H](Oc1ccc(S(C)(=O)=O)cc1C(=O)N1CC2CC2(c2nnc(C3CC3)o2)C1)C(F)(F)F ; C[C@@H](Oc1ccc(S(C)(=O)=O)cc1C(=O)N1CC2CC2(c2nnc(C3(C(F)(F)F)CC3)o2)C1)C(F)(F)F]

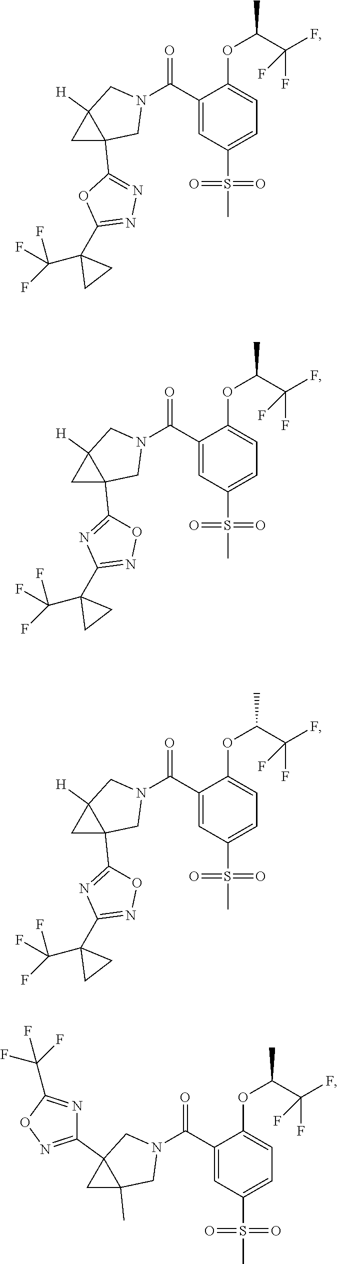 [SMILES: C[C@H](Oc1ccc(S(C)(=O)=O)cc1C(=O)N1CC2CC2(c2nnc(C3(C(F)(F)F)CC3)o2)C1)C(F)(F)F ; C[C@H](Oc1ccc(S(C)(=O)=O)cc1C(=O)N1CC2CC2(c2nc(C3(C(F)(F)F)CC3)no2)C1)C(F)(F)F ; C[C@@H](Oc1ccc(S(C)(=O)=O)cc1C(=O)N1CC2CC2(c2nc(C3(C(F)(F)F)CC3)no2)C1)C(F)(F)F ; C[C@H](Oc1ccc(S(C)(=O)=O)cc1C(=O)N1CC2(C)CC2(c2noc(C(F)(F)F)n2)C1)C(F)(F)F]

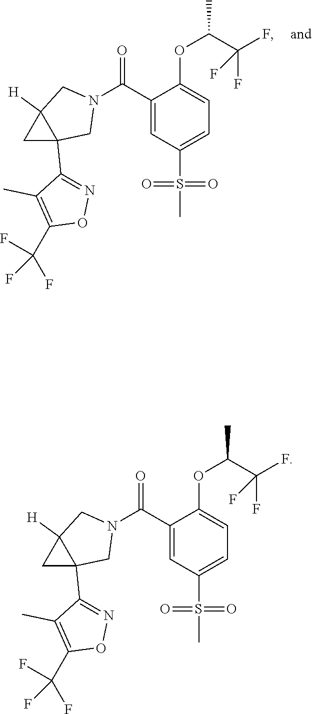 [SMILES: Cc1c(C23CC2CN(C(=O)c2cc(S(C)(=O)=O)ccc2O[C@H](C)C(F)(F)F)C3)noc1C(F)(F)F ; Cc1c(C23CC2CN(C(=O)c2cc(S(C)(=O)=O)ccc2O[C@@H](C)C(F)(F)F)C3)noc1C(F)(F)F]

In certain embodiments, the compound of Formula X is a compound having a formula

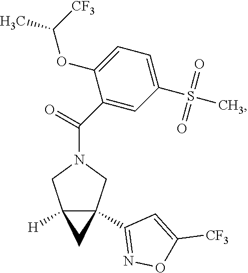 [SMILES: C[C@@H](Oc1ccc(S(C)(=O)=O)cc1C(=O)N1C[C@@H]2C[C@]2(c2cc(C(F)(F)F)on2)C1)C(F)(F)F]

or a pharmaceutically acceptable salt thereof.

In some embodiments of the methods and uses disclosed herein, the GlyT1 inhibitor is a compound of Formula XI,

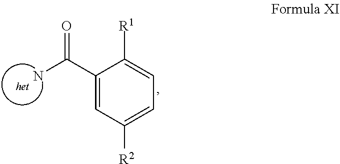

wherein:
R¹ is halogen, —OR^{1′}, —SR^{1′}, cycloalkyl, cyclic amide, heterocycloalkyl aryl or 5- or 6-membered heteroaryl containing one, two or three heteroatoms selected from the group consisting of oxygen, sulphur and nitrogen;
R^{1′} and R^{1″} are each independently hydrogen, lower alkyl, lower alkyl substituted by halogen —(CH₂)ₓ-cycloalkyl or —(CH₂)ₓ-aryl;
R² is —S(O)₂-lower alkyl, —S(O)₂NH-lower alkyl, NO₂ or CN;

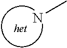

is an aromatic or partially aromatic bicyclic amine, having one or two additional N-atoms selected from the group consisting of

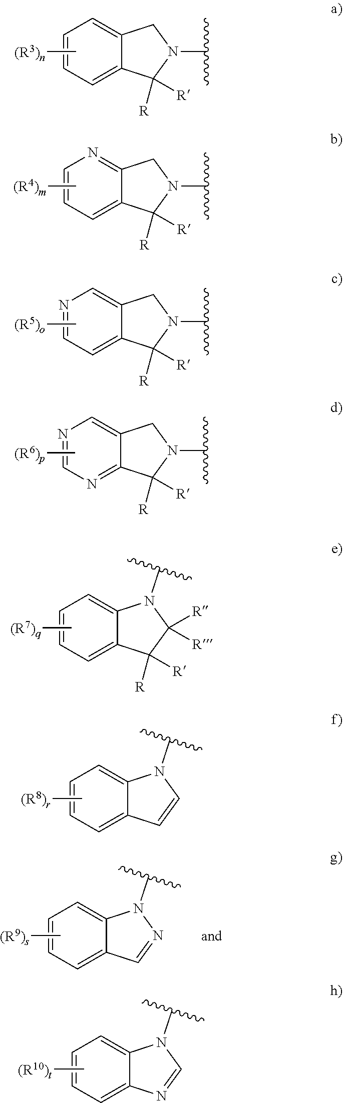

and wherein one of the additional N-ring atoms of the aromatic or partially aromatic bicyclic amine can be available in form of its oxide

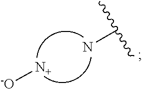

R3 to R10 are each independently hydrogen, hydroxy, halogen, ═O, lower allyl, cycloalkyl, heterocycloalkyl, lower alkoxy, CN, NO2, NH2, aryl, 5- or 6-membered heteroaryl containing one, two or three heteroatoms selected from the group consisting of oxygen, sulphur and nitrogen, —NH-lower alkyl, —N(lower alkyl)2, cyclic amide, —C(O)-cyclic amide, S-lower alkyl, —S(O)2-lower alkyl, lower alkyl substituted by halogen, lower alkoxy substituted by halogen, lower alkyl substituted by hydroxy, —O—(CH2)y-lower alkoxy, —O(CH2)yC(O)N(lower alkyl)2, —C(O)-lower alkyl, —O—(CH2)x-aryl, —O—(CH2)x-cycloalkyl, —O—(CH2)x-heterocycloalkyl, —C(O)O-lower alkyl, —C(O)—NH-lower alkyl, —C(O)—N(lower alkyl)2, 2-oxy-5-aza-bicyclo[2.2.1]hept-5-yl or 3-oxa-8-aza-bicyclo[3.2.1]oct-8-yl;
R, R′, R″ and R′″ are each independently hydrogen or lower alkyl; or
R′ and R′″ in group e) together with —(CH2)4- form a six membered ring;
and wherein all aryl-, cycloalkyl-, cyclic amide, heterocycloalkyl- or 5 or 6 membered heteroaryl groups as defined for R1, R1′, R1″ and R3 to R10 are unsubstituted or substituted by one or more substituents selected from the group consisting of hydroxy, ═O, halogen, lower allyl, phenyl, lower alkyl substituted by halogen and lower alkoxy;
n, m, o, p, q, r, s and t are each independently 1 or 2;
x is 0, 1 or 2; and
y is 1 or 2;
or a pharmaceutically acceptable salt thereof.

In certain embodiments, the compound of formula XI, or a pharmaceutically acceptable salt thereof, is a compound of formula XI(a),

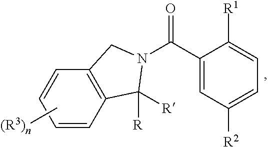

or a pharmaceutically acceptable salt thereof, a compound of formula XI(b),

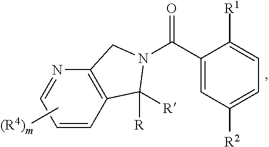

or a pharmaceutically acceptable salt thereof, a compound of formula XI(c),

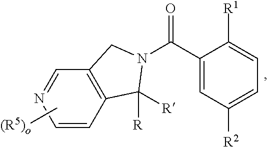

or a pharmaceutically acceptable salt thereof, a compound of formula XI(d),

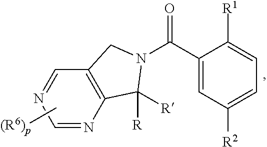

or a pharmaceutically acceptable salt thereof, a compound of formula XI(e),

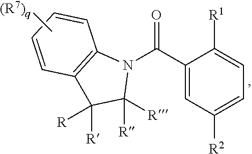

or a pharmaceutically acceptable salt thereof, a compound of formula XI(f),

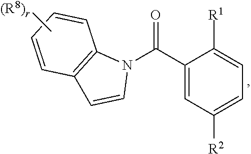

or a pharmaceutically acceptable salt thereof, a compound of formula XI(g),

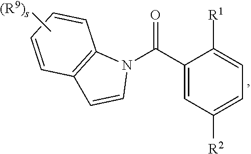

or a pharmaceutically acceptable salt thereof, or a compound of formula XI(h),

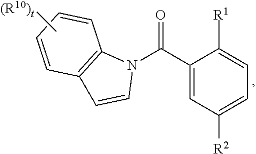

or a pharmaceutically acceptable salt thereof.

In certain embodiments, the compound of formula XI is a compound selected from any of the following, a stereoisomer or stereoisomeric mixture thereof, or a pharmaceutically acceptable salt thereof:

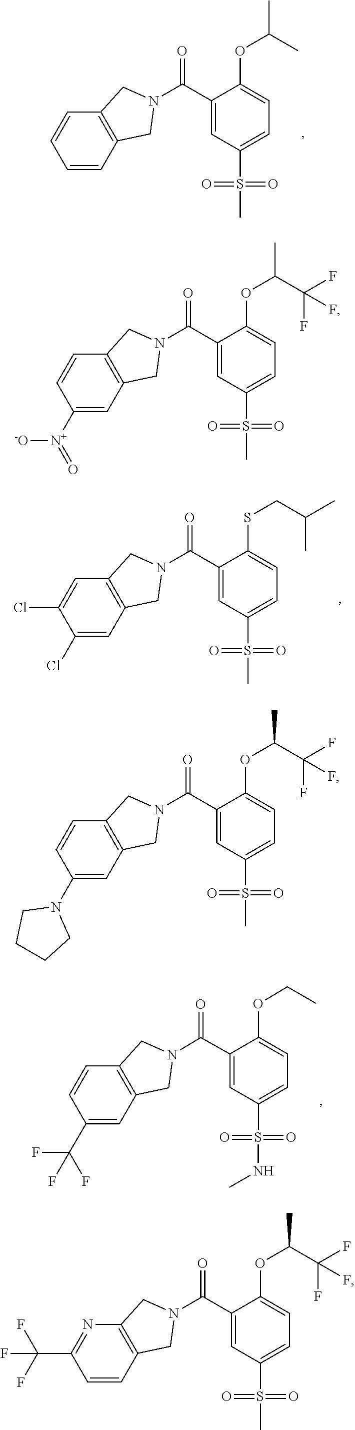

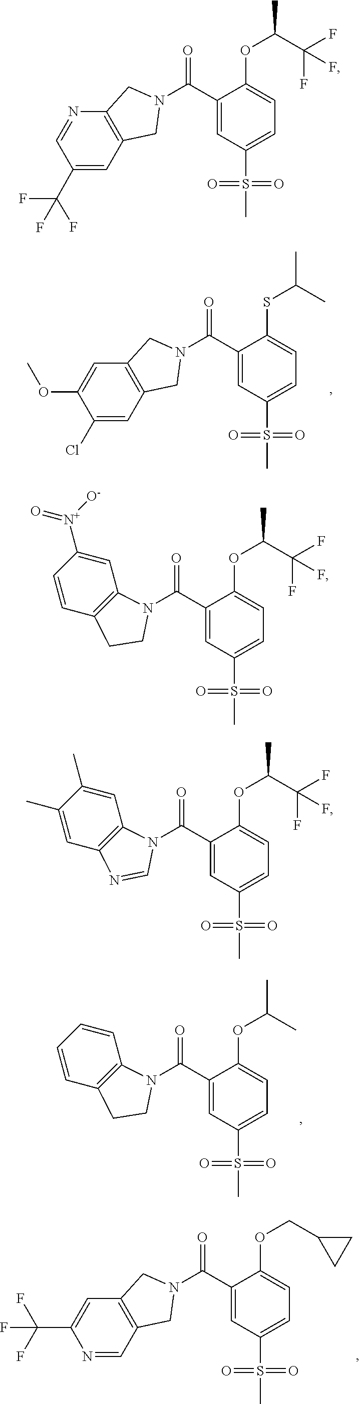

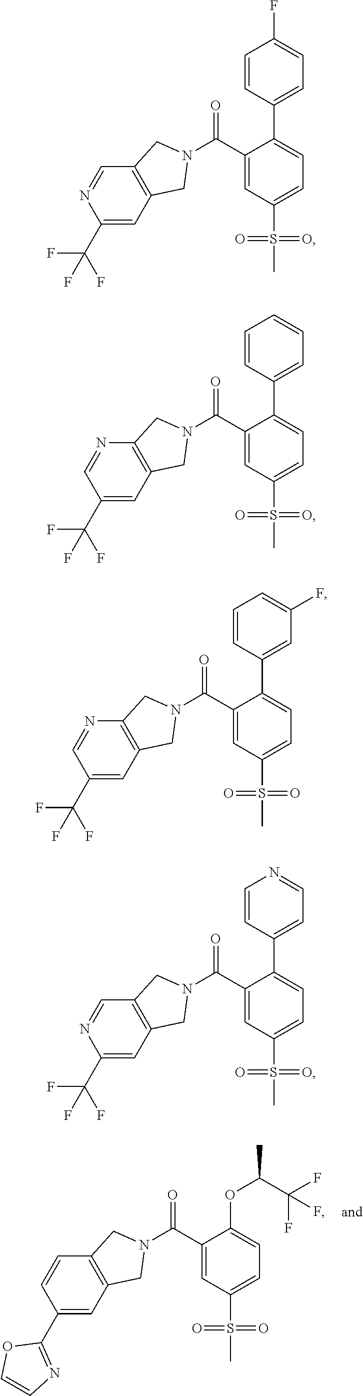

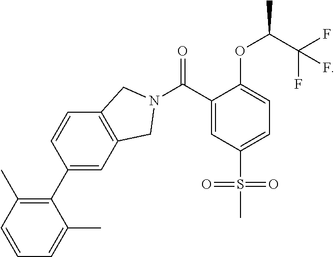 [SMILES: Cc1cccc(C)c1-c1ccc2c(c1)CN(C(=O)c1cc(S(C)(=O)=O)ccc1O[C@@H](C)C(F)(F)F)C2]

In certain of the methods and uses disclosed herein, the subject is a subject in need thereof.

In some embodiments of the uses and methods as disclosed herein, the glycine transporter inhibitor, such as a GlyT1 inhibitor (e.g., a GlyT1 inhibitor as disclosed herein), or a pharmaceutically acceptable salt thereof, or a prodrug of the glycine transporter inhibitor, such as a GlyT1 inhibitor (e.g., a GlyT1 inhibitor as disclosed herein), or its pharmaceutically acceptable salt is administered in a therapeutically effective amount.

In some embodiments, a compound, or a pharmaceutically acceptable salt, solvate or prodrug thereof, is chosen from a compound of as described herein. Any of the compounds provided for herein can be prepared as pharmaceutically acceptable salts, solvates or prodrugs and/or as part of a pharmaceutical composition as descripted in the cited patents or patent application publications herein.

Although the compounds described herein may be shown with specific stereochemistries around certain atoms, such as cis or trans, the compounds can also be made in the opposite orientation or in a racemic mixture. Such isomers or racemic mixtures are encompassed by the present disclosure. Additionally, although the compounds are shown collectively in a table, any compounds, or a pharmaceutically acceptable salt, solvate or prodrug thereof, can be chosen from the table and used in the embodiments provided for herein.

The compounds described herein can be made according to the methods described in the cited patents or patent application publications herein.

The compounds can be used to inhibit the GlyT1 transporter. Thus, in some embodiments, the compounds can be referred to as GlyT1 transporter inhibiting compounds or GlyT1 inhibitors.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginal, by inhalation, by depot injections, or by implants. The mode of administration can depend on the conditions or disease to be targeted or treated. The selection of the specific route of administration can be selected or adjusted by the clinician according to methods known to the clinician to obtain the desired clinical response.

In some embodiments, it may be desirable to administer one or more compounds, or a pharmaceutically acceptable salt, solvate or prodrug thereof, locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as silastic membranes, or fibers.

The compounds described herein can be administered either alone or in combination (concurrently or serially) with other pharmaceuticals. For example, the compounds can be administered in combination with other drugs for the treatment of a hepatic porphyria and the like. Examples of other pharmaceuticals or medicaments are known to one of skill in the art and include, but are not limited to those described herein.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above. The selection of the specific dose regimen can be selected or adjusted or titrated by the clinician according to methods known to the clinician to obtain the desired clinical response.

The amount of a compound described herein that will be effective in the treatment and/or prevention of a particular disease, condition, or disorder will depend on the nature and extent of the disease, condition, or disorder, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about 10 mg to about 35 mg per kg body weight. Suitable dosage ranges for other modes of administration can be calculated based on the forgoing dosages as known by those skilled in the art. For example, recommended dosages for intranasal, transmucosal, intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

In certain embodiments, the glycine transporter inhibitor to be administered is a GlyT1 inhibitor, such as a GlyT1 inhibitor as disclosed herein. In some embodiments, suitable dosage ranges for the GlyT1 inhibitor are from about 5 mg/day to 200 mg/day. In some embodiments, the GlyT1 inhibitor is administered at 5 mg/day. In some embodiments, the GlyT1 inhibitor is administered at 10 mg/day. In some embodiments, the GlyT1 inhibitor is administered at 15 mg/day. In some embodiments, the GlyT1 inhibitor is administered at 20 mg/day. In some embodiments, the GlyT1 inhibitor is administered at 25 mg/day. In some embodiments, the GlyT1 inhibitor is administered at 30 mg/day. In some embodiments, the GlyT1 inhibitor is administered at 35 mg/day. In some embodiments, the GlyT1 inhibitor is administered at 40 mg/day. In some embodiments, the GlyT1 inhibitor is administered at 45 mg/day. In some embodiments, the GlyT1 inhibitor is administered at 50 mg/day. In some embodiments, the GlyT1 inhibitor is administered at 55 mg/day. In some embodiments, the GlyT1 inhibitor is administered at 60 mg/day. In some embodiments, the GlyT1 inhibitor is administered at 65 mg/day. In some embodiments, the GlyT1 inhibitor is administered at 70 mg/day. In some embodiments, the GlyT1 inhibitor is administered at 75 mg/day. In some embodiments, the GlyT1 inhibitor is administered at 80 mg/day. In some embodiments, the GlyT1 inhibitor is administered at 85 mg/day. In some embodiments, the GlyT1 inhibitor is administered at 90 mg/day. In some embodiments, the GlyT1 inhibitor is administered at 95 mg/day. In some embodiments, the GlyT1 inhibitor is administered at 100 mg/day. In some embodiments, the GlyT1 inhibitor is administered at 105 mg/day. In some embodiments, the GlyT1 inhibitor is administered at 110 mg/day. In some embodiments, the GlyT1 inhibitor is administered at 115 mg/day. In some embodiments, the GlyT1 inhibitor is administered at 120 mg/day. In some embodiments, the GlyT1 inhibitor is administered at 125 mg/day. In some embodiments, the GlyT1 inhibitor is administered at 130 mg/day. In some embodiments, the GlyT1 inhibitor is administered at 135 mg/day. In some embodiments, the GlyT1 inhibitor is administered at 140 mg/day. In some embodiments, the GlyT1 inhibitor is administered at 145 mg/day. In some embodiments, the GlyT1 inhibitor is administered at 150 mg/day. In some embodiments, the GlyT1 inhibitor is administered at 155 mg/day. In some embodiments, the GlyT1 inhibitor is administered at 160 mg/day. In some embodiments, the GlyT1 inhibitor is administered at 165 mg/day. In some embodiments, the GlyT1 inhibitor is administered at 170 mg/day. In some embodiments, the GlyT1 inhibitor is administered at 175 mg/day. In some embodiments, the GlyT1 inhibitor is administered at 180 mg/day. In some embodiments, the GlyT1 inhibitor is administered at 185 mg/day. In some embodiments, the GlyT1 inhibitor is administered at 190 mg/day. In some embodiments, the GlyT1 inhibitor is administered at 195 mg/day. In some embodiments, the GlyT1 inhibitor is administered at 200 mg/day.

In certain embodiments, the glycine transporter inhibitor to be administered is a GlyT1 inhibitor, such as bitopertin, pharmaceutically acceptable salt thereof, or a prodrug of bitopertin or its pharmaceutically acceptable salt. In some embodiments, the GlyT1 inhibitor is bitopertin. In some embodiments, suitable dosage ranges for bitopertin are from about 5 mg/day to 200 mg/day. In some embodiments, bitopertin is administered at 5 mg/day. In some embodiments, bitopertin is administered at 10 mg/day. In some embodiments, bitopertin is administered at 15 mg/day. In some embodiments, bitopertin is administered at 20 mg/day. In some embodiments, bitopertin is administered at 25 mg/day. In some embodiments, bitopertin is administered at 30 mg/day. In some embodiments, bitopertin is administered at 35 mg/day. In some embodiments, bitopertin is administered at 40 mg/day. In some embodiments, bitopertin is administered at 45 mg/day. In some embodiments, bitopertin is administered at 50 mg/day. In some embodiments, bitopertin is administered at 55 mg/day. In some embodiments, bitopertin is administered at 60 mg/day. In some embodiments, bitopertin is administered at 65 mg/day. In some embodiments, bitopertin is administered at 70 mg/day. In some embodiments, bitopertin is administered at 75 mg/day. In some embodiments, bitopertin is administered at 80 mg/day. In some embodiments, bitopertin is administered at 85 mg/day. In some embodiments, bitopertin is administered at 90 mg/day. In some embodiments, bitopertin is administered at 95 mg/day. In some embodiments, bitopertin is administered at 100 mg/day. In some embodiments, bitopertin is administered at 105 mg/day. In some embodiments, bitopertin is administered at 110 mg/day. In some embodiments, bitopertin is administered at 115 mg/day. In some embodiments, bitopertin is administered at 120 mg/day. In some embodiments, bitopertin is administered at 125 mg/day. In some embodiments, bitopertin is administered at 130 mg/day. In some embodiments, bitopertin is administered at 135 mg/day. In some embodiments, bitopertin is administered at 140 mg/day. In some embodiments, bitopertin is administered at 145 mg/day. In some embodiments, bitopertin is administered at 150 mg/day. In some embodiments, bitopertin is administered at 155 mg/day. In some embodiments, bitopertin is administered at 160 mg/day. In some embodiments, bitopertin is administered at 165 mg/day. In some embodiments, bitopertin is administered at 170 mg/day. In some embodiments, bitopertin is administered at 175 mg/day. In some embodiments, bitopertin is administered at 180 mg/day. In some embodiments, bitopertin is administered at 185 mg/day. In some embodiments, bitopertin is administered at 190 mg/day. In some embodiments, bitopertin is administered at 195 mg/day. In some embodiments, bitopertin is administered at 200 mg/day.

The compounds described herein can be formulated for parenteral administration by injection, such as by bolus injection or continuous infusion. In some embodiments, the compounds can be administered by continuous infusion subcutaneously over a period of about 15 minutes to about 24 hours. Formulations for injection can be presented in unit dosage form, such as in ampoules or in multi-dose containers, with an optionally added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides. The compounds described herein can also be formulated in vaginal compositions such as vaginal creams, suppositories, pessaries, vaginal rings, and intrauterine devices.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

It is also known in the art that the compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts, solvates or prodrugs thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I-VIII is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A.R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of compounds include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can ophthalmically acceptable glycols, polyglycols, e.g., polyethylene glycol 400 (PEG-400), and glycol ethers.

Suitable solubilizing agents for solution and solution/suspension compositions are cyclodextrins. Suitable cyclodextrins can be chosen from α-cyclodextrin, β-cyclodextrin, γ-cyclodextrin, allylcyclodextrins (e.g., methyl-β-cyclodextrin, dimethyl-β-cyclodextrin, diethyl-β-cyclodextrin), hydroxyallylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed in Rajewski et al., Journal of Pharmaceutical Sciences, 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts, solvates or prodrugs can be included in the compositions in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally one or more acceptable surfactants, such as, but not limited to, nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In some embodiments, pharmaceutical packs or kits comprising one or more containers filled with one or more compounds described herein are provided. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one compound described herein. In some embodiments, the kit comprises a compound described herein in a single injectable dosage form, such as a single dose within an injectable device such as a syringe with a needle.

In some embodiments, the methods comprise administering to the subject one or more compounds described herein or a pharmaceutically acceptable salt, solvate or prodrug thereof, or a pharmaceutical composition of the same. In some embodiments, the subject is a subject in need of such treatment. As described herein, in some embodiments, the subject is a mammal, such as, but not limited to, a human.

In some embodiments, also provided are one or more compounds described above, or a pharmaceutically acceptable salt, solvate or prodrug thereof, or a pharmaceutical composition comprising one or more compounds described above, for use in the manufacture of a medicament for the treatment of methods of treating and/or preventing a hepatic porphyria, or related syndrome thereof, including, but not limited to the conditions described herein, in a subject, such as those described herein. In some embodiments, the subject is a subject in need thereof.

The present embodiments also provides the use of one or more compounds described above, or a pharmaceutically acceptable salt, solvate or prodrug thereof, or a pharmaceutical composition comprising one or more compounds described above, in the inhibition of a GlyT1 transporter, such as the presence on the surface of the cell. In some embodiments, the compounds, pharmaceutically acceptable salt thereof, or a pharmaceutical composition of the same inhibit the internalization, trafficking, and/or degradation of the GlyT1 transporter.

As used herein, “inhibition” can refer to either inhibition of a specific activity. The activity of a GlyT1 transporter can be measured by any method known in the art including but not limited to the methods described herein.

The compounds described herein are inhibitors of the GlyT1 transporter. The ability of the compounds to inhibit GlyT1 transporter activity may be measured using any assay known in the art.

Generally, assays for testing compounds that inhibit GlyT1 transporter activity include the determination of any parameter that is indirectly or directly under the influence of a GlyT1 transporter, e.g., a functional, physical, or chemical effect.

Samples or assays comprising GlyT1 transporters that are treated with a potential inhibitor, are compared to control samples without the inhibitor to examine the extent of inhibition. Control samples (untreated with inhibitors) are assigned a relative GlyT1 transporter activity value of 100%. Inhibition of a GlyT1 transporter is achieved when the GlyT1 transporter activity value relative to the control is about 80%, 50%, or 25%.

Ligand binding to a GlyT1 transporter can be tested in a number of formats. Binding can be performed in solution, in a bilayer membrane, attached to a solid phase, in a lipid monolayer, or in vesicles. For example, in an assay, the binding of the natural ligand to its transporter is measured in the presence of a candidate modulator, such as the compound described herein. Alternatively, the binding of the candidate modulator may be measured in the presence of the natural ligand. Often, competitive assays that measure the ability of a compound to compete with binding of the natural ligand to the transporter are used. Binding can be tested by measuring, e.g., changes in spectroscopic characteristics (e.g., fluorescence, absorbance, refractive index), hydrodynamic (e.g., shape) changes, or changes in chromatographic or solubility properties.

After the transporter is expressed in cells, the cells can be grown in appropriate media in the appropriate cell plate. The cells can be plated, for example at 5000-10000 cells per well in a 384 well plate. In some embodiments, the cells are plated at about 1000, 2000, 3000, 4000, 5000, 6000, 7000, 8000, 9000, or 10000 cells/per well. The plates can have any number of wells and the number of cells can be modified accordingly.

Any medicament having utility in an application described herein can be used in co-therapy, co-administration or co-formulation with a composition as described above. Therefore, the compounds described herein can be administered either before, concurrently with, or after such therapeutics are administered to a subject.

The additional medicament can be administered in co-therapy (including co-formulation) with the one or more of the compounds described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 1 to about 24, about 2 to about 12 hours, from about 3 to about 8 hours, or from about 4 to about 6 hours. In some embodiments, the dose is administered 1, 2, 3, or 4 times a day. It will be understood by those of skill in the art that an appropriate dosing interval is dependent to some degree on the length of time for which the selected composition is capable of maintaining a concentration of the compound(s) in the subject and/or in the target tissue (e.g., above the EC₅₀ (the minimum concentration of the compound which inhibits the transporter's activity by 90%). Ideally the concentration remains above the EC₅₀ for at least 100% of the dosing interval. Where this is not achievable it is desired that the concentration should remain above the EC₅₀ for at least about 60% of the dosing interval or should remain above the EC₅₀ for at least about 40% of the dosing interval.

### Methods of Use

The present application provides methods of preventing or treating a hepatic porphyria in a subject, the method comprising administering to the subject one or more glycine transporter inhibitor or a pharmaceutically acceptable salt thereof, or a prodrug of the one or more glycine transporter inhibitor or its pharmaceutically acceptable salt. In certain embodiments, the glycine transporter inhibitor is a GlyT1 inhibitor, such as a GlyT1 inhibitor as disclosed herein. For example, the present application provides a method of preventing, treating, or reducing the progression rate and/or severity of a hepatic porphyria in a subject, comprising administering to the subject bitopertin,

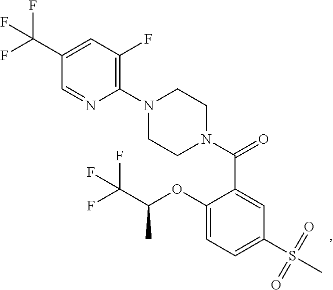 [SMILES: C[C@H](Oc1ccc(S(C)(=O)=O)cc1C(=O)N1CCN(c2ncc(C(F)(F)F)cc2F)CC1)C(F)(F)F]

or a pharmaceutically acceptable salt thereof, or a prodrug of bitopertin or its pharmaceutically acceptable salt.

In part, the present disclosure relates to methods of treating a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the disclosure relates to methods of preventing, treating, or reducing the progression rate and/or severity of one or more complications of a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt.

In some embodiments, the hepatic porphyria is acute hepatic porphyria. In some embodiments, the hepatic porphyria is non-acute hepatic porphyria. In some embodiments, the hepatic porphyria is acute intermittent porphyria (AIP). In some embodiments, the hepatic porphyria is ALA dehydratase porphyria (ADP). In some embodiments, the hepatic porphyria is variegate porphyria (VP). In some embodiments, the hepatic porphyria is hereditary coproporphyria (HCP). In some embodiments, the hepatic porphyria is harderoporphyria. In some embodiments, the hepatic porphyria is porphyria cutanea tarda (PCT). In some embodiments, the PCT is familial or sporadic PCT. In some embodiments, the hepatic porphyria is hepatoerythropoietic porphyria (HEP).

The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The present application provides methods of preventing, treating, or reducing the progression rate and/or severity of a hepatic porphyria, the method comprising administering to the subject one or more glycine transporter inhibitor, or a pharmaceutically acceptable salt thereof, or a prodrug of the one or more glycine transporter inhibitor or its pharmaceutically acceptable salt. In some embodiments, the one or more glycine transporter inhibitor is one or more GlyT1 and/or GlyT2 inhibitors. In some embodiments, the one or more glycine transporter inhibitor is one or more GlyT1 inhibitors, such as one or more GlyT1 inhibitors as disclosed herein. In certain embodiments of the foregoing, the pharmaceutical composition further comprises a pharmaceutically acceptable carrier. For example, the present application provides a method of preventing, treating, or reducing the progression rate and/or severity of a hepatic porphyria in a subject, comprising administering to the subject bitopertin, or a pharmaceutically acceptable salt thereof, or a prodrug of bitopertin or its pharmaceutically acceptable salt.

The present application further provides use of one or more glycine transporter inhibitor, or a pharmaceutically acceptable salt thereof, or a prodrug of the one or more glycine transporter inhibitor or its pharmaceutically acceptable salt, in the manufacture of a formulation for the treatment of a hepatic porphyria in a subject. In some embodiments, the one or more glycine transporter inhibitor is one or more GlyT1 and/or GlyT2 inhibitors. In some embodiments, the one or more glycine transporter inhibitor is one or more GlyT1 inhibitor, such as one or more GlyT1 inhibitor as disclosed herein. In certain such embodiments, the GlyT1 inhibitor is bitopertin, or a pharmaceutically acceptable salt thereof, or a prodrug of bitopertin or its pharmaceutically acceptable salt. In certain embodiments of the foregoing, the formulation is administered in a therapeutically effective amount.

### Hepatic Porphyrias

Porphyrias comprise eight inherited metabolic disorders of heme biosynthesis in which various enzymes in the complex heme biosynthetic pathway are disrupted. Porphyrias are broadly classified as acute vs non-acute or hepatic vs erythropoietic porphyrias, based on their clinical presentation. Acute hepatic porphyrias include acute intermittent porphyria (AIP), variegate porphyria (VP), hereditary coproporphyria (HCP), and aminolevulinic acid dehydratase deficient porphyria (ADP), and often lead to serious abdominal, psychiatric, neurologic, or cardiovascular symptoms. AIP, HCP, and VP are autosomal dominant porphyrias and ADP is autosomal recessive porphyria. In rare cases, AIP, HCP, and VP occur as homozygous dominant forms. Porphyria cutanea tarda (PCT) is a non-acute hepatic porphyria in which patients often present with blisters, bullae, milia, and hypertrichosis on cheeks, temples, and eyebrows. In addition, there is a rare homozygous recessive form of PCT known as hepatoerythropoietic porphyria (HEP). The clinical and laboratory features of these porphyrias are described in Table 1 below.

TABLE 1
Symptoms and Diagnostic Strategy in Hepatic Porphyrias and Lead Poisoning

| | Enzyme | | Diagnosis in symptomatic patients | | | |
|---|---|---|---|---|---|---|
| Porphyria | Activity | Symptoms | Urine | Stool | Erythrocytes | Plasma |

Acute Hepatic Porphyrias

| | | | | | | |
|---|---|---|---|---|---|---|
| ADP | ALAD↓ | NV, A¹ | ALA, Copro | — | Zn-Proto | — |
| | | | III | | IX | |
| AIP | PBGD↓ | NV, LD¹ | ALA, PBG, | — | — | 615-620 |
| | | | Uro III | | | |
| HCP | CPOX↓ | NV, C¹, | ALA, PBG, | Copro III | — | 615-620 |
| | | LD¹ | Copro III | | | |
| VP | PPOX↓ | NV, C¹, | ALA, PBG, | Proto > | — | 624-627 |
| | | LD¹ | CoproIII | Copro | | |

Non-Acute Hepatic Porphyrias

| | | | | | | |
|---|---|---|---|---|---|---|
| PCT and | UROD↓ | C, A¹, | Uro III, | Isocopro, | — | 615-620 |
| HEP | | LD | Hepta | Hepta | | |

Other

| | | | | | | |
|---|---|---|---|---|---|---|
| Lead | ALAD↓ | NV, A¹, | ALA, PBG, | — | Zn-Proto | 615-620 |
| Poisoning | | LD¹ | Copro III | | IX | |

NV: neurovisceral symptoms;
C: cutaneous symptoms;
A: Anemia;
LD: liver damage;
¹clinical feature variable at time of manifestation;
ALA: 5-aminolevulinic acid;
Copro: coproporphyrin;
PBG: porphobilinogen;
Uro: uroporphyrin;
Hepta: heptacarboxyl-porphyrin;
Proto: protoporphyrin;
Isocopro: isocoproporphyrin;

Porphyrias are a family of inherited or acquired disorders resulting from the deficient activity of specific enzymes in the heme biosynthetic pathway, also referred to herein as the porphyrin pathway. Porphyrins are the main precursors of heme. Porphyrins and porphyrin precursors include 5-aminolevulinic acid (ALA), porphopilinogen (PBG), hydroxymethylbilane (HMB), uroporphyrinogen I or III, coproporphyrinogen I or III, protoporphrinogen IX, and protoporphyrin IX. Heme is an essential part of hemoglobin, myoglobin, catalases, peroxidases, and cytochromes, the latter including the respiratory and P450 liver cytochromes. Heme is synthesized in most or all human cells. About 85% of heme is made in erythroid cells, primarily for hemoglobin. Most of the remaining heme is made in the liver, 80% of which is used for the synthesis of cytochromes. Deficiency of specific enzymes in the porphyrin pathway leads to insufficient heme production and also to an accumulation of porphyrin precursors and/or porphyrins, which can be toxic to cell or organ function in high concentrations.

Porphyrias may be classified by the primary site of the overproduction and accumulation of porphyrins or their precursors. In hepatic porphyrias, porphyrins and porphyrin precursors are overproduced predominantly in the liver, whereas in erythropoietic porphyrias, porphyrins are overproduced in the erythroid cells in the bone. The acute or hepatic porphyrias lead to dysfunction of the nervous system and neurologic manifestations that can affect both the central and peripheral nervous system, resulting in symptoms such as, for example, pain (e.g., abdominal pain and/or chronic neuropathic pain), vomiting, neuropathy (e.g., acute neuropathy progressive neuropathy), muscle weakness, seizures, mental disturbances (e.g., hallucinations, depression anxiety, paranoia), cardiac arrhythmias, tachycardia, constipation, and diarrhea. The cutaneous or erythropoietic porphyrias primarily affect the skin, causing symptoms such as photosensitivity that can be painful, blisters, necrosis, itching, swelling, and increased hair growth on areas such as the forehead. Subsequent infection of skin lesions can lead to bone and tissue loss, as well as scarring, disfigurement, and loss of digits (e.g., fingers, toes). Most porphyrias are caused by mutations that encode enzymes in the heme biosynthetic pathway.

Not all porphyrias are genetic. For example, patients with liver disease may develop porphyria as a result of liver dysfunction. Patients with PCT can acquire the deficient activity of uroporphyrinogen decarboxylase (URO-D), due to the formation of a ORO-D enzyme with lower than normal enzymatic activity.

Acute intermittent porphyria (AIP) (also be referred to as porphobilinogen (PBG) deaminase deficiency, or hydroxymethylbilane synthase (HMBS) deficiency), is the most common type of acute hepatic porphyria. Other types of acute hepatic porphyrias include hereditary coproporphyria (HCP), variegate porphyria (VP), and ALA deyhdratase deficiency porphyria (ADP). Non-acute hepatic porphyrias include porphyria cutanea tarda (PCT), a disease in which patients often present with blisters, bullae, milia, and hypertrichosis on cheeks, temples, and eyebrows. In addition, there is a rare homozygous recessive form of PCT known as hepatoerythropoietic porphyria (HEP). The clinical and laboratory features of these porphyrias are described in Table 1.

AIP has been found to have a prevalence as high as 1 in 10,000 in certain populations (e.g., in Northern Sweden). The prevalence of mutations in the general population in United States and Europe, excluding the U.K., is estimated to be about 1 in 10,000 to 1 in 20,000. Clinical disease manifests itself in only approximately 10-15% of individuals who carry mutations that are known to be associated with AIP. However, the penetrance is as high as 40% in individuals with certain mutations (e.g., the W198X mutation). AIP is typically latent prior to puberty. Symptoms are more common in females than in males. The prevalence of the disease is probably underestimated due to its incomplete penetrance and long periods of latency. In the United States, it is estimated that there are about 2000 patients who have suffered at least one attack. It is estimated that there are about 150 active recurrent cases in France, Sweden, the U.K., and Poland; these patients are predominantly young women, with a median age of 30.

AIP affects, for example, the visceral, peripheral, autonomic, and central nervous systems. Symptoms of AIP are variable and include gastrointestinal symptoms (e.g., severe and poorly localized abdominal pain, nausea/vomiting, constipation, diarrhea, ileus), urinary symptoms (dysuria, urinary retention/incontinence, or dark urine), neurologic symptoms (e.g., sensory neuropathy, motor neuropathy (e.g., affecting the cranial nerves and/or leading to weakness in the arms or legs), seizures, neuropathic pain (e.g., pain associated with progressive neuropathy, e.g., chronic neuropathic pain), neuropsychiatric symptoms (e.g., mental confusion, anxiety, agitation, hallucination, hysteria, delirium, apathy, depression, phobias, psychosis, insomnia, somnolence, coma), autonomic nervous system involvement (resulting e.g., in cardiovascular symptoms such as tachycardia, hypertension, and/or arrhythmias, as well as other symptoms, such as, e.g., increased circulating catecholamine levels, sweating, restlessness, and/or tremor), dehydration, and electrolyte abnormalities. The most common symptoms are abdominal pain and tachycardia. In addition, patients frequently have chronic neuropathic pain and develop a progressive neuropathy. Patients with recurring attacks often have a prodrome. Permanent paralysis may occur after a severe attack. Recovery from severe attacks that are not promptly treated may take weeks or months. An acute attack may be fatal, for example, due to paralysis of respiratory muscles or cardiovascular failure from electrolyte imbalance. Prior to the availability of Hemin treatments, up to 20% of patients with AIP died from the disease.

In individuals who carry genes for AIP, the risk of hepatocellular cancer is increased. In those with recurrent attacks, the risk of hepatocellular cancer is particularly grave: after the age of 50, the risk is nearly 100-fold greater than in the general population.

Attacks of acute porphyria may be precipitated by endogenous or exogenous factors. The mechanisms by which such factors induce attacks may include, for example, increased demand for hepatic P450 enzymes and/or induction of ALAS1 activity in the liver. Increased demand for hepatic P450 enzymes results in decreased hepatic free heme, thereby inducing the synthesis of hepatic ALAS1.

Precipitating factors include fasting (or other forms of reduced or inadequate caloric intake, due to crash diets, long-distance athletics, etc.), metabolic stresses (e.g., infections, surgery, international air travel, and psychological stress), endogenous hormones (e.g., progesterone), cigarette smoking, lipid-soluble foreign chemicals (including, e.g., chemicals present in tobacco smoke, certain prescription drugs, organic solvents, biocides, components in alcoholic beverages), endocrine factors (e.g., reproductive hormones (women may experience exacerbations during the premenstrual period), synthetic estrogens, progesterones, ovulation stimulants, and hormone replacement therapy).

Over 1000 drugs are contraindicated in the acute hepatic porphyrias (e.g., AIP, HCP, ADP, and VP) including, for example, alcohol, barbiturates, Carbamazepine, Carisoprodol, Clonazepam (high doses), Danazol, Diclofenac and possibly other NSAIDS, Ergots, estrogens, Ethyclorvynol, Glutethimide, Griseofulvin, Mephenytoin, Meprobamate (also mebutamate and tybutamate), Methyprylon, Metodopramide, Phenytoin, Primidone, progesterone and synthetic progestins, Pyrazinamide, Pyrazolones (aminopyrine and antipyrine), Rifampin, Succinimides (ethosuximide and methsuximide), sulfonamide antibiotics, and Valproic acid.

Objective signs of AIP include discoloration of the urine during an acute attack (the urine may appear red or red-brown), and increased concentrations of PBG and ALA in urine during an acute attack. Molecular genetic testing identifies mutations in the PBG deaminase (also known as HMBS) gene in more than 98% of affected individuals.

The differential diagnosis of porphyrias may involve determining the type of porphyria by measuring individual levels of porphyrins or porphyrin precursors (e.g., ALA, PBG) in the urine, feces, and/or plasma (e.g., by chromatography and fluorometry) during an attack. The diagnosis of AIP can be confirmed by establishing that erythrocyte PBG deaminase activity is at 50% or less of the normal level. DNA testing for mutations may be carried out in patients and at-risk family members. The diagnosis of AIP is typically confirmed by DNA testing to identify a specific causative gene mutation (e.g., an HMBS mutation).

Treatment of acute attacks typically requires hospitalization to control and treat acute symptoms, including, e.g., abdominal pain, seizures, dehydration/hyponatremia, nausea/vomiting, tachycardia/hypertension, urinary retention/ileus. For example, abdominal pain may be treated, e.g., with narcotic analgesics, seizures may be treated with seizure precautions and possibly medications (although many anti-seizure medications are contraindicated), nausea/vomiting may be treated, e.g., with phenothiazines, and tachycardia/hypertension may be treated, e.g., with beta blockers. Treatment may include withdrawal of unsafe medications, monitoring of respiratory function, as well as muscle strength and neurological status. Mild attacks (e.g., those with no paresis or hyponatremia) may be treated with at least 300 g intravenous 10% glucose per day, although increasingly hemin is provided immediately. Severe attacks should be treated as soon as possible with intravenous hemin (3-4 mg/kg daily for 4-14 days) and with IV glucose while waiting for the IV hemin to take effect. Typically, attacks are treated with IV hemin for 4 days and with IV glucose while waiting for administration of the IV hemin.

Hemin (Panhematin® or hemin for injection, previously known as hematin) is the only heme product approved for use in the United States and was the first drug approved under the Orphan Drug Act. Panhematin® is hemin derived from processed red blood cells (PRBCs), and is Protoporphyrin IX containing a ferric iron ion (Heme B) with a chloride ligand. Heme acts to limit the hepatic and/or marrow synthesis of porphyrin. The exact mechanism by which hemin produces symptomatic improvement in patients with acute episodes of the hepatic porphyrias has not been elucidated; however, its action is likely due to the (feedback) inhibition of 5-aminolevulinic acid (ALA) synthase, the enzyme which limits the rate of the porphyrin/heme biosynthetic pathway. Inhibition of ALA synthase should result in reduced production of ALA and PBG as well as porphyrins and porphyrin intermediates.

Drawbacks of hemin include its delayed impact on clinical symptoms and its failure to prevent the recurrence of attacks. Adverse reactions associated with hemin administration may include thrombophlebitis, anticoagulation, thrombocytopenia, renal shut down, or iron overload, which is particularly likely in patients requiring multiple courses of hemin treatment for recurrent attacks. To prevent phlebitis, an indwelling venous catheter is needed for access in patients with recurrent attacks. Uncommonly reported side effects include fever, aching, malaise, hemolysis, anaphalaxis, and circulatory collapse.

Heme is difficult to prepare in a stable form for intravenous administration. It is insoluble at neutral pH but can be prepared as heme hydroxide at pH 8 or higher. Panhematin® is a lyophilized hemin preparation. When lyophilized hemin is solubilized for intravenous administration, degradation products form rapidly; these degradation products are responsible for a transient anticoagulant effect and for phlebitis at the site of infusion. Heme albumin and heme arginate (Normosang, the European version of hemin) are more stable and may potentially cause less thrombophlebitis. However, heme arginate is not approved for use in the United States. Panhemin® may be stabilized by solubilizing it for infusion in 30% human albumin rather than in sterile water; however, albumin adds intravascular volume-expanding effects and increases the cost of treatment as well as risk of pathogens since it is isolated from human blood.

The successful treatment of an acute attack does not prevent or delay recurrence. There is a question of whether hemin itself can trigger recurring attacks due to induction of heme oxygenase. Nonetheless, in some areas (especially France), young women with multiply recurrent attacks are being treated with weekly hemin with the goal of achieving prophylaxis.

Givosiran (Givlaari®), an aminolevulinate synthase 1-directed small interfering ribonucleic acid (siRNA) is also used to treat patients with acute hepatic porphyrias by targeting and degrading ALAS1 mRNA in hepatocytes using RNA interference. The concerned risks associated with the use of givosiran include anaphylactic reactions, liver toxicity, and renal toxicity. For example, 15% patients in givosiran clinical trials showed transaminase (ALT) elevations 3 times the upper limit of normal. Additionally, 15% of patients receiving givosiran have renal-related adverse reactions including elevated serum creatinine levels and decreased estimated glomerular filtration rate.

Limited experience with liver transplantation suggests that if successful, it is an effective treatment for AIP. There have been approximately 12 transplants in Europe in human patients, with curative or varying effects. Liver transplantation can restore normal excretion of ALA and PBG and prevent acute attacks. Furthermore, if the liver of a patient with AP is transplanted into another patient (“domino transplant”), the patient receiving the transplant may develop AIP. While orthotrophic liver transplantation is curative, this procedure has significant morbidity and mortality and the availability of liver donors is limited.

Among the long-term clinical effects of acute porphyrias is chronic neuropathic pain that may result from a progressive neuropathy due to neurotoxic effects, e.g., of elevated porphyrin precursors (e.g., ALA and/or PBG). Patients may suffer from neuropathic pain prior to or during an acute attack. Older patients may experience increased neuropathic pain with age for which various narcotic drugs are typically prescribed. Electromyogram abnormalities and decreased conduction times have been documented in patients with acute hepatic porphyrias. In patients with acute porphyria (e.g., ADP, AIP, HCP, or VP), levels of porphyrin precursors (ALA & PBG) are often elevated in asymptomatic patients and in symptomatic patients between attacks. Thus, reduction of the porphyrin precursors and resumption of normal heme biosynthesis by reducing the level of ALAS1 expression and/or activity is expected to prevent and/or minimize development of chronic and progressive neuropathy. Treatment, e.g., chronic treatment (e.g., periodic treatment with iRNA as described herein, e.g., treatment according to a dosing regimen as described herein, e.g., weekly or biweekly treatment) can continuously reduce the ALAS1 expression in acute porphyria patients who have elevated levels of porphyrin precursors, porphyrins, porphyrin products or their metabolites. Such treatment may be provided as needed to prevent or reduce the frequency or severity of an individual patient's symptoms (e.g., pain and/or neuropathy) and/or to reduce a level of a porphyrin precursor, porphyrin, porphyrin product or metabolite.

The need exists for identifying novel therapeutics that can be used for the treatment of porphyrias. As discussed above, existing treatments such as hemin, givosiran, and liver transplant have numerous drawbacks. For example, the impact of hemin on clinical symptoms is delayed, it is expensive, and it may have side effects (e.g., thrombophlebitis, anticoagulation, thrombocytopenia, iron overload, renal shutdown).

### Treatment of Hepatic Porphyrias

In some embodiments, the disclosure provides methods of preventing or treating a hepatic porphyria in a subject, the method comprising administering to the subject one or more glycine transporter inhibitor or a pharmaceutically acceptable salt thereof, or a prodrug of the one or more glycine transporter inhibitor or its pharmaceutically acceptable salt. In certain embodiments, the glycine transporter inhibitor is a GlyT1 inhibitor, such as a GlyT1 inhibitor as disclosed herein.

In some embodiments, the subject has or is at risk for developing a hepatic porphyria (e.g., AIP, HCP, VP, ADP, PCT, and HEP). In some embodiments, the hepatic porphyria is an acute hepatic porphyria (e.g., AIP, HCP, VP, and ADP). In some embodiments, the hepatic porphyria is a non-acute hepatic porphyria (e.g., PCT and HEP). In some embodiments, the hepatic porphyria is a dual hepatic porphyria, e.g., at least two hepatic porphyrias. In some embodiments, the dual hepatic porphyria comprises two or more hepatic porphyrias selected from the group consisting of AIP, HCP, VP, ADP, PCT, and HEP.

In some embodiments, the hepatic porphyria is a caused by a heterozygous mutation resulting in reduced enzymatic activity. In some embodiments, the hepatic porphyria is a caused by a homozygous mutation resulting in reduced enzymatic activity. In some embodiments, the hepatic porphyria is an autosomal recessive diseases (e.g., ADP). In some embodiments, the subject carries a genetic alteration (e.g., a mutation) as described herein but is otherwise asymptomatic.

A mutation associated with a hepatic porphyria includes mutations in a gene encoding certain enzymes in the heme biosynthetic pathway (porphyrin pathway) or a gene which alters the expression of a gene in the heme biosynthetic pathway (e.g., ALAD, HMBS, UROD, UROS, CPOX, and PPOX). In many embodiments, the subject carries one or more mutations in an enzyme of the porphyrin pathway (e.g., ALA-dehydratase, PBG deaminase, uroporphyrinogen III synthase, uroporphyrinogen III synthase, uroporphyrinogen decarboxylase, coproporphyrinogen oxidase, and protoporphyrinogen oxidase).

In some embodiments, patients with an acute hepatic porphyria (e.g., AIP), or patients who carry mutations associated with an acute hepatic porphyria (e.g., AIP) but who are asymptomatic, have elevated ALA and/or PBG levels compared with healthy individuals. In such cases, the level of ALA and/or PBG can be elevated even when the patient is not having, or has never had, an attack. In some such cases, the patient is otherwise completely asymptomatic. In some such cases, the patient suffers from pain, e.g., neuropathic pain, which can be chronic pain (e.g., chronic neuropathic pain). In some cases, the patient has a neuropathy. In some cases, the patient has a progressive neuropathy.

In some embodiments, the subject has an acute attack of hepatic porphyria. In some embodiments, the subject has a non-acute attack of hepatic porphyria. In some embodiments, the subject has never experienced an acute attack of hepatic porphyria. In some embodiments, the subject suffers from chronic pain. In some embodiments, the subject has nerve damage. In some embodiments, the subject has EMG changes and/or changes in nerve conduction velocity. In some embodiments, the subject is asymptomatic. In some embodiments, the subject is at risk for developing a hepatic porphyria (e.g., carries a gene mutation associated with a hepatic porphyria) and is asymptomatic. In some embodiments, the subject has previously had an acute attack of hepatic porphyria but is asymptomatic at the time of treatment.

In some embodiments, the subject is at risk for developing a hepatic porphyria and is treated prophylactically to prevent the development of a hepatic porphyria. In some embodiments the subject has an elevated level of a porphyrin or a porphyrin precursor (e.g., ALA and/or PBG). In some embodiments, the prophylactic treatment begins at puberty. In some embodiments the treatment lowers the level (e.g., the plasma level or the urine level) of a porphyrin or a porphyrin precursor (e.g., ALA and/or PBG). In some embodiments, the treatment prevents the development of an elevated level of a porphyrin or a porphyrin precursor, (e.g., ALA and/or PBG). In some embodiments, the treatment prevents the development of, or decreases the frequency or severity of, a symptom associated with a hepatic porphyria (e.g., pain or nerve damage).

In some embodiments, the subject to be treated according to the methods described suffers from pain, e.g., chronic pain. In some embodiments, the method is effective to treat the pain (e.g., by reducing the severity of the pain or curing the pain). In some embodiments, the method is effective to decrease or prevent nerve damage.

In some embodiments, the subject to be treated according to the methods described herein (a) has an elevated level of ALA and/or PBG and (b) suffers from pain (e.g., chronic pain). In some embodiments, the method is effective to decrease an elevated level of ALA and/or PBG and/or to treat the pain (e.g., by reducing the severity of the pain or curing the pain).

In some embodiments, the subject is a subject who has suffered one or more acute attacks of one or more hepatic porphyric symptoms. In other embodiments, the subject is a subject who has suffered chronically from one or more symptoms of hepatic porphyria (e.g., pain, e.g., neuropathic pain and or neuropathy, e.g., progressive neuropathy). In some embodiments, the subject to be treated according to the methods described herein has recently experienced or is currently experiencing a prodrome.

A “prodrome,” as used herein, includes any symptom that the individual subject has previously experienced immediately prior to developing an acute attack. Typical symptoms of a prodrome include, e.g., abdominal pain, nausea, headaches, psychological symptoms (e.g., anxiety), restlessness and/or insomnia. In some embodiments, the subject experiences pain (e.g., abdominal pain and/or a headache) during the prodrome. In some embodiments, the subject experiences nausea during the prodrome. In some embodiments, the subject experiences psychological symptoms (e.g., anxiety) during the prodrome. In some embodiments, the subject becomes restless and/or suffers from insomnia during the prodrome.

An acute “attack” of hepatic porphyria involves the onset of one or more symptoms of hepatic porphyria, typically in a patient who carries a mutation associated with hepatic porphyria (e.g., a mutation in a gene that encodes an enzyme in the porphyrin pathway).

In some embodiments, the GlyT1 inhibitor is administered after an acute attack of a hepatic porphyria. In some embodiments, the GlyT1 inhibitor is administered during an acute attack of a hepatic porphyria. In some embodiments, administration of a GlyT1 inhibitor is effective to lessen the severity of the attack (e.g., by ameliorating one or more signs or symptoms associated with the attack). In some embodiments, administration of a GlyT1 inhibitor is effective to shorten the duration of an attack. In some embodiments, administration of an a GlyT1 inhibitor is effective to stop an attack. In some embodiments, the GlyT1 inhibitor is administered prophylactically to prevent an acute attack of hepatic porphyria. In some embodiments, the prophylactic administration is before, during, or after exposure to or occurrence of a precipitating factor. In some embodiments, the subject is at risk of developing porphyria.

A “precipitating factor” as used herein, refers to an endogenous or exogenous factor that may induce an acute attack of one or more symptoms associated with porphyria. Precipitating factors include fasting (or other forms of reduced or inadequate caloric intake, due to crash diets, long-distance athletics, etc.), metabolic stresses (e.g., infections, surgery, international air travel, and psychological stress), endogenous hormones (e.g., progesterone), cigarette smoking, lipid-soluble foreign chemicals (including, e.g., chemicals present in tobacco smoke, certain prescription drugs, organic solvents, biocides, components in alcoholic beverages), endocrine factors (e.g., reproductive hormones (women may experience exacerbations during the premenstrual period), synthetic estrogens, progesterones, ovulation stimulants, and hormone replacement therapy), and lead. Other common precipitating factors include cytochrome P450 inducing drugs and phenobarbitol.

In some embodiments, the GlyT1 inhibitor is administered during a prodrome. In some embodiments, the prodrome is characterized by pain (e.g., headache and/or abdominal pain), nausea, psychological symptoms (e.g., anxiety), restlessness and/or insomnia. In some embodiments, the GlyT1 inhibitor is administered during a particular phase of the menstrual cycle, e.g., during the luteal phase.

In some embodiments, administration of a GlyT1 inhibitor is effective to prevent attacks (e.g., recurrent attacks that are associated with a prodrome and/or with a precipitating factor, e.g., with a particular phase of the menstrual cycle, e.g., the luteal phase). In some embodiments, administration of an GlyT1 inhibitor is effective to reduce the frequency of attacks. In some embodiments, administration of a GlyT1 inhibitor is effective to lessen the severity of the attack (e.g., by ameliorating one or more signs or symptoms associated with the attack). In some embodiments, administration of a GlyT1 inhibitor is effective to shorten the duration of an attack. In some embodiments, administration of a GlyT1 inhibitor is effective to stop an attack.

In some embodiments administration of a GlyT1 inhibitor is effective to prevent or decrease the frequency or severity of pain, e.g., neuropathic pain. In some embodiments administration of a GlyT1 inhibitor is effective to prevent or decrease the frequency or severity of neuropathy. In some embodiments, the subject has or is at risk for developing a hepatic porphyria and suffers from pain (e.g., neuropathic pain, e.g., chronic neuropathic pain) or neuropathy (e.g., progressive neuropathy). In some embodiments, the subject has an elevated level of ALA and/or PBG and suffers from chronic pain.

Effects of administration of a GlyT1 inhibitor can be established, for example, by comparison with an appropriate control. For example, a decrease in the frequency of acute attacks, as well as a decrease in the level of one or more porphyrins or porphyrin precursors, may be established, for example, in a group of patients with AIP, as a decreased frequency compared with an appropriate control group. A control group (e.g., a group of similar individuals or the same group of individuals in a crossover design) may include, for example, an untreated population, a population that has been treated with a conventional treatment for hepatic porphyria (e.g., a conventional treatment for AIP may include glucose, hemin, or both); a population that has been treated with placebo, or a GlyT1 inhibitor, optionally in combination with one or more conventional treatments for hepatic porphyria (e.g., glucose, e.g., IV glucose), and the like.

A subject “at risk” of developing hepatic porphyria, as used herein, includes a subject with a family history of hepatic porphyria and/or a history of one or more recurring or chronic hepatic porphyria symptoms, and/or a subject who carries a genetic alteration (e.g., a mutation) in a gene encoding an enzyme of the heme biosynthetic pathway, and a subject who carries a genetic alteration, e.g., a mutation known to be associated with hepatic porphyria.

In some embodiments, the alteration, e.g., the mutation, makes an individual susceptible to an acute attack (e.g., upon exposure to a precipitating factor, e.g., a drug, dieting or other precipitating factor, e.g., a precipitating factor as disclosed herein). In some embodiments, the alteration, e.g., the mutation, is associated with elevated levels of a porphyrin or a porphyrin precursor (e.g., ALA and/or PBG). In some embodiments, the alteration, e.g., the mutation, is associated with chronic pain (e.g., chronic neuropathic pain) and/or neuropathy (e.g., progressive neuropathy). In some embodiments, the alteration, e.g., the mutation, is associated with changes in EMG and/or nerve conduction velocities.

In some embodiments, the alteration is a mutation in a gene selected from the group consisting of ALAD, HMBS, UROD, CPOX, and PPOX. In some embodiments, the alteration is an alteration, e.g., a mutation, in a gene that encodes an enzyme in the heme biosynthetic pathway. In some embodiments, the subject has a genetic alteration but does not suffer from acute attacks. In some embodiments, the subject has a mutation associated with AIP, HCP, VP, ADP, PCT, or HEP.

In some embodiments, the hepatic porphyria is AIP. In some such embodiments, the subject has an alteration, e.g., at least one mutation, in PBGD (gene encoding PBG deaminase). Many PBGD mutations are known in the art. In some embodiments, the subject is heterozygous for a PBGD mutation. In other embodiments, the subject is homozygous for a PBGD mutation. A homozygous subject may carry two identical mutations or two different mutations in the PBGD gene. In some embodiments, the hepatic porphyria is HCP. In some embodiments, the subject has an alteration, e.g., at least one mutation, in CPOX (i.e, gene that encodes the enzyme coproporphyrinogen III oxidase). In some embodiments, the hepatic porphyria is VP. In some embodiments, the subject has an alteration, e.g., at least one mutation, in PPOX (i.e., gene that encodes protoporphrinogen oxidase). In some embodiments, the hepatic porphyria is ADP (e.g., autosomal recessive ADP). In some embodiments, the subject has an alteration, e.g., at least one mutation, in ALAD (gene that encodes ALA dehydratase). In some embodiments, the hepatic porphyria is PCT. In some embodiments, the subject has an alteration, e.g., at least one mutation, in UROD (gene that encodes uro-decarboxylase). In some embodiments, the hepatic porphyria is CEP. In some embodiments, the subject has an alteration, e.g., at least one mutation, in UROS (gene that encodes uroporphyrinogen III synthase).

In some embodiments, the increased levels of porphyrin precursors is due to lead poisoning. Lead poisoning inhibits the activity of each of ALAD, CPOX, and FECH, enzymes which are involved in heme biosynthesis. Patients with lead poisoning are frequently misdiagnosed with ADP or other acute porphyrias. In some embodiments, a subject with lead poisoning has decreased enzymatic activity of ALAD. In some embodiments, a subject with lead poisoning has decreased enzymatic activity of CPOX. In some embodiments, a subject with lead poisoning has decreased enzymatic activity of FECH. In some embodiments, a subject with lead poisoning has increased levels of lead in the blood and/or urine. In some embodiments, a subject with lead poisoning has increased levels of ALA. In some embodiments, a subject with lead poisoning has increased levels of ALA and PBG. In some embodiments, a subject with lead poisoning has ALA levels which are increased by at least 10 fold over a reference value. In some embodiments, a subject with lead poisoning has ALA levels which are increased by at least 5 fold over a reference value. In some embodiments, the disclosure relates to methods of treating lead poisoning in a subject, comprising administering to a subject a GlyT1 inhibitor, or a pharmaceutically acceptable salt thereof, or a prodrug of the GlyT1 inhibitor or its pharmaceutically acceptable salt. In some embodiments, the subject is further administered a chelating agent. In some embodiments the chelating agent is 2,3-dimercaptosuccinic acid. In some embodiments, the chelating agent is calcium disodium ethylenediamine-tetraacetate.

Porphyrins (e.g., 5-ALA, PBG, uroporphyrin, and coproporphyrin) can be found in various biological samples including the skin, urine, stool, plasma, and erythrocytes. In some embodiments, the porphyrins may be extracted from the biological sample (e.g., plasma) into a solution for fluorescence analysis. Porphyrins can be detected in these biological samples by direct inspection using long wavelength ultraviolet light (e.g., 400-420 nm light). Porphyrins have the greatest absorption wavelengths near 400-420 nm, with their highest absorption peak occurring at 415 nm. The emission maxima of porphyrins is typically around 600 nm and varies slightly based on the type of porphyrins and the solvent used for analysis. In some embodiments, diagnosis of a hepatic porphyria may be made using fluorescence analysis. In some embodiments, skin porphyrin levels can be measured by calculating the difference before and after complete photobleaching of the skin porphyrin using controlled illumination. See, e.g., Heerfordt I M. Br J Dermatol. 2016; 175(6):1284-1289.

In some embodiments, the subject's plasma porphyrin fluoresces at a peak of 634 nm when illuminated with blue light (e.g., 400-420 nm light). In some embodiments, the subject's plasma porphyrin fluoresces at a peak between 626 nm and 634 nm when illuminated with blue light (e.g., 400-420 nm light). In some embodiments, the subject's skin porphyrin fluoresces at a peak of 632 nm when illuminated with blue light (e.g., 400-420 nm light). In some embodiments, the subject's skin porphyrin fluoresces at a peak between 626 nm and 634 nm when illuminated with blue light (e.g., 400-420 nm light). In some embodiments, a sample from the subject (e.g., plasma or skin) containing a porphyrin or porphyrin precursor fluoresces at a peak between 615 nm and 620 nm when illuminated with blue light (e.g., 400-420 nm light). In some embodiments, a sample from the subject (e.g., plasma or skin) containing a porphyrin or porphyrin precursor fluoresces at a peak between 624 nm and 627 nm when illuminated with blue light (e.g., 400-420 nm light). In some embodiments, the subject's plasma is excited using a 405 nm laser. In some embodiments, the subject has red fluorescent urine.

### Heme and Heme Intermediates

Glycine is one of the key initial substrates for heme and globin synthesis. As such, decreased levels of glycine due to GlyT1 inhibition could lead to a decrease in heme synthesis. In certain aspects, the disclosure relates to methods of treating a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt, wherein the subject's heme levels decrease no more than 10% (e.g., 10%, 15%, 20%, 25%, and 30%). In some embodiments, the disclosure relates to methods of treating a hepatic porphyria in a subject, wherein the subject's heme levels decrease no more than 15%. In some embodiments, the disclosure relates to methods of treating a hepatic porphyria in a subject, wherein the subject's heme levels decrease no more than 20%. In some embodiments, the disclosure relates to methods of treating a hepatic porphyria in a subject, wherein the subject's heme levels decrease no more than 25%. In some embodiments, the disclosure relates to methods of treating a hepatic porphyria in a subject, wherein the subject's heme levels decrease no more than 30%. In certain aspects, the disclosure relates to methods of treating a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt, wherein the dosage of the pharmaceutical composition does not cause a substantial reduction in heme levels.

In some embodiments, the synthesis of one or more of the following heme intermediates (e.g., porphyrin precursors) is inhibited, wherein the one or more heme intermediates is selected from the group consisting of 5-ALA, PBG, hydroxymethylbilane, ZPPIX, uroporphyrinogen I, uroporphyrinogen III, heptacarboxyporphyrinogen I, heptacarboxyporphyrinogen III, hexacarboxyporphyrinogen I, hexacarboxyporphyrinogen III, pentacarboxyporphyrinogen I, pentacarboxyporphyrinogen III, coproporphyrinogen I, coproporphyrinogen III, isocoproporphyrin, porphobilinogen; and protoporphyrinogen IX. In some embodiments, the disclosure relates to methods of inhibiting 5-aminolevulinic acid (5-ALA) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has a hepatic porphyria. In some embodiments, the disclosure relates to methods of inhibiting coproporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting zinc-protoporphyrin IX (ZPPIX) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has ALA dehydratase porphyria (ADP). In some embodiments, the disclosure relates to methods of inhibiting porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting 5-aminolevulinic acid (5-ALA) and porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting hydroxymethylbilane (HMB) synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting uroporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting heptacarboxyl-porphyrin synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting isocoproporphyrin synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the synthesis of the one or more heme intermediates (e.g., 5-ALA, coproporphyrin III, ZPPIX, PBG, HMB, uroporphyrin III, heptacarboxyl-porphyrin, and isocoproporphyrin) is inhibited in a dose dependent manner.

In some embodiments, the accumulation of one or more of the following heme intermediates (e.g., porphyrin precursors) is inhibited, wherein the one or more heme intermediates is selected from the group consisting of 5-ALA, PBG, hydroxymethylbilane, ZPPIX, uroporphyrinogen I, uroporphyrinogen III, heptacarboxyporphyrinogen I, heptacarboxyporphyrinogen III, hexacarboxyporphyrinogen I, hexacarboxyporphyrinogen III, pentacarboxyporphyrinogen I, pentacarboxyporphyrinogen III, coproporphyrinogen I, coproporphyrinogen III, isocoproporphyrin, porphobilinogen; and protoporphyrinogen IX. In some embodiments, the accumulation of the one or more heme intermediates (e.g., 5-ALA, coproporphyrin III, ZPPIX, PBG, HMB, uroporphyrin III, heptacarboxyl-porphyrin, and isocoproporphyrin) is inhibited in a dose dependent manner.

In some embodiments, the subject to be treated according to the methods described herein has an elevated level of a porphyrin or a porphyrin precursor, e.g., ALA and/or PBG. In some embodiments, the subject has porphyrin precursor level that is at least 10%, 20%, 30%, 40%, or 50% more than porphyrin precursor level in a healthy subject prior to administration of the GlyT1 inhibitor. In some embodiments, the subject has increased levels of a porphyrin precursor. In some embodiments, the porphyrin precursor is selected from the group consisting of 5-ALA, HMB, coproporphyrin III, ZPPIX, porphobilinogen, uroporphyrin III, heptacarboxyl-porphyrin, and isocoproporphyrin. In some embodiments, the subject has increased uroporphyrin III levels (e.g., increased uroporphyrin III levels in the urine). In some embodiments, the subject has increased levels of 5-ALA (e.g., increased levels of 5-ALA in the urine or plasma). In some embodiments, the subject has increased levels of HMB. In some embodiments, the subject has increased levels of coproporphyrin III (e.g., increased levels of coproporphyrin III in the urine and stool). In some embodiments, the subject has increased levels of PBG (e.g., increased levels of PBG in the urine). In some embodiments, the subject has an increased proportion of protoporphyrin to coproporphyrin in the stool. In some embodiments, the subject has increased heptacarboxyl-porphyrin levels (e.g., increased heptacarboxyl-porphyrin levels in the urine or stool). In some embodiments, the subject has increased isocoproporphyrin levels (e.g., increased isocoproporphyrin levels in the stool). In some embodiments, the subject has increased ZPPIX levels in erythrocytes.

Levels of a porphyrin or a porphyrin precursor can be assessed using methods known in the art or methods described herein. In some embodiments, the level of a porphyrin or a porphyrin precursor (e.g., ALA or PBG) in the subject is assessed based on the absolute level of the porphyrin or the porphyrin precursor, e.g., ALA or PBG in a sample from the subject. In some embodiments, the level of a porphyrin or a porphyrin precursor (e.g., ALA or PBG) in the subject is assessed based on the relative level of the porphyrin or porphyrin precursor (e.g., ALA or PBG) in a sample from the subject. In some embodiments, the relative level is relative to the level of another protein or compound, e.g., the level of creatinine, in a sample from the subject. In some embodiments, the sample is a urine sample. In some embodiments, the sample is a plasma sample. In some embodiments, the sample is a stool sample.

An elevated level of a porphyrin or a porphyrin precursor (e.g., ALA and/or PBG) can be established by showing that the subject has a level of a porphyrin or a porphyrin precursor (e.g., a plasma or urine level of ALA and/or PBG) that is greater than, or greater than or equal to, a reference value. A physician with expertise in the treatment of porphyrias would be able to determine whether the level of a porphyrin or a porphyrin precursor (e.g., ALA and/or PBG) is elevated, e.g., for the purpose of diagnosing a hepatic porphyria or for determining whether a subject is at risk for developing a hepatic porphyria, e.g., a subject may be predisposed to an acute attack or to pathology associated with a porphyria, such as, e.g., chronic pain (e.g., neuropathic pain) and neuropathy (e.g., progressive neuropathy).

As used herein, a “reference value” refers to a value from the subject when the subject is not in a disease state, or a value from a normal or healthy subject, or a value from a reference sample or population, e.g., a group of normal or healthy subjects (e.g., a group of subjects that does not carry a mutation associated with a hepatic porphyria and/or a group of subjects that does not suffer from symptoms associated with a hepatic porphyria).

In some embodiments, the reference value is a pre-disease level in the same individual. In some embodiments, the reference value is a level in a reference sample or population. In some embodiments, the reference value is the mean or median value in a reference sample or population. In some embodiments, the reference value the value that is two standard deviations above the mean in a reference sample or population. In some embodiments, the reference value is the value that is 2.5, 3, 3.5, 4, 4.5, or 5 standard deviations above the mean in a reference sample or population.

In some embodiments, the subject has a plasma level or a urine level of ALA or PBG that is greater than a reference value. In some embodiments, wherein the subject has an elevated level of a porphyrin or a porphyrin precursor (e.g., ALA and/or PBG) the subject has a level of ALA and/or PBG that is at least 10%, 20%, 30%, 40%, 50%, 60%, 70%, 80%, or 90% higher than a reference value. In some embodiments, the subject has a level of a porphyrin or a porphyrin precursor (e.g., ALA and/or PBG) that is at least 2, 3, 4, 5, 6, 7, 8, 9, or 10 fold higher than a reference value.

In some embodiments, the reference value is an upper reference limit. As used herein, an “upper reference limit” refers to a level that is the upper limit of the 95% confidence interval for a reference sample or population, e.g., a group of normal (e.g., wild type) or healthy individuals, e.g., individuals who do not carry a genetic mutation associated with a porphyria and/or individuals who do not suffer from a hepatic porphyria. Accordingly, a lower reference limit refers to a level that is the lower limit of the same 95% confidence interval.

In some embodiments, the subject has an elevated level (e.g., a plasma level or a urine level) of a porphyrin or a porphyrin precursor that is greater than or equal to 2 times, 3 times, 4 times, or 5 times that of a reference value (e.g., an upper reference limit). In some embodiments, the subject has a urine level of a porphyrin or a porphyrin precursor that is greater than 4 times that of an upper reference limit.

In some embodiments, the subject has a urine level of PBG that is greater than or equal to 1.4 mmol/mol creatinine. In some embodiments, the subject has a urine level of PBG that is greater than or equal to 4.8 mmol/mol creatinine. In certain embodiments, the subject has a urine level of PBG that is greater than, or greater than or equal to, about 3, 4, 5, 6, 7, or 8 mmol/mol creatinine.

In some embodiments, the reference value for plasma PBG is 0.12 μmol/L. In some embodiments, the subject has a plasma PBG level that is greater than, or greater than or equal to 0.10 μmol/L, 0.12 μmol/L, 0.24 μmol/L, 0.36 μmol/L, 0.48 μmol/L, or 0.60 μmol/L. In some embodiments, the subject has a plasma level of PBG that is greater than, or greater than or equal to 0.48 μmol/L.

In some embodiments, the reference value for urine PBG is 1.2 mmol/mol creatinine. In some embodiments, the reference value for urine PBG is 1.4 mmol/mol creatinine. In some embodiments, the subject has a urine PBG level that is greater than, or greater than or equal to 1.0 mmol/mol creatinine, 1.2 mmol/mol creatinine, 2.4 mmol/mol creatinine, 3.6 mmol/mol creatinine, 4.8 mmol/mol creatinine, or 6.0 mmol/mol creatinine. In some embodiments, the subject has a urine level of PBG that is greater than, or greater than or equal to 4.8 mmol/mol creatinine.

In some embodiments, the reference value for plasma ALA is 0.12 μmol/L. In some embodiments, the subject has a plasma ALA level that is greater than, or greater than or equal to 0.10 μmol/L, 0.12 μmol/L, 0.24 μmol/L, 0.36 μmol/L, 0.48 μmol/L, or 0.60 μmol/L. In some embodiments, the subject has a plasma ALA level that is greater than, or greater than or equal to 0.48 μmol/L.

In some embodiments, the reference value for urine ALA is 3.1 mmol/mol creatinine. In some embodiments, the reference value for urine ALA is 6.3 mmol/mol creatinine. In some embodiments, the subject has a urine ALA level that is greater than, or greater than or equal to 2.5 mmol/mol creatinine, 3.1 mmol/mol creatinine, 6.2 mmol/mol creatinine, 6.3 mmol/mol creatinine, 9.3 mmol/mol creatinine, 12.4 mmol/mol creatinine, or 15.5 mmol/mol creatinine.

In some embodiments, the reference value for urine uroporphyrin is less than 4.5 μmol/mol creatinine. In some embodiments, the subject has a urine uroporphyrin level that is greater than, or greater than or equal to 4.5 μmol/mol creatinine, 9.0 μmol/mol creatinine, 13.5 μmol/mol creatinine, 18.0 μmol/mol creatinine, 22.5 μmol/mol creatinine, 27 μmol/mol creatinine, or 31.5 μmol/mol creatinine.

In some embodiments, the reference value for urine coproporphyrin is less than 20.7 μmol/mol creatinine. In some embodiments, the subject has a urine coproporphyrin level that is greater than, or greater than or equal to 20.7 μmol/mol creatinine, 41.4 μmol/mol creatinine, 62.1 μmol/mol creatinine, 82.8 μmol/mol creatinine, 103.5 μmol/mol creatinine, 124.2 μmol/mol creatinine, or 144.9 μmol/mol creatinine.

In some embodiments, the reference value for plasma porphyrin is 10 nmol/L. In some embodiments, the subject has a plasma porphyrin level that is greater than, or greater than or equal to 10 nmol/L. In some embodiments, the subject has a plasma porphyrin level that is greater than, or greater than or equal to 8, 10, 15, 20, 25, 30, 35, 40, 45, or 50 nmol/L. In some embodiments, the subject has a plasma porphyrin level that is greater than, or greater than or equal to 40 nmol/L.

In some embodiments, the reference value for urine porphyrin is 25 μmol/mol creatinine. In some embodiments, the reference value for urine porphyrin is less than 28.4 μmol/mol creatinine. In some embodiments, the subject has a urine porphyrin level that is greater than, or greater than or equal to 25 μmol/mol creatinine. In some embodiments, the subject has a urine porphyrin level that is greater than, or equal to 20, 25, 30, 35, 40, 45, 50, 55, 60, 65, 70, 75, or 80 μmol/mol creatinine.

In some embodiments, the subject has a level (e.g., a plasma level or a urine level) of a porphyrin or a porphyrin precursor that is greater than that of 99% of individuals in a sample of healthy individuals.

In some embodiments, the subject has a level (e.g., a plasma level or a urine level) of ALA or PBG that is greater than two standard deviations above the mean level in a sample of healthy individuals.

In some embodiments, the subject has a urine level of ALA that is 1.6 or more times that of the mean level in a normal subject (e.g., a subject that does not carry a mutation associated with a porphyria). In some embodiments, the subject has a plasma level of ALA that is 2 or 3 times that of the mean level in a normal subject. In some embodiments, the subject has a urine level of PBG that is four or more times that of the mean level in a normal subject. In some embodiments, the subject has a plasma level of PBG that is four or more times that of the mean level in a normal subject.

In certain embodiments, administration of a GlyT1 inhibitor results in a decrease in the level of one or more porphyrins or porphyrin precursors, as described herein (e.g., ALA and/or PBG). The decrease may be measured relative to any appropriate control or reference value. For example, the decrease in the level of one or more porphyrins or porphyrin precursors may be established in an individual subject, e.g., as a decrease of at least 5%, 10%, 15%, 20%, 25%, 30%, 35%, 40%, 45%, 50% or more compared with the level prior to treatment (e.g., immediately prior to treatment). A decrease in the level of a porphyrin precursor, a porphyrin, or a porphyrin metabolite may be measured using any method known in the art.

In some embodiments, administration of a GlyT1 inhibitor is effective to reduce the level of ALA and/or PBG in the subject. The level of ALA or PBG in the subject can be assessed, e.g., based on the absolute level of ALA or PBG, or based on the relative level of ALA or PBG (e.g., relative to the level of another protein or compound, e.g., the level of creatinine) in a sample from the subject. In some embodiments, the sample is a urine sample. In some embodiments, the sample is a plasma sample.

In some embodiments, the method decreases 5-ALA levels in the subject. In some embodiments, the method decreases 5-ALA levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases HMB levels in the subject. In some embodiments, the method decreases HMB levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases coproporphyrin III levels in the subject. In some embodiments, the method decreases coproporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases PBG levels in the subject. In some embodiments, the method decreases PBG levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases uroporphyrin III levels in the subject. In some embodiments, the method decreases uroporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases the proportion of protoporphyrin to coproporphyrin in the subject. In some embodiments, the method decreases the proportion of protoporphyrin to coproporphyrin in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases heptacarboxyl-porphyrin levels in the subject. In some embodiments, the method decreases heptacarboxyl-porphyrin levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases isocoproporphyrin levels in the subject. In some embodiments, the method decreases isocoproporphyrin levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases ZPPIX levels in the subject. In some embodiments, the method decreases ZPPIX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method decreases porphyrin or porphyrin precursor (e.g., ALA or PBG) levels in the subject to a normal level. In some embodiments, the normal level is a reference value for a porphyrin or porphyrin precursor (e.g., urine ALA levels <6.3 mmol/mol creatine and urine PBG levels <1.4 mmol/mol creatine) as described herein.

### Complications of Hepatic Porphyria

In certain aspects, the disclosure relates to methods of preventing, treating, or reducing the progression rate and/or severity of one or more complications of a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the one or more complications of a hepatic porphyria is selected from the group consisting of: acute photosensitivity, cutaneous photosensitivity, severe abdominal pain, neuropsychiatric symptoms, autonomic neuropathy, peripheral motor neuropathy, electrolyte disturbances, nausea, vomiting, constipation, diarrhea, difficulty urinating, ileus, paresthesia, insomnia, restlessness, agitation, anxiety, confusion, hallucinations, psychosis, convulsions, pain associated with neuropathy, muscle paralysis, tetraparesis, decreased breathing, respiratory arrest, hyponatremia, tachycardia, hypertension, increased heart rate, increased blood pressure, red urine, dark urine, hepatocellular carcinoma, hypertensive renal damage, chronic kidney disease, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, and terminal phase liver disease. In some embodiments, the one or more complications are improved indirectly. In some embodiments, the disclosure contemplates methods of preventing one or more complications of a hepatic porphyria comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the disclosure contemplates methods of reducing the progression rate of one or more complications of a hepatic porphyria comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the disclosure contemplates methods of reducing the severity of one or more complications of a hepatic porphyria comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt.

Methods of treatment provided herein may serve to ameliorate one or more symptoms associated with a hepatic porphyria or to reduce the risk of developing conditions associated with porphyria (e.g., neuropathy (e.g., progressive neuropathy), hepatocellular cancer). Symptoms associated with a hepatic porphyria may include abdominal pain or cramping, headaches, effects caused by nervous system abnormalities, and light sensitivity, causing rashes, blistering, and scarring of the skin (photodermatitis). In certain embodiments, the hepatic porphyria is AIP. Symptoms of AIP include gastrointestinal symptoms (e.g., severe and poorly localized abdominal pain, nausea/vomiting, constipation, diarrhea, ileus), urinary symptoms (dysuria, urinary retention/incontinence, or dark urine), neurologic symptoms (e.g., sensory neuropathy, motor neuropathy (e.g., affecting the cranial nerves and/or leading to weakness in the arms or legs), seizures, neuropathic pain, progressive neuropathy, headaches, neuropsychiatric symptoms (e.g., mental confusion, anxiety, agitation, hallucination, hysteria, delirium, apathy, depression, phobias, psychosis, insomnia, somnolence, coma), autonomic nervous system involvement (resulting e.g., in cardiovascular symptoms such as tachycardia, hypertension, and/or arrhythmias, as well as other symptoms, such as, e.g., increased circulating catecholamine levels, sweating, restlessness, and/or tremor), dehydration, and electrolyte abnormalities.

In some embodiments, a GlyT1 inhibitor is administered together with (e.g., before, after, or concurrent with) another treatment that may serve to alleviate one or more of the above symptoms. For example, abdominal pain may be treated, e.g., with narcotic analgesics, seizures may be treated, e.g., with anti-seizure medications, nausea/vomiting may be treated, e.g., with phenothiazines, and tachycardia/hypertension may be treated, e.g., with beta blockers.

Porphyrin photosensitization in certain hepatic porphyrias (e.g., VP, HCP, PCT, and HEP) may produce two distinct clinical syndromes: (1) acute photosensitivity on exposure to sunlight with erythema and edema and (2) a syndrome wherein subepidermal bullae occur in sun-exposed areas of the skin. In certain aspects, the disclosure relates to methods of preventing, treating, or reducing the progression rate and/or severity of a hepatic porphyria in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt, wherein the method increases pain free light exposure in the subject. In some embodiments, the method increases pain free light exposure in the subject by at least 10%, 20%, 30%, 40%, or 50% more as compared to pain free light exposure prior to administration of the GlyT1 inhibitor. In some embodiments, the method decreases light sensitivity in the subject. In some embodiments, the method decreases light sensitivity in the subject by at least 10%, 20%, 30%, 40%, or 50% more as compared to light sensitivity prior to administration of the GlyT1 inhibitor. In some embodiments, the subject has a history of phototoxic reactions from a hepatic porphyria. In some embodiments, the subject is an adult, child, infant, or pregnant woman.

### EC50 and Administration

In certain embodiments of the methods and uses as disclosed herein, the glycine transporter inhibitor, such as a GlyT1 inhibitor (e.g., a GlyT1 inhibitor as disclosed herein), or a pharmaceutically acceptable salt thereof, or a prodrug of the glycine transporter inhibitor, such as a GlyT1 inhibitor (e.g., a GlyT1 inhibitor as disclosed herein), or its pharmaceutically acceptable salt, demonstrates inhibition of a porphyrin precursor (e.g., 5-ALA or PBG) with an EC50 of less than 500 nM, less than 400 nM, less than 300 nM, less than 200 nM, or less than 100 nM. In certain embodiments of the present application, the glycine transporter inhibitor, such as a GlyT1 inhibitor (e.g., a GlyT1 inhibitor as disclosed herein), or a pharmaceutically acceptable salt thereof, or a prodrug of the glycine transporter inhibitor, such as a GlyT1 inhibitor (e.g., a GlyT1 inhibitor as disclosed herein), or its pharmaceutically acceptable salt, demonstrates inhibition of a porphyrin precursor (e.g., 5-ALA or PBG) with an EC50 of less than 100 nM. In certain embodiments of the present application, the glycine transporter inhibitor, such as a GlyT1 inhibitor (e.g., a GlyT1 inhibitor as disclosed herein), or a pharmaceutically acceptable salt thereof, or a prodrug of the glycine transporter inhibitor, such as a GlyT1 inhibitor (e.g., a GlyT1 inhibitor as disclosed herein), or its pharmaceutically acceptable salt, demonstrates inhibition of a porphyrin precursor (e.g., 5-ALA or PBG) with an EC50 of less than 50 nM. In certain embodiments, the EC50 is measured in a flow cytometry assay. In certain embodiments, the EC50 is measured in a LC-MS/MS assay. In certain embodiments of the foregoing, the GlyT1 inhibitor is bitopertin, or a pharmaceutically acceptable salt thereof, or a prodrug of bitopertin or its pharmaceutically acceptable salt.

In certain embodiments, the GlyT1 inhibitor is administered to prevent or reduce the severity or frequency of recurring attacks, e.g., cyclical attacks associated with a precipitating factor. In some embodiments, the precipitating factor is the menstrual cycle. In some embodiments, the GlyT1 inhibitor is administered repeatedly, e.g., at regular intervals to prevent or reduce the severity or frequency of recurring attacks, e.g., cyclical attacks associated with a precipitating factor, e.g., the menstrual cycle, e.g., a particular phase of the menstrual cycle, e.g., the luteal phase. In some embodiments, the GlyT1 inhibitor is administered during a particular phase of the menstrual cycle or based on hormone levels of the patient being treated (e.g., based on hormone levels that are associated with a particular phase of the menstrual cycle). In some embodiments, the GlyT1 inhibitor is administered on one or more particular days of the menstrual cycle, e.g., on day 1, 2, 3, 4, 5, 6, 7, 8, 9, 10, 11, 12, 13, 14, 15, 16, 17, 18, 19, 20, 21, 22, 23, 24, 25, 26, 27, or on day 28 (or later day for subjects who have a longer menstrual cycle). In some embodiments, the GlyT1 inhibitor is administered during the luteal phase, e.g., on one or more days between days 14-28 of the menstrual cycle (or later, in subjects who have a menstrual cycle longer than 28 days). In some embodiments, ovulation of the subject is assessed (e.g., using a blood or urine test that detects a hormone associated with ovulation, e.g., LH) and the GlyT1 inhibitor is administered at a predetermined interval after ovulation. In some embodiments, the GlyT1 inhibitor is administered immediately after ovulation. In some embodiments, the GlyT1 inhibitor is administered 1, 2, 3, 4, 5, 6, 7, 8, 9, 10, 11, 12, 13, 14, 15, 16, 17, or 18 days after ovulation. Any of these schedules may optionally be repeated for one or more iterations. The number of iterations may depend on the achievement of a desired effect, e.g., the achievement of a therapeutic or prophylactic effect, e.g., reduce or prevent one or more symptoms associated with a hepatic porphyria, to reduce the frequency of attacks associated with hepatic porphyria.

In some embodiments, an initial dose of GlyT1 inhibitor is administered and the level of ALA or PBG is tested, e.g., 1-48 hours, e.g., 2, 4, 8, 12, or 24 hours following administration of the initial dose. In some embodiments, if the level of ALA and/or PBG has decreased (e.g., to achieve a predetermined reduction, e.g., a normalization), and/or if the symptoms associated with a hepatic porphyria (e.g., pain) have improved (e.g., such that the patient is asymptomatic), no further dose is administered, whereas if the level of ALA and/or PBG has not decreased (e.g., has not achieved a predetermined reduction, e.g., has not normalized), a further dose of GlyT1 inhibitor is administered. In some embodiments, the further dose is administered 12, 24, 36, 48, 60, or 72 hours after the initial dose. In some embodiments, if the initial dose is not effective to decrease the level of ALA and/or PBG, the further dose is modified, e.g., increased to achieve a desired decrease (e.g., a predetermined reduction, e.g., a normalization) in ALA or PBG levels.

In some embodiments, the predetermined reduction is a decrease of at least 10%, 20%, 30%, 40%, or 50%. In some embodiments, the predetermined reduction is a reduction that is effective to prevent or ameliorate symptoms, e.g., pain, prodromal symptoms, or recurring attacks.

In some embodiments, the predetermined reduction is a reduction of at least 1, 2, 3, or more standard deviations, wherein the standard deviation is determined based on the values from a reference sample, e.g., a reference sample as described herein.

In some embodiments, the predetermined reduction is a reduction that brings the level of the porphyrin or porphyrin precursor to a level that is less than, or to a level that is less than or equal to, a reference value (e.g., a reference value as described herein).

As used herein, a “normalization” in ALA or PBG levels (or a “normal” or “normalized” level) refers to a level (e.g., a urine and/or plasma level) of either ALA, or PBG, or both, that is within the expected range for a healthy individual, an individual who is asymptomatic (e.g., an individual who does not experience pain and/or suffer from neuropathy), or an individual who does not have a mutation associated with a porphyria. For example, in some embodiments, a normalized level is within two standard deviations of the normal mean. In some embodiments, a normalized level is within normal reference limits, e.g., within the 95% confidence interval for an appropriate control sample, e.g., a sample of healthy individuals or individuals who do not carry a gene mutation associated with a porphyria. In some embodiments, the ALA and/or PBG level of the subject (e.g., the urine and/or plasma ALA and/or PBG level) is monitored at intervals, a further dose of the GlyT1 inhibitor agent is administered when the level increases above the reference value.

Administration of the GlyT1 inhibitor may reduce porphyrin or porphyrin precursor levels, e.g., in a cell, tissue, blood, urine or other compartment of the patient by at least 10%, at least 15%, at least 20%, at least 25%, at least 30%, at least 40%, at least 50%, at least 60%, at least 70%, at least 80% or at least 90% or more. Administration of the GlyT1 inhibitor may also decrease porphyrin or porphyrin precursor levels during an acute attack of AIP.

### Combination Therapy

Optionally, methods disclosed herein for preventing, treating, or reducing the progression rate and/or severity of one or more complications of a hepatic porphyria in a subject, may further comprise administering to the patient one or more supportive therapies or additional active agents for treating a hepatic porphyria. For example, the patient also may be administered one or more supportive therapies or active agents selected from the group consisting of: avoiding sunlight, topical sunscreens, skin protection, UVB phototherapy, Afamelanotide (Scenesse®), bortezomib, heme infusions, sufficient caloric support, Givosiran, RNAi mediated silencing of various enzymes (e.g., ALA synthase), avoiding precipitating factors, 4-aminoquinolines, chloroquine, hydroxychloroquine, phlebotomy, intravenous magnesium, LH-RH agonists, enzyme replacement therapy (e.g., recombinant human PBGD), gene therapy (e.g., transfer of PBGD gene in liver cells by viral vectors), hemodialysis, pharmacologic chaperone treatment, proteasome inhibitors, chemical chaperones, cholestyramine, activated charcoal, iron supplementation, liver transplantation, bone marrow transplantation, splenectomy, and blood transfusion.

In some embodiments, the subject is administered a combination treatment, e.g., a GlyT1 inhibitor as described herein, and one or more additional treatments known to be effective against hepatic porphyria (e.g., glucose and/or a heme product such as hemin, as described herein) or its associated symptoms.

In one embodiment, a GlyT1 inhibitor as described herein is administered in combination with glucose or dextrose. For example, 10-20% dextrose in normal saline may be provided intravenously. Typically, when glucose is administered, at least 300 g of 10% glucose is administered intravenously daily. The GlyT1 inhibitor may also be administered intravenously, as part of the same infusion that is used to administer the glucose or dextrose, or as a separate infusion that is administered before, concurrently, or after the administration of the glucose or dextrose. In some embodiments, the GlyT1 inhibitor is administered via a different route of administration (e.g., subcutaneously). In yet another embodiment, the GlyT1 inhibitor is administered in combination with total parenteral nutrition. The GlyT1 inhibitor may be administered before, concurrent with, or after the administration of total parenteral nutrition.

In certain embodiments, a GlyT1 inhibitor is administered in combination with one or more additional treatments, e.g., another treatment known to be effective in treating porphyria or symptoms of porphyria. In one embodiment, the GlyT1 inhibitor is administered in combination with a heme product (e.g., hemin, heme arginate, or heme albumin). In a further embodiment, the GlyT1 inhibitor is administered in combination with a heme product and glucose, a heme product and dextrose, or a heme product and total parenteral nutrition. The additional treatment(s) may be administered before, after, or concurrent with the administration of GlyT1 inhibitor. The GlyT1 inhibitor and an additional therapeutic agent can be administered in combination in the same composition, e.g., intravenously, or the additional therapeutic agent can be administered as part of a separate composition or by another method described herein. In some embodiments, the subject has previously been treated with a heme product (e.g., hemin, heme arginate, or heme albumin), as described herein.

In some embodiments, administration of the GlyT1 inhibitor, or administration of the GlyT1 inhibitor in combination one or more additional treatments (e.g., glucose, dextrose or the like), decreases the frequency of acute attacks (e.g., by preventing acute attacks so that they no longer occur, or by reducing the number of attacks that occur in a certain time period, e.g., fewer attacks occur per year). In some such embodiments, the GlyT1 inhibitor is administered according to a regular dosing regimen, e.g., b.i.d., daily, weekly, biweekly, or monthly.

### EXEMPLIFICATION

The invention now being generally described, it will be more readily understood by reference to the following examples, which are included merely for purposes of illustration of certain embodiments of the present invention, and are not intended to limit the invention.

### Example 1: Synthesis of Compounds

The compounds disclosed herein can be made in accordance with well known procedures and by processes known and disclosed in the art. For example, compounds of Formula I, such as bitopertin, can be prepared in accordance with the synthetic protocols provided in U.S. Pat. Nos. 7,319,099, 9,877,963, and 7,812,161, the contents of which are hereby incorporated by reference in their entirety. In addition, compounds of Formula II, such as PF-3463275, can be prepared in accordance with the synthetic protocols provided in U.S. Pat. No. 8,124,639, the contents of which are hereby incorporated by reference in its entirety.

### Example 2: Expression of GlyT1 in Liver Cells

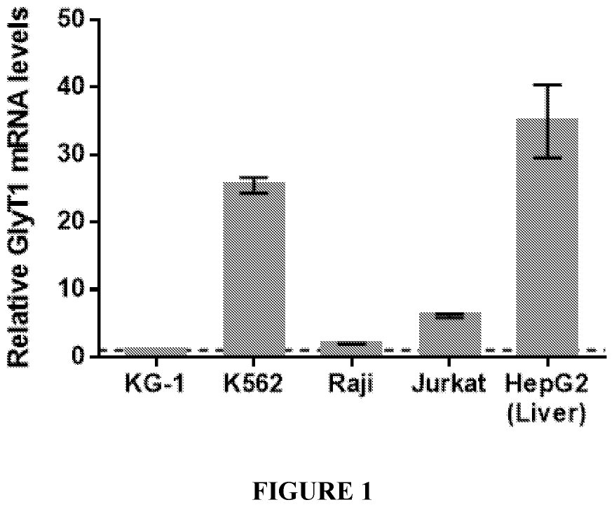

The liver is responsible for 15-20% of the heme synthesized in the human body, and as a result, enzymes of the heme synthesis pathway are generally expressed at high levels in the liver. Assessments of GlyT1 expression in the normal liver have tended to show no GlyT1 expression, and accordingly, it is generally thought that liver cells obtain glycine from internal metabolic sources and do not require the exogenous glycine source that GlyT1 expression would provide. Nonetheless, applicants assessed expression of GlyT1 in a variety of cell lines and surprisingly found substantial GlyT1 expression in a liver-derived cell line. See FIG. 1. The levels were higher than those observed in an erythropoietic cell line, K562. Id., GlyT1 is known to be an important source of glycine to support the induction of heme synthesis in conjunction with erythroid maturation. These data indicate that GlyT1 may be expressed transiently in liver cells, potentially in support of elevated heme synthesis demands, such as those that are characteristic of the hepatic porphyrias. Accordingly, administration of GlyT1 inhibitors has the potential to treat hepatic porphyrias.

Cells were cultured in the conditions described in Table 2 below and GlyT1 mRNA levels determined by standard methodology.

TABLE 2
Cell Lines and Cell Culture Conditions

| Cell | | | | |
|---|---|---|---|---|
| lines | Disease | Culture condition | Organism | Cell type |
| HepG2 | Hepatocellular | Eagle's Minimum | Homo | N.A. |
| | carcinoma | Essential Medium + | sapiens, | |
| | | 10% FBS + 1% PS | human | |
| KG-1 | Acute | Iscove's Modified | Homo | Macro- |
| | myelogenous | Dulbecco's Medium + | sapiens, | phage |
| | leukemia | 20% FBS + 1% PS | human | |
| K562 | Chronic | Iscove's Modified | Homo | N.A. |
| | myelogenous | Dulbecco's Medium + | sapiens, | |
| | leukemia | 10% FBS + 1% PS | human | |
| | (CML) | | | |
| Jurkat | Acute T cell | RPMI1640 Medium + | Homo | T |
| | leukemia | 10% FBS + 1% PS | sapiens, | lympho- |
| | | | human | cyte |
| Raji | Burkitt's | RPMI1640 Medium + | Homo | B |
| | lymphoma | 10% FBS + 1% PS | sapiens, | lympho- |
| | | | human | cyte |

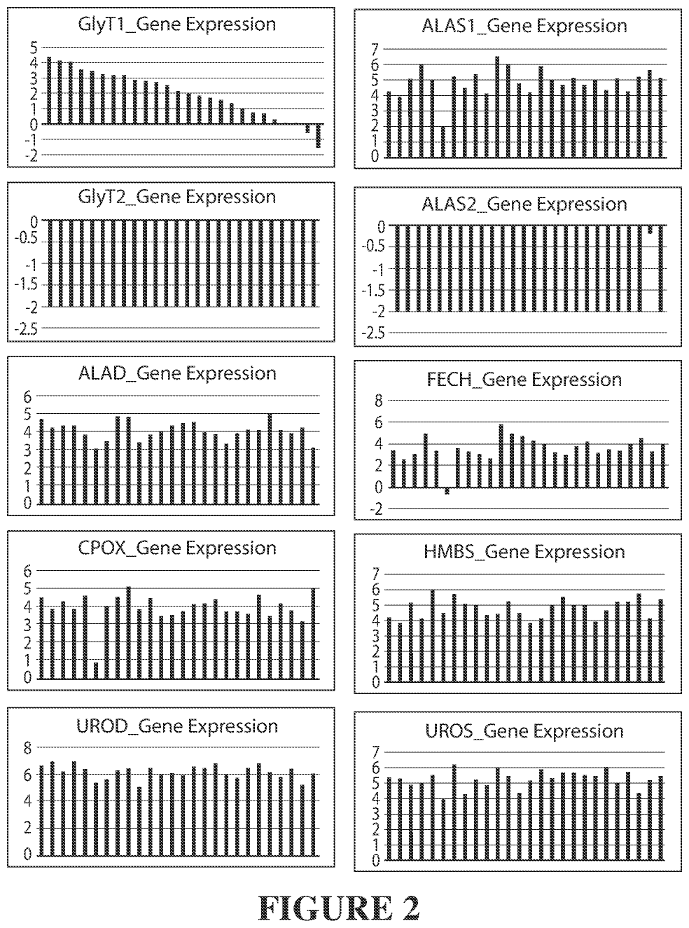

Applicants further assessed GlyT1 expression as reported in cancer cell databases and determined that GlyT1 is expressed in many of the 26 liver cancer cell lines analyzed. See FIG. 2. This data supports the initial conclusion that GlyT1 expression may be induced under certain conditions in normal liver cells. As positive controls, we note that other components of the heme synthesis pathway are expressed with near uniformity in liver cancer cell lines. Id. A second glycine transporter, GlyT2, is not expressed in liver cancer cell lines, indicating that GlyT2 is unlikely to play a role in heme synthesis in the liver. In general, GlyT2 is thought to be restricted to neural tissues.

### Example 3: Phenobarbital Induces Overexpression of ALAS1 and GlyT1 in HepG2 Cells

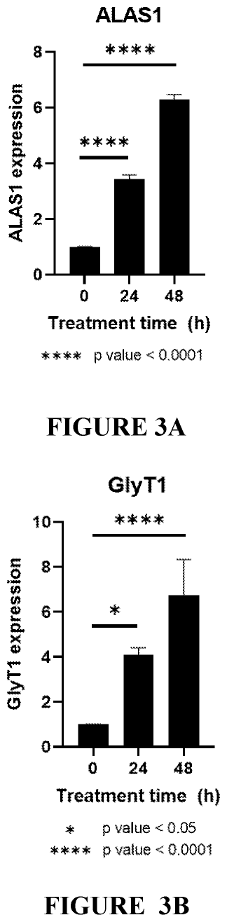

Phenobarbital is used to activate the heme synthesis pathway and induce AIP symptoms in the AIP murine model. It is known that phenobarbital stimulates the expression of ALAS1. The hepatic originated cell line HepG2 were treated with phenobarbital for 24 hours and 48 hours. The expression of ALAS1 and GlyT1 was examined by qPCR. The mRNA of ALAS1 (FIG. 3A) and GlyT1 (FIG. 3B) increased by 3- and 6-fold 24 h and 48 h after phenobarbital treatment. The result suggests GlyT1 overexpression induced by phenobarbital treatment may increase the intracellular glycine levels and subsequent heme pathway intermediates, therefore, contribute to the phenobarbital induced AIP symptoms.

### Example 4: Effect of GlyT1 Inhibitors on Glycine Uptake in Hepatic Cell Lines

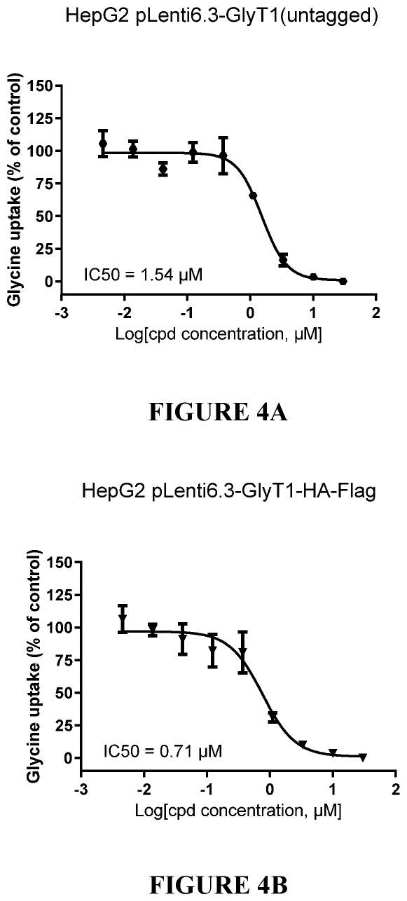

The hepatic originated cell line HepG2 cell was engineered via lentiviral infection to overexpress untagged GlyT1 (FIG. 4A) or HA-Flag-tagged GlyT1 (FIG. 4B). Overexpression of GlyT1 was used to mimic the phenobarbital induced AIP symptoms observed in HepG2 as described in Example 3. The HA-Flag tagged construct was used to confirm the overexpression of the GlyT1 protein by Western blot. Overexpression of the untagged construct was confirmed by qPCR. The cells were cultured in the presence of the GlyT1 inhibitor bitopertin at room temperature for 60 min and then incubated with 20 nM ³H-labeled glycine in the presence of 25 μM unlabeled glycine for 60 min. The uptake of glycine was measured by detection of radioisotope levels. In the presence of bitopertin, glycine uptake in the engineered HepG2 cells was significantly reduced, with the IC50 ranged from 0.71 to 1.54 μM (FIG. 4). The result suggests GlyT1 inhibition reduces the uptake of glycine in hepatic cells, which may reduce the intermediates in the heme synthesis pathway.

### Example 5: Effect of GlyT1 Inhibitors on Accumulation of Aminolaevulinic Acid (5-ALA) and Porphobilinogen (PBG) in AIP Cellular Model

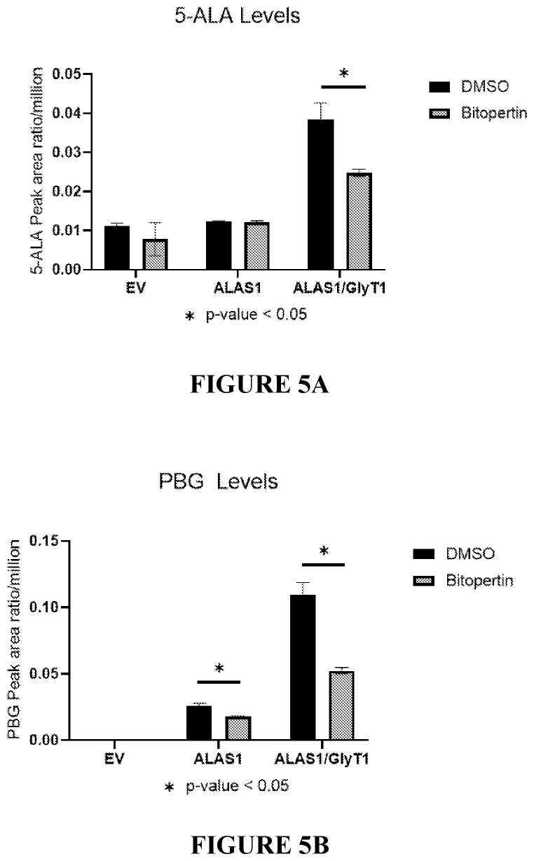

The hepatic origin cell line HepG2 was engineered to express shRNA against HMBS, a gene in the heme biosynthesis pathway whose loss-of-function mutations cause hepatic porphyria. The shRNA reduced HMBS mRNA by 50% and HMBS protein by 70%. Hepatic porphyria is a dormant disease activated by increased expression of ALAS1. To establish the cellular model that resembles the pathologic status of hepatic porphyria, the HepG2 cells with HMBS knockdown were also transduced by lentivirus to overexpress ALAS1 and GlyT1. The genetically modified HepG2 cells were confirmed to model the pathologic status of hepatic porphyria by demonstrating the increased production of 5-ALA (FIG. 5A) and PBG (FIG. 5B), toxic heme pathway metabolites associated with the hepatic porphyria disease. Treatment of the modified HepG2 cells with the GlyT1 inhibitor bitopertin was shown to significantly decrease production of the toxic metabolites.

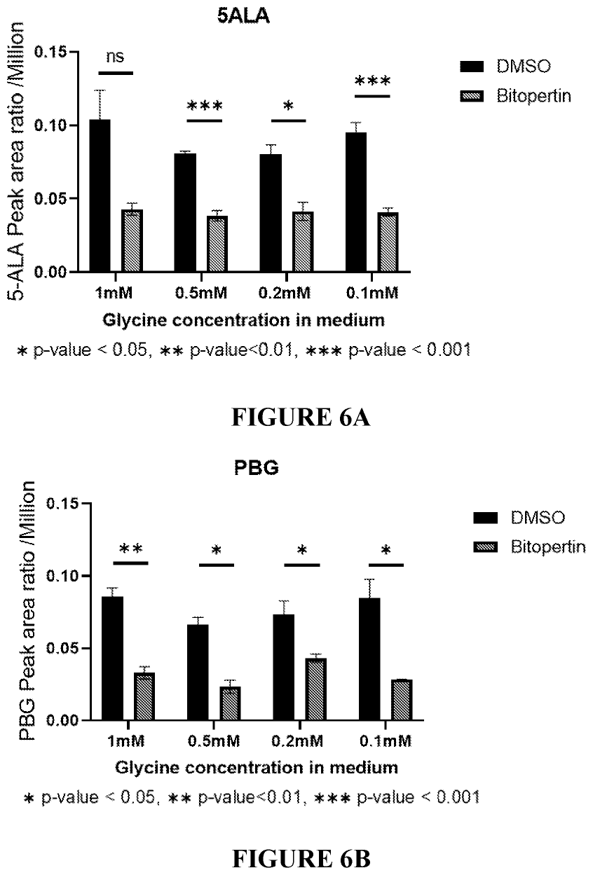

The normal plasma glycine concentration in human adults ranges from 0.12 to 0.55 mM. To confirm GlyT1 inhibitors such as bitopertin can decrease production of toxic metabolites at physiological condition, the modified HepG2 cells were treated with bitopertin in the presence of different concentrations of glycine (0.1-1 mM). Bitopertin treatment showed consistent reduction of toxic metabolites 5-ALA (FIG. 6A) and PBG (FIG. 6B), regardless of glycine concentration in the medium.

Together, these data demonstrate that inhibiting glycine uptake suppresses the production of heme and its metabolic intermediates. Therefore, GlyT1 inhibitors such as bitopertin may have utility in controlling the production of toxic metabolites in patients with hepatic porphyria.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method of treating a hepatic porphyria in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt.

2. A method of preventing, treating, or reducing the progression rate and/or severity of a hepatic porphyria in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt.

3. A method of preventing, treating, or reducing the progression rate and/or severity of one or more complications of a hepatic porphyria in a subject, the method comprising administering to the subject a pharmaceutical composition comprising one or more GlyT1 inhibitor, or a pharmaceutically acceptable salt thereof, or a prodrug of the one or more GlyT1 inhibitor or its pharmaceutically acceptable salt.

4. The method of claim 3, wherein the one or more complications of hepatic porphyria is selected from the group consisting of: acute photosensitivity, cutaneous photosensitivity, severe abdominal pain, neuropsychiatric symptoms, autonomic neuropathy, peripheral motor neuropathy, electrolyte disturbances, nausea, vomiting, constipation, diarrhea, difficulty urinating, ileus, paresthesia, insomnia, restlessness, agitation, anxiety, confusion, hallucinations, psychosis, convulsions, pain associated with neuropathy, muscle paralysis, tetraparesis, decreased breathing, respiratory arrest, hyponatremia, tachycardia, hypertension, increased heart rate, increased blood pressure, red urine, dark urine, hepatocellular carcinoma, hypertensive renal damage, chronic kidney disease, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, and terminal phase liver disease.

5. The method of any one of claims 1-4, wherein the hepatic porphyria is an acute hepatic porphyria.

6. The method of claim 5, wherein the acute hepatic porphyria is acute intermittent porphyria (AIP).

7. The method of claim 5, wherein the acute hepatic porphyria is ALA dehydratase porphyria (ADP).

8. The method of claim 5, wherein the acute hepatic porphyria is variegate porphyria (VP).

9. The method of claim 5, wherein the acute hepatic porphyria is hereditary coproporphyria (HCP).

10. The method of claim 5, wherein the acute hepatic porphyria is harderoporphyria.

11. The method of any one of claims 1-4, wherein the hepatic porphyria is non-acute hepatic porphyria.

12. The method of claim 11, wherein the non-acute hepatic porphyria is familial and sporadic porphyria cutanea tarda (PCT).

13. The method of claim 11, wherein the non-acute hepatic porphyria is hepatoerythropoietic porphyria (HEP).

14. The method of claim 4, wherein the acute photosensitivity is due to sun exposure.

15. The method of any one of claims 4, 13 or 14, wherein the method increases pain free light exposure in the subject.

16. The method of any one of claims 1-15, wherein the method decreases light sensitivity in the subject.

17. A method of inhibiting 5-aminolevulinic acid (5-ALA) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has a hepatic porphyria.

18. A method of inhibiting coproporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt.

19. A method of inhibiting zinc-protoporphyrin IX (ZPPIX) synthesis in a subject, comprising administering to a subject a GlyT1 inhibitor, or a pharmaceutically acceptable salt thereof, or a prodrug of the GlyT1 inhibitor or its pharmaceutically acceptable salt, wherein the subject has ALA dehydratase porphyria (ADP).

20. A method of inhibiting porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt.

21. A method of inhibiting 5-aminolevulinic acid (5-ALA) and porphobilinogen (PBG) synthesis in vivo, comprising administering to a subject a GlyT1 inhibitor, or a pharmaceutically acceptable salt thereof, or a prodrug of the GlyT1 inhibitor or its pharmaceutically acceptable salt.

22. A method of inhibiting hydroxymethylbilane (HMB) synthesis in vivo, comprising administering to a subject a GlyT1 inhibitor, or a pharmaceutically acceptable salt thereof, or a prodrug of the GlyT1 inhibitor or its pharmaceutically acceptable salt.

23. A method of inhibiting uroporphyrin III synthesis in vivo, comprising administering to a subject a GlyT1 inhibitor, or a pharmaceutically acceptable salt thereof, or a prodrug of the GlyT1 inhibitor or its pharmaceutically acceptable salt.

24. A method of inhibiting heptacarboxyl-porphyrin synthesis in vivo, comprising administering to a subject a GlyT1 inhibitor, or a pharmaceutically acceptable salt thereof, or a prodrug of the GlyT1 inhibitor or its pharmaceutically acceptable salt.

25. A method of inhibiting isocoproporphyrin synthesis in vivo, comprising administering to a subject a GlyT1 inhibitor, or a pharmaceutically acceptable salt thereof, or a prodrug of the GlyT1 inhibitor or its pharmaceutically acceptable salt.

26. A method of inhibiting synthesis of a porphyrin or porphyrin precursor in vivo, comprising administering to a subject a GlyT1 inhibitor, or a pharmaceutically acceptable salt thereof, or a prodrug of the GlyT1 inhibitor or its pharmaceutically acceptable salt, wherein the porphyrin or porphyrin precursor is selected from the group consisting of:
a. 5-ALA
b. PBG
c. Hydroxymethylbilane
d. ZPPIX
e. Uroporphyrinogen I
f. Uroporphyrinogen III
g. Heptacarboxyporphyrinogen I
h. Heptacarboxyporphyrinogen III
i. Hexacarboxyporphyrinogen I
j. Hexacarboxyporphyrinogen III
k. Pentacarboxyporphyrinogen I
l. Pentacarboxyporphyrinogen III
m. Coproporphyrinogen I
n. Coproporphyrinogen III
o. Isocoproporphyrin
p. Porphobilinogen; and
q. Protoporphyrinogen IX.

27. The method of any one of claims 1-26, wherein the accumulation of one or more heme intermediates is inhibited, and wherein the one or more heme intermediates are selected from the group consisting of 5-ALA, coproporphyrin III, zinc-protoporphyrin IX (ZPPIX), porphobilinogen, uroporphyrin III, heptacarboxyl-porphyrin, and isocoproporphyrin.

28. The method of any one of claims 1-26, wherein the accumulation of one or more heme intermediates is inhibited, and wherein the one or more heme intermediates are selected from the group consisting of:
a. 5-ALA
b. PBG
c. Hydroxymethylbilane
d. ZPPIX
e. Uroporphyrinogen I
f. Uroporphyrinogen III
g. Heptacarboxyporphyrinogen I
h. Heptacarboxyporphyrinogen III
i. Hexacarboxyporphyrinogen I
j. Hexacarboxyporphyrinogen III
k. Pentacarboxyporphyrinogen I
l. Pentacarboxyporphyrinogen III
m. Coproporphyrinogen I
n. Coproporphyrinogen III
o. Isocoproporphyrin
p. Porphobilinogen; and
q. Protoporphyrinogen IX.

29. The method of claim 27 or claim 28, wherein the accumulation of the one or more heme intermediates is inhibited in a dose dependent manner.

30. The method of any preceding claim, wherein the GlyT1 inhibitor demonstrates an EC50 of less than 500 nM.

31. The method of any preceding claim, wherein the GlyT1 inhibitor demonstrates an EC50 of less than 100 nM.

32. The method of any one of claims 1-31, wherein the subject has or is at risk for developing a hepatic porphyria and suffers from pain (e.g., neuropathic pain, e.g., chronic neuropathic pain) or neuropathy (e.g., progressive neuropathy).

33. The method of any one of claims 1-32, wherein the subject has an elevated level of ALA and/or PBG and suffers from chronic pain.

34. The method of any one of claims 1-33, wherein the subject has 5-ALA levels that are at least 10%, 20%, 30%, 40%, or 50% more than 5-ALA levels in a healthy subject prior to administration of the GlyT1 inhibitor.

35. The method of any one of claims 1-34, wherein the subject has HMB levels that are at least 10%, 20%, 30%, 40%, or 50% more than HMB levels in a healthy subject prior to administration of the GlyT1 inhibitor.

36. The method of any one of claims 1-35, wherein the subject has coproporphyrin III levels that are at least 10%, 20%, 30%, 40%, or 50% more than coproporphyrin III levels in a healthy subject prior to administration of the GlyT1 inhibitor.

37. The method of any one of claims 1-36, wherein the subject has ZPPIX levels that are at least 10%, 20%, 30%, 40%, or 50% more than ZPPIX levels in a healthy subject prior to administration of the GlyT1 inhibitor.

38. The method of any one of claims 1-37, wherein the subject has porphobilinogen levels that are at least 10%, 20%, 30%, 40%, or 50% more than porphobilinogen levels in a healthy subject prior to administration of the GlyT1 inhibitor.

39. The method of any one of claims 1-38, wherein the subject has uroporphyrin III levels that are at least 10%, 20%, 30%, 40%, or 50% more than uroporphyrin III levels in a healthy subject prior to administration of the GlyT1 inhibitor.

40. The method of any one of claims 1-39, wherein the subject has heptacarboxyl-porphyrin levels that are at least 10%, 20%, 30%, 40%, or 50% more than heptacarboxyl-porphyrin levels in a healthy subject prior to administration of the GlyT1 inhibitor.

41. The method of any one of claims 1-40, wherein the subject has isocoproporphyrin levels that are at least 10%, 20%, 30%, 40%, or 50% more than isocoproporphyrin levels in a healthy subject prior to administration of the GlyT1 inhibitor.

42. The method of any one of claims 1-41, wherein the subject's heme levels are substantially maintained during treatment.

43. The method of any one of claims 1-41, wherein the treatment decreases subject's heme levels decrease no more than 10% (e.g., 10%, 15%, 20%, 25%, and 30%).

44. The method of any one of claims 1-41, wherein the dosage of the pharmaceutical composition does not cause a substantial reduction in heme levels.

45. The method of any one of claims 1-44, wherein the subject has increased 5-ALA levels.

46. The method of any one of claims 1-45, wherein the subject has increased 5-ALA levels in the urine.

47. The method of any one of claims 1-46, wherein the subject has increased 5-ALA levels in the plasma.

48. The method of any one of claims 1-47, wherein the subject has increased HMB levels.

49. The method of any one of claims 1-48, wherein the subject has increased coproporphyrin III levels.

50. The method of any one of claims 1-49, wherein the subject has increased coproporphyrin III levels in the urine.

51. The method of any one of claims 1-50, wherein the subject has increased coproporphyrin III levels in the stool.

52. The method of any one of claims 1-51, wherein the subject has increased porphobilinogen (PBG) levels.

53. The method of any one of claims 1-52, wherein the subject has increased porphobilinogen (PBG) levels in the urine.

54. The method of any one of claims 1-53, wherein the subject has a plasma level or a urine level of 5-ALA or PBG that is greater than a reference value.

55. The method of claim 54, wherein the reference value is two standard deviations above the mean level in a sample of healthy individuals.

56. The method of any one of claims 1-55, wherein the subject has a plasma level or a urine level of 5-ALA or PBG that is greater than or equal to 2 times, 3 times, 4 times, or 5 times that of an upper reference limit.

57. The method of any one of claims 1-55, wherein the subject has a urine level of PBG that is greater than or equal to 4.8 mmol/mol creatinine.

58. The method of any one of claims 1-55, wherein the subject has a plasma PBG level of greater than or equal to 0.12 μmol/L.

59. The method of any one of claims 1-55, wherein the subject has a urine PBG level of greater than or equal to 1.2 mmol/mol creatinine.

60. The method of any one of claims 1-55, wherein the subject has a plasma 5-ALA level of greater than or equal to 0.12 μmol/L.

61. The method of any one of claims 1-55, wherein the subject has a urine 5-ALA level of greater than or equal to 3.1 mmol/mol creatinine.

62. The method of any one of claims 1-55, wherein the method decreases the elevated level of 5-ALA and/or PBG.

63. The method of any one of claims 1-62, wherein the subject has increased uroporphyrin III levels.

64. The method of any one of claims 1-63, wherein the subject has increased uroporphyrin III levels in the urine.

65. The method of any one of claims 1-64, wherein the subject has an increased proportion of protoporphyrin to coproporphyrin in the stool.

66. The method of any one of claims 1-65, wherein the subject has increased heptacarboxyl-porphyrin levels.

67. The method of any one of claims 1-66, wherein the subject has increased heptacarboxyl-porphyrin levels in the urine.

68. The method of any one of claims 1-67, wherein the subject has increased heptacarboxyl-porphyrin levels in the stool.

69. The method of any one of claims 1-68, wherein the subject has increased isocoproporphyrin levels.

70. The method of any one of claims 1-69, wherein the subject has increased isocoproporphyrin levels in the stool.

71. The method of any one of claims 1-70, wherein the subject has increased ZPPIX levels in erythrocytes.

72. The method of any one of claims 1-71, wherein the method decreases 5-ALA levels in the subject.

73. The method of any one of claims 1-72, wherein the method decreases 5-ALA levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

74. The method of any one of claims 1-73, wherein the method decreases HMB levels in the subject.

75. The method of any one of claims 1-74, wherein the method decreases HMB levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

76. The method of any one of claims 1-75, wherein the method decreases coproporphyrin III levels in the subject.

77. The method of any one of claims 1-76, wherein the method decreases coproporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

78. The method of any one of claims 1-77, wherein the method decreases PBG levels in the subject.

79. The method of any one of claims 1-78, wherein the method decreases PBG levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

80. The method of any one of claims 1-79, wherein the method is effective to decrease the level of 5-ALA and/or PBG.

81. The method of any one of claims 1-80, wherein the level of 5-ALA and/or PBG is decreased such that it falls below a reference value.

82. The method of claim 81, wherein the reference value is an upper reference limit.

83. The method of any one of claims 1-82, wherein the method decreases uroporphyrin III levels in the subject.

84. The method of any one of claims 1-83, wherein the method decreases uroporphyrin III levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

85. The method of any one of claims 1-84, wherein the method decreases the proportion of protoporphyrin to coproporphyrin in the subject.

86. The method of any one of claims 1-85, wherein the method decreases the proportion of protoporphyrin to coproporphyrin in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

87. The method of any one of claims 1-86, wherein the method decreases heptacarboxyl-porphyrin levels in the subject.

88. The method of any one of claims 1-87, wherein the method decreases heptacarboxyl-porphyrin levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

89. The method of any one of claims 1-88, wherein the method decreases isocoproporphyrin levels in the subject.

90. The method of any one of claims 1-89, wherein the method decreases isocoproporphyrin levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

91. The method of any one of claims 1-90, wherein the method decreases ZPPIX levels in the subject.

92. The method of any one of claims 1-91, wherein the method decreases ZPPIX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

93. The method of any one of claims 1-92, wherein the subject's plasma porphyrin fluoresces at a peak between 615 nm and 620 nm when illuminated with blue light (e.g., 400-420 nm light).

94. The method of any one of claims 1-92, wherein the subject's plasma porphyrin fluoresces at a peak between 624 nm and 627 nm when illuminated with blue light (e.g., 400-420 nm light).

95. The method of any one of claims 1-92, wherein the subject's skin porphyrin fluoresces at a peak between 615 nm and 620 nm when illuminated with blue light (e.g., 400-420 nm light).

96. The method of any one of claims 1-92, wherein the subject's skin porphyrin fluoresces at a peak between 624 nm and 627 nm when illuminated with blue light (e.g., 400-420 nm light).

97. The method of any one of claims 1-96, wherein the subject has a defect in an enzyme selected from the group consisting of:
a. ALA-dehydratase
b. PBG deaminase
c. Uroporphyrinogen III synthase
d. Uroporphyrinogen decarboxylase
e. Coproporphyrinogen oxidase; and
f. Protoporphyrinogen oxidase.

98. The method of any one of claims 1-97, wherein the subject has mutation in a gene selected from the group consisting of:
a. ALAD
b. HMBS
c. UROS
d. UROD
e. CPOX, and
f. PPOX.

99. The method of any one of claims 1-98, wherein the GlyT1 inhibitor is administered after an acute attack.

100. The method of any one of claims 1-98, wherein the GlyT1 inhibitor is administered during an acute attack.

101. The method of any one of claims 1-98, wherein the GlyT1 inhibitor is administered during a prodrome.

102. The method of claim 101, wherein the prodrome is characterized by pain (e.g., headache and/or abdominal pain), nausea, psychological symptoms (e.g., anxiety), restlessness and/or insomnia.

103. The method of any one of claims 1-98, wherein the GlyT1 inhibitor is administered prophylactically to prevent an acute attack of hepatic porphyria.

104. The method of any one of claims 1-98, wherein the GlyT1 inhibitor is administered during a particular phase of the menstrual cycle, e.g., during the luteal phase.

105. The method of any one of claims 1-98, wherein the GlyT1 inhibitor ameliorates or prevents cyclical attacks of hepatic porphyria.

106. The method of claim 105, wherein the cyclical attacks are associated with a precipitating factor.

107. The method of claim 106, wherein the precipitating factor is a particular phase of the menstrual cycle, e.g., the luteal phase.

108. The method of claim 106, wherein the precipitating factor is the premenstrual phase.

109. The method of claim 106, wherein the precipitating factor is exposure to a chemical.

110. The method of claim 106, wherein the precipitating factor is exposure to lead.

111. The method of claim 106, wherein the precipitating factor is selected from the group consisting of drugs, xenobiotics, steroid hormones, smoking, alcohol, decreased intake of calories or carbohydrates, fasting, metabolic stress, and psychological stress.

112. The method of any one of claims 1-111, wherein the method decreases pain or neuropathy.

113. The method of any one of claims 1-112, wherein the method prevents acute attacks of hepatic porphyria.

114. The method of any one of claims 1-113, wherein the method decreases or prevents nerve damage.

115. The method of any one of claims 1-114, wherein the GlyT1 inhibitor is administered prophylactically beginning at puberty.

116. The method of any one of claims 1-115, comprising further administering to the subject an additional active agent and/or supportive therapy.

117. The method of claim 116, wherein the additional active agent and/or supportive therapy is selected from the group consisting of: avoiding sunlight, topical sunscreens, skin protection, UVB phototherapy, Afamelanotide (Scenesse®), bortezomib, heme infusions, sufficient caloric support, Givosiran, RNAi mediated silencing of various enzymes (e.g., ALA synthase), avoiding precipitating factors, 4-aminoquinolines, chloroquine, hydroxychloroquine, phlebotomy, intravenous magnesium, LH-RH agonists, enzyme replacement therapy (e.g., recombinant human PBGD), gene therapy (e.g., transfer of PBGD gene in liver cells by viral vectors), hemodialysis, pharmacologic chaperone treatment, proteasome inhibitors, chemical chaperones, cholestyramine, activated charcoal, iron supplementation, liver transplantation, bone marrow transplantation, splenectomy, and blood transfusion.

118. The method of any one of claims 1-117, wherein the GlyT1 inhibitor is a compound having a formula of 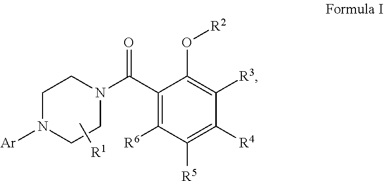
wherein:
Ar is unsubstituted or substituted aryl or 6-membered heteroaryl containing one, two or three nitrogen atoms, wherein the substituted aryl and the substituted heteroaryl groups are substituted by one or more substituents selected from the group consisting of hydroxy, halogen, NO₂, CN, (C₁-C₆)-alkyl, (C₁-C₆)-alkyl substituted by halogen, (C₁-C₆)-alkyl substituted by hydroxy, (CH₂)n-(C₁-C₆)-alkoxy, (C₁-C₆)-alkoxy substituted by halogen, NR⁷R⁸, C(O)R⁹, SO2R¹⁰, and —C(CH₃)=NOR⁷, or are substituted by a 5-membered aromatic heterocycle containing 1-4 heteroatoms selected from N and O, which is optionally substituted by (C₁-C₆)-alkyl; R¹ is hydrogen or (C₁-C₆)-alkyl;
R² is hydrogen, (C₁-C₆)-alkyl, (C₂-C₆)-alkenyl, (C₁-C₆)-alkyl substituted by halogen, (C₁-C₆)-alkyl substituted by hydroxy, (CH2)n-(C₃-C₇)-cycloalkyl optionally substituted by (C₁-C₆)-alkoxy or by halogen, CH(CH₃)—(C₃-C₇)-cycloalkyl, (CH₂)ₙ₊₁—C(O)—R⁹, (CH₂)ₙ₊₁—CN, bicyclo[2.2.1]heptyl, (CH₂)ₙ₊₁—O—(C₁-C₆)-alkyl, (CH₂)ₙ-heterocycloalkyl, (CH₂)ₙ-aryl or (CH₂)ₙ-5 or 6-membered heteroaryl containing one, two or three heteroatoms selected from the group consisting of oxygen, sulphur or nitrogen wherein aryl, heterocycloalkyl and heteroaryl are unsubstituted or substituted by one or more substituents selected from the group consisting of hydroxy, halogen, (C₁-C₆)-alkyl and (C₁-C₆)-alkoxy;
R³, R⁴ and R⁶ are each independently hydrogen, hydroxy, halogen, (C₁-C₆)-alkyl, (C₁-C₆)-alkoxy or O—(C₃-C₆)-cycloalkyl;
R⁵ is NO₂, CN, C(O)R⁹ or SO₂R¹⁰;
R⁷ and R⁸ are each independently hydrogen or (C₁-C₆)-alkyl;
R⁹ is hydrogen, (C₁-C₆)-alkyl, (C₁-C₆)-alkoxy or NR⁷R⁸;
R¹⁰ is (C₁-C₆)-alkyl optionally substituted by halogen, (CH₂)ₙ—(C₃-C₆)-cycloalkyl, (CH₂)ₙ—(C₃-C₆)-alkoxy, (CH₂)ₙ-heterocycloalkyl or NR⁷R⁸;
n is 0, 1, or 2;
or a pharmaceutically acceptable salt thereof, or a prodrug of the compound or its pharmaceutically acceptable salt.

119. The method of claim 118, wherein the GlyT1 inhibitor is a compound having a formula of 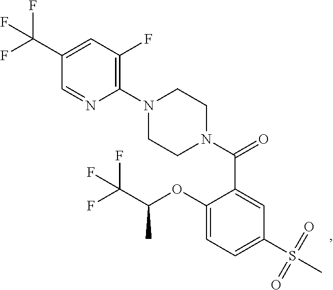 [SMILES: C[C@H](Oc1ccc(S(C)(=O)=O)cc1C(=O)N1CCN(c2ncc(C(F)(F)F)cc2F)CC1)C(F)(F)F]
bitopertin, or a pharmaceutically acceptable salt thereof, or a prodrug of the compound or its pharmaceutically acceptable salt.

120. The method of any one of claims 1-119, wherein the pharmaceutical composition further comprises a pharmaceutically acceptable carrier.

121. The method of any one of claims 1-120, wherein the subject is a subject in need thereof.

122. The method of any one of claims 1-121, wherein the GlyT1 inhibitor, or pharmaceutically acceptable salt thereof, or prodrug of the GlyT1 inhibitor or its pharmaceutically acceptable salt, is administered in a therapeutically effective amount.
